(19) United States
(12) Patent Application Publication
(10) Pub. No.: US 2020/0254081 A1
(43) Pub. Date: Aug. 13, 2020

(54) COMPOSITIONS AND METHODS FOR ELICITING AN IMMUNE RESPONSE AGAINST CLOSTRIDIUM DIFFICILE

(71) Applicant: Pfizer Inc., New York, NY (US)

(72) Inventors: Kathrin Ute Jansen, New York, NY (US); Annaliesa Sybil Anderson, Upper Saddle River, NJ (US); Robert G.K. Donald, South Orange, NJ (US); Michael James Flint, Decatur, GA (US); Nicholas Randolph Everard Kitchin, Sandwich (GB); Justin Keith Moran, West Nyack, NY (US); Louise Pedneault, New York, NY (US); Michael W. Pride, Staten Island, NY (US); Mark Edward Ruppen, Cresco, PA (US); Christopher Frederick Webber, Sandwich (GB)

(21) Appl. No.: 16/647,124

(22) PCT Filed: Sep. 14, 2018
(86) PCT No.: PCT/IB2018/057076

§ 371 Date: Mar. 13, 2020

Related U.S. Application Data
(60) Provisional application No. 62/720,617, filed on Aug. 21, 2018.
(60) Provisional application No. 62/577,661, filed on Oct. 26, 2017.
(60) Provisional application No. 62/576,603, filed on Oct. 24, 2017.
(60) Provisional application No. 62/565,096, filed on Sep. 28, 2017.

(51) Int. Cl. A61K 39/08; A61K 9/19

(52) U.S. Cl. CPC A61K 39/08; A61K 2039/545; A61K 9/19

(57) ABSTRACT

In one aspect, the invention relates to an immunogenic composition that includes a Clostridium difficile toxoid A and/or a C. difficile toxoid B, and methods of use thereof. In another aspect, the invention relates to a method for eliciting an immune response in a human against a C. difficile infection. The method includes administering to the human an effective dose of a composition, which includes a C. difficile toxoid, wherein the composition is administered at least two times, wherein the second administration is about 30 days after the first administration, and wherein the immune response against C. difficile toxin A and/or toxin B is sustained.

### CROSS REFERENCE TO RELATED APPLICATIONS

This application is a U.S. national phase application under 35 U.S.C. § 371 of International Application No. PCT/IB2018/057076, filed on Sep. 14, 2018, which claims priority to U.S. Provisional Application No. 62/577,661, filed on Sep. 28, 2017, U.S. Provisional Application No. 62/576,603, filed on Oct. 24, 2017, U.S. Provisional Application No. 62/577,661, filed on Oct. 26, 2017, and U.S. Provisional Application No. 62/720,617, filed on Aug. 21, 2018. Each of these patent applications is herein incorporated by reference in its entirety.

### FIELD

The present invention is directed to compositions and methods concerning Clostridium difficile toxoids and methods thereof.

### BACKGROUND

Clostridium difficile (C. difficile) is a Gram-positive anaerobic bacterium that is associated with gastrointestinal disease in humans. Colonization of C. difficile usually occurs in the colon if the natural gut flora is diminished by treatment with antibiotics. An infection can lead to antibiotic-associated diarrhea and sometimes pseudomembranous colitis through the secretion of the glucosylating toxins, toxin A and toxin B (308 and 270 kDa, respectively), which are the primary virulence factors of C. difficile.

In the last decade, the numbers and severity of C. difficile outbreaks in hospitals, nursing homes, and other long-term care facilities increased dramatically. Key factors in this escalation include emergence of hypervirulent pathogenic strains, increased use of antibiotics, improved detection methods, and increased exposure to airborne spores in health care facilities.

Metronidazole and vancomycin represent the currently accepted standard of care for the antibiotic treatment of C. difficile associated disease (CDAD). However, about 20% of patients receiving such treatment experience a recurrence of infection after a first episode of CDI, and up to about 50% of those patients suffer from additional recurrences. Treatment of recurrences represents a very significant challenge, and the majority of recurrences usually occur within one month of the preceding episode.

Humoral immune responses to C. difficile toxins play a significant role in preventing a more severe outcome or a recurrence of the disease in humans. Clinical studies suggest a correlation between high serum concentrations of anti-toxin A immunoglobulin G (as measured by enzyme linked immunosorbent assay) and protection from CDI or recurrence after primary CDI. Active immunization with inactivated toxins and passive immunization with anti-toxin antibodies have been demonstrated to protect animals from lethal challenge. In addition, hamsters treated with vancomycin alone have a higher mortality rate compared to those treated with vancomycin plus anti-toxin antibodies, indicating that vaccination may provide an advantage over antibiotics.

An approved highly effective noninvasive treatment for complicated C. difficile infection (CDI) does not exist. Accordingly, there is a need for immunogenic and/or therapeutic compositions and methods thereof directed to C. difficile.

### SUMMARY OF THE INVENTION

To meet these and other needs, the present invention relates to C. difficile toxoids and methods of use thereof. As used herein, the terms “toxoid,” “mutant toxin,” and “polypeptide” are synonymous and are used interchangeably unless otherwise stated. In one aspect, the invention relates to a C. difficile vaccine currently being evaluated for efficacy and safety in subjects who are at risk for CDI. The selection of an optimal vaccination dose and regimen were based on studies, taking into consideration immunogenicity, safety, and the potential for short- and long-term protection.

In one aspect, the invention relates to a method for eliciting an immune response in a human against a Clostridium difficile infection. The method includes administering to the human an effective dose of a composition, which includes a C. difficile toxoid, i.e., a polypeptide, wherein the composition is administered at least two times. In one embodiment, the second administration is at least 7 days after the first administration and the third administration is about 30 days after the first administration. In one embodiment, the third administration is about 180 days after the first administration. In one embodiment, the composition is administered at least three times. In one embodiment, the second administration is about 30 days after the first administration and the third administration is about 180 days after the first or second administration. In one embodiment, the third administration is at least 180 days after the first administration.

In one embodiment, the immune response elicited includes an anti-toxin A neutralizing monoclonal antibody. In one embodiment, the immune response elicited includes an anti-toxin B neutralizing monoclonal antibody. In one embodiment, the immune response elicited includes an anti-toxin A neutralizing monoclonal antibody and an anti-toxin B neutralizing monoclonal antibody, wherein the concentration of neutralizing monoclonal antibody is at least 10 μg/mL.

In one embodiment, the composition includes a C. difficile toxoid A and/or a C. difficile toxoid B, each having a purity of at least 90% or greater. In one embodiment, the composition includes a C. difficile toxoid A and/or a C. difficile toxoid B, in a ratio of about 3:1 to about 1:1.

The method according to claim 1 wherein the composition includes a C. difficile toxoid A and/or a C. difficile toxoid B, in a ratio of 1:1. In one embodiment, the composition includes an adjuvant. In one embodiment, the composition includes an aluminum adjuvant.

In one embodiment, the immune response against C. difficile toxin A and/or toxin B is sustained for at least about 60 days. In one embodiment, the immune response against C. difficile toxin A and/or toxin B is sustained for at least about 180 days after the first dose. In one embodiment, the immune response against C. difficile toxin A and/or toxin B is sustained for at least about 180 days after the second dose. In one embodiment, the immune response against C. difficile toxin A and/or toxin B is sustained for at least about 365 days after the first dose. In one embodiment, the immune response against C. difficile toxin A and/or toxin B is sustained for at least about 365 days after the second dose. In one embodiment, the immune response against C. difficile toxin A and/or toxin B is sustained for at least about 540 days after the first dose. In one embodiment, the immune response against C. difficile toxin A and/or toxin B is sustained for at least about 540 days after the second dose.

In one embodiment, the second administration is at least 7 days after the first administration and the third administration is at least 30 days after the first administration. In one embodiment, the third administration is at least 30 days after the first administration. In one embodiment, the second administration is at least 7 days after the first administration and the third administration is at least 180 days after the first or second administration. In one embodiment, the third administration is at least 180 days after the first administration. In one embodiment, the composition includes a C. difficile toxoid A and/or a C. difficile toxoid B, each having a purity of at least 90% or greater.

In one embodiment, the composition includes a C. difficile toxoid A and/or a C. difficile toxoid B, in a ratio of about 3:1 to about 1:1. In one embodiment, the composition includes a C. difficile toxoid A and/or a C. difficile toxoid B, in a ratio of 1:1. In one embodiment, the composition includes an adjuvant. In one embodiment, the composition includes an aluminum adjuvant. In one embodiment, the immune response against C. difficile toxin A and/or toxin B is sustained for at least about 60 days. In one embodiment, the immune response against C. difficile toxin A and/or toxin B is sustained for at least about 180 days. In one embodiment, the immune response against C. difficile toxin A and/or toxin B is sustained for at least about 365 days.

In one embodiment, the C. difficile toxoid A is bound to aluminum adjuvant. In one embodiment, the C. difficile toxoid B is bound to aluminum adjuvant. In one embodiment, the C. difficile toxoid A and/or a C. difficile toxoid B are lyophilized.

In one embodiment, the composition induces a toxin A-specific neutralizing antibody concentration that is at least 2-fold higher in the human after receiving the first dose than a toxin A-specific neutralizing antibody concentration in the human prior to receiving the first dose, when measured under identical conditions in a cytotoxicity assay. In one embodiment, the composition induces a toxin A-specific neutralizing antibody concentration that is at least 4-fold higher in the human after receiving the first dose than a toxin A-specific neutralizing antibody concentration in the human prior to receiving the first dose, when measured under identical conditions in a cytotoxicity assay. In one embodiment, the composition induces a toxin A-specific neutralizing antibody concentration that is at least 8-fold higher in the human after receiving the first dose than a toxin A-specific neutralizing antibody concentration in the human prior to receiving the first dose, when measured under identical conditions in a cytotoxicity assay. In one embodiment, the composition induces a toxin A-specific neutralizing antibody concentration that is at least 16-fold higher in the human after receiving the first dose than a toxin A-specific neutralizing antibody concentration in the human prior to receiving the first dose, when measured under identical conditions in a cytotoxicity assay. In one embodiment, the composition induces a toxin A-specific neutralizing antibody concentration that is at least 32-fold higher in the human after receiving the first dose than a toxin A-specific neutralizing antibody concentration in the human prior to receiving the first dose, when measured under identical conditions in a cytotoxicity assay.

In one embodiment, the composition induces a toxin B-specific neutralizing antibody concentration that is at least 2-fold higher in the human after receiving the first dose than a toxin B-specific neutralizing antibody concentration in the human prior to receiving the first dose, when measured under identical conditions in a cytotoxicity assay. In one embodiment, the composition induces a toxin B-specific neutralizing antibody concentration that is at least 4-fold higher in the human after receiving the first dose than a toxin B-specific neutralizing antibody concentration in the human prior to receiving the first dose, when measured under identical conditions in a cytotoxicity assay. In one embodiment, the composition induces a toxin B-specific neutralizing antibody concentration that is at least 8-fold higher in the human after receiving the first dose than a toxin B-specific neutralizing antibody concentration in the human prior to receiving the first dose, when measured under identical conditions in a cytotoxicity assay. In one embodiment, the composition induces a toxin B-specific neutralizing antibody concentration that is at least 16-fold higher in the human after receiving the first dose than a toxin B-specific neutralizing antibody concentration in the human prior to receiving the first dose, when measured under identical conditions in a cytotoxicity assay. In one embodiment, the composition induces a toxin B-specific neutralizing antibody concentration that is at least 32-fold higher in the human after receiving the first dose than a toxin B-specific neutralizing antibody concentration in the human prior to receiving the first dose, when measured under identical conditions in a cytotoxicity assay.

In one embodiment, the composition induces a toxin A-specific neutralizing antibody concentration that is at least 2-fold higher in the human after receiving the second dose than a toxin A-specific neutralizing antibody concentration in the human prior to receiving the first dose, when measured under identical conditions in a cytotoxicity assay. In one embodiment, the composition induces a toxin A-specific neutralizing antibody concentration that is at least 4-fold higher in the human after receiving the second dose than a toxin A-specific neutralizing antibody concentration in the human prior to receiving the first dose, when measured under identical conditions in a cytotoxicity assay. In one embodiment, the composition induces a toxin A-specific neutralizing antibody concentration that is at least 8-fold higher in the human after receiving the second dose than a toxin A-specific neutralizing antibody concentration in the human prior to receiving the first dose, when measured under identical conditions in a cytotoxicity assay. In one embodiment, the composition induces a toxin A-specific neutralizing antibody concentration that is at least 16-fold higher in the human after receiving the second dose than a toxin A-specific neutralizing antibody concentration in the human prior to receiving the first dose, when measured under identical conditions in a cytotoxicity assay. In one embodiment, the composition induces a toxin A-specific neutralizing antibody concentration that is at least 32-fold higher in the human after receiving the second dose than a toxin A-specific neutralizing antibody concentration in the human prior to receiving the first dose, when measured under identical conditions in a cytotoxicity assay.

In one embodiment, the composition induces a toxin B-specific neutralizing antibody concentration that is at least 2-fold higher in the human after receiving the second dose than a toxin B-specific neutralizing antibody concentration in the human prior to receiving the first dose, when measured under identical conditions in a cytotoxicity assay. In one embodiment, the composition induces a toxin B-specific neutralizing antibody concentration that is at least 4-fold higher in the human after receiving the second dose than a toxin B-specific neutralizing antibody concentration in the human prior to receiving the first dose, when measured under identical conditions in a cytotoxicity assay. In one embodiment, the composition induces a toxin B-specific neutralizing antibody concentration that is at least 8-fold higher in the human after receiving the second dose than a toxin B-specific neutralizing antibody concentration in the human prior to receiving the first dose, when measured under identical conditions in a cytotoxicity assay. In one embodiment, the composition induces a toxin B-specific neutralizing antibody concentration that is at least 16-fold higher in the human after receiving the second dose than a toxin B-specific neutralizing antibody concentration in the human prior to receiving the first dose, when measured under identical conditions in a cytotoxicity assay. In one embodiment, the composition induces a toxin B-specific neutralizing antibody concentration that is at least 32-fold higher in the human after receiving the second dose than a toxin B-specific neutralizing antibody concentration in the human prior to receiving the first dose, when measured under identical conditions in a cytotoxicity assay.

In one embodiment, the composition induces a toxin A-specific neutralizing antibody concentration that is at least 2-fold higher in the human after receiving the third dose than a toxin A-specific neutralizing antibody concentration in the human prior to receiving the first dose, when measured under identical conditions in a cytotoxicity assay. In one embodiment, the composition induces a toxin A-specific neutralizing antibody concentration that is at least 4-fold higher in the human after receiving the third dose than a toxin A-specific neutralizing antibody concentration in the human prior to receiving the first dose, when measured under identical conditions in a cytotoxicity assay. In one embodiment, the composition induces a toxin A-specific neutralizing antibody concentration that is at least 8-fold higher in the human after receiving the third dose than a toxin A-specific neutralizing antibody concentration in the human prior to receiving the first dose, when measured under identical conditions in a cytotoxicity assay. In one embodiment, the composition induces a toxin A-specific neutralizing antibody concentration that is at least 16-fold higher in the human after receiving the third dose than a toxin A-specific neutralizing antibody concentration in the human prior to receiving the first dose, when measured under identical conditions in a cytotoxicity assay. In one embodiment, the composition induces a toxin A-specific neutralizing antibody concentration that is at least 32-fold higher in the human after receiving the third dose than a toxin A-specific neutralizing antibody concentration in the human prior to receiving the first dose, when measured under identical conditions in a cytotoxicity assay.

In one embodiment, the composition induces a toxin B-specific neutralizing antibody concentration that is at least 2-fold higher in the human after receiving the third dose than a toxin B-specific neutralizing antibody concentration in the human prior to receiving the first dose, when measured under identical conditions in a cytotoxicity assay. In one embodiment, the composition induces a toxin B-specific neutralizing antibody concentration that is at least 4-fold higher in the human after receiving the third dose than a toxin B-specific neutralizing antibody concentration in the human prior to receiving the first dose, when measured under identical conditions in a cytotoxicity assay. In one embodiment, the composition induces a toxin B-specific neutralizing antibody concentration that is at least 8-fold higher in the human after receiving the third dose than a toxin B-specific neutralizing antibody concentration in the human prior to receiving the first dose, when measured under identical conditions in a cytotoxicity assay. In one embodiment, the composition induces a toxin B-specific neutralizing antibody concentration that is at least 16-fold higher in the human after receiving the third dose than a toxin B-specific neutralizing antibody concentration in the human prior to receiving the first dose, when measured under identical conditions in a cytotoxicity assay. In one embodiment, the composition induces a toxin B-specific neutralizing antibody concentration that is at least 32-fold higher in the human after receiving the third dose than a toxin B-specific neutralizing antibody concentration in the human prior to receiving the first dose, when measured under identical conditions in a cytotoxicity assay.

In one embodiment, the composition induces a toxin A-specific neutralizing antibody concentration that is at least 2-fold higher in the human after receiving the first dose when measured about 7, 30, 60, 90, 120, 365, or 540 days after the first dose than a toxin A-specific neutralizing antibody concentration in the human prior to receiving the first dose, when measured under identical conditions in a cytotoxicity assay. In one embodiment, the composition induces a toxin A-specific neutralizing antibody concentration that is at least 2-fold higher in the human after receiving the second dose when measured about 7, 30, 60, 90, 120, 365, or 540 days after the second dose than a toxin A-specific neutralizing antibody concentration in the human prior to receiving the first dose, when measured under identical conditions in a cytotoxicity assay. In one embodiment, the composition induces a toxin A-specific neutralizing antibody concentration that is at least 2-fold higher in the human after receiving the third dose when measured about 7, 30, 60, 90, 120, 365, or 540 days after the third dose than a toxin A-specific neutralizing antibody concentration in the human prior to receiving the first dose, when measured under identical conditions in a cytotoxicity assay. In one embodiment, the composition induces a toxin B-specific neutralizing antibody concentration that is at least 2-fold higher in the human after receiving the first dose when measured about 7, 30, 60, 90, 120, 365, or 540 days after the first dose than a toxin B-specific neutralizing antibody concentration in the human prior to receiving the first dose, when measured under identical conditions in a cytotoxicity assay. In one embodiment, the composition induces a toxin B-specific neutralizing antibody concentration that is at least 2-fold higher in the human after receiving the second dose when measured about 7, 30, 60, 90, 120, 365, or 540 days after the second dose than a toxin B-specific neutralizing antibody concentration in the human prior to receiving the first dose, when measured under identical conditions in a cytotoxicity assay. In one embodiment, the composition induces a toxin B-specific neutralizing antibody concentration that is at least 2-fold higher in the human after receiving the third dose when measured on about any one of 7, 30, 60, 90, 120, 365, or 540 days after the third dose than a toxin B-specific neutralizing antibody concentration in the human prior to receiving the first dose, when measured under identical conditions in a cytotoxicity assay.

In one embodiment, the composition induces a toxin B-specific neutralizing antibody concentration in the human after receiving the third dose when measured on about any one of 7, 30, 60, 90, 120, 365, or 540 days after the third dose than a toxin B-specific neutralizing antibody concentration in the human prior to receiving the first dose, when measured under identical conditions in a cytotoxicity assay.

In one embodiment, the human is seronegative for toxin B. In one embodiment, the human is seronegative for toxin A. In one embodiment, the human is seronegative for toxin A and toxin B. In one embodiment, the human is seropositive for toxin B. In one embodiment, the human is seropositive for toxin A. In one embodiment, the human is seropositive for toxin A and toxin B.

In one embodiment, the toxoid is a polypeptide that includes SEQ ID NO: 4, wherein the methionine is absent. In one embodiment, the toxoid is a polypeptide that includes SEQ ID NO: 6, wherein the methionine is absent. In one embodiment, the toxoid includes a formaldehyde-contacted C. difficile toxin A. In one embodiment, the toxoid includes a formaldehyde-contacted C. difficile toxin B. In another embodiment, the toxoid is not a formaldehyde-contacted polypeptide.

In one embodiment, the infection is from a C. difficile Ribotype 002. In one embodiment, the infection is from a C. difficile Ribotype 003. In one embodiment, the infection is from a C. difficile Ribotype 004. In one embodiment, the infection is from a C. difficile Ribotype 012. In one embodiment, the infection is from a C. difficile Ribotype 015. In one embodiment, the infection is from a C. difficile Ribotype 017. In one embodiment, the infection is from a C. difficile Ribotype 020. In one embodiment, the infection is from a C. difficile Ribotype 023. In one embodiment, the infection is from a C. difficile Ribotype 027. In one embodiment, the infection is from a C. difficile Ribotype 029. In one embodiment, the infection is from a C. difficile Ribotype 046. In one embodiment, the infection is from a C. difficile Ribotype 053. In one embodiment, the infection is from a C. difficile Ribotype 059. In one embodiment, the infection is from a C. difficile Ribotype 070. In one embodiment, the infection is from a C. difficile Ribotype 075. In one embodiment, the infection is from a C. difficile Ribotype 078. In one embodiment, the infection is from a C. difficile Ribotype 081. In one embodiment, the infection is from a C. difficile Ribotype 087. In one embodiment, the infection is from a C. difficile Ribotype 106. In one embodiment, the infection is from a C. difficile Ribotype 117. In one embodiment, the infection is from a C. difficile Ribotype 126. In one embodiment, the infection is from a C. difficile Ribotype 131. In one embodiment, the infection is from a C. difficile Ribotype 154. In one embodiment, the infection is from a C. difficile Toxinotype 0. In one embodiment, the infection is from a C. difficile Toxinotype I. In one embodiment, the infection is from a C. difficile Toxinotype VIII. In one embodiment, the infection is from a C. difficile Toxinotype IV. In one embodiment, the infection is from a C. difficile Toxinotype III. In one embodiment, the infection is from a C. difficile Toxinotype XIII. In one embodiment, the infection is from a C. difficile Toxinotype V.

### BRIEF DESCRIPTION OF THE DRAWINGS

FIG. 1A-D: FIG. 1A-B—Proportion of subjects above threshold*—Months 0, 1, 6 regimen (Evaluable immunogenicity population). Asterisk (*)≥219 neutralization units/mL for toxin A (FIG. 1A) and ≥2586 neutralization units/mL for toxin B (FIG. 1B). Sera were not collected on days 8 & 15 & month 4 in the months regimen. FIG. 1C and FIG. 1D—Proportion of subjects above threshold*—Days 1, 8, 30 regimen (Evaluable immunogenicity population). Asterisk (*)≥219 neutralization units/mL for toxin A (FIG. 1C) and ≥2586 neutralization units/mL for toxin B (FIG. 1D). Sera were not collected on days 8 & 15 & month 4 in the months regimen.

FIG. 2A-2D—FIG. 2A-B Geometric mean concentrations—Months 0, 1, 6 regimen (Evaluable immunogenicity population). Dotted line (--) represents 219 neutralization units/mL for toxin A (FIG. 2A) and 2586 neutralization units/mL for toxin B (FIG. 2B). Sera were not collected on days 8 & 15 & month 4 in the months regimen. FIG. 2C-D Geometric mean concentrations—Day 1, 8, 30 regimen (Evaluable immunogenicity population). Dotted line (--) represents 219 neutralization units/mL for toxin A (FIG. 2C) and 2586 neutralization units/mL for toxin B (FIG. 2D). Sera were not collected on days 8 & 15 & month 4 in the months regimen.

FIG. 3A-B—Overview of adverse events (Safety population). Top panel relates to the Month 0, 1, 6 regimen (FIG. 3A). Bottom panel relates to Days 1, 8, 30 regimen (FIG. 3B). Note protocol-specified adverse event reporting periods: non-serious up to 1 month post-dose 3, serious up to 6 months post-dose 3 (i.e., reporting periods were 5 months longer for the months regimen).

FIG. 4A-B—Geometric mean concentrations—Toxin A (Evaluable immunogenicity population). Top panel relates to the Month 0, 1, 6 regimen (FIG. 4A). Bottom panel relates to Days 1, 8, 30 regimen (FIG. 4B). Dashed line represents 219 neutralization units/mL for toxin A; Sera were not collected on days 8 & 15 & month 4 in the months regimen; Sera were not collected on month 6 & day 187 in the days regimen.

FIG. 5A-B—Geometric mean concentrations—Toxin B (Evaluable immunogenicity population). Top panel relates to the Month 0, 1, 6 regimen (FIG. 5A). Bottom panel relates to Days 1, 8, 30 regimen. Dashed line represents 2586 neutralization units/mL for toxin B (FIG. 5B). Sera were not collected on days 8 & 15 & month 4 in the months regimen; Sera were not collected on month 6 & day 187 in the days regimen.

FIG. 6A-B—Geometric mean concentrations by baseline serostatus—Toxin A (Evaluable immunogenicity population). Top panel relates to the Month 0, 1, 6 regimen (FIG. 6A). Bottom panel relates to Days 1, 8, 30 regimen (FIG. 6B). Dashed line represents 219 neutralization units/mL for toxin A; Sera were not collected on days 8 & 15 & month 4 in the months regimen; Sera were not collected on month 6 & day 187 in the days regimen.

FIG. 7A-B—Geometric mean concentrations by baseline serostatus—Toxin B (Evaluable immunogenicity population). Top panel relates to the Month 0, 1, 6 regimen (FIG. 7A). Bottom panel relates to Days 1, 8, 30 regimen (FIG. 7B). Dashed line represents 2586 neutralization units/mL for toxin B. Sera were not collected on days 8 & 15 & month 4 in the months regimen; Sera were not collected on month 6 & day 187 in the days regimen.

FIG. 8A-B—Geometric mean concentrations by baseline serostatus—(Month Regimen)—toxin A & toxin B (Evaluable immunogenicity population). Top panel relates to Toxin A (FIG. 8A). Dashed line represents 219 neutralization units/mL for toxin A. Bottom panel relates to Toxin B (FIG. 8B). Dashed line represents 2586 neutralization units/mL for toxin B. Sera were not collected on days 8 & 15 & month 4 in the months regimen; Sera were not collected on month 6 & day 187 in the days regimen.

FIG. 9A-B—Geometric mean concentrations by baseline serostatus—(Day Regimen)—toxin A & toxin B (Evaluable immunogenicity population). Top panel relates to Toxin A (FIG. 9A). Dashed line represents 219 neutralization units/mL for toxin A. Bottom panel relates to Toxin B (FIG. 9B). Dashed line represents 2586 neutralization units/mL for toxin B. Sera were not collected on days 8 & 15 & month 4 in the months regimen; Sera were not collected on month 6 & day 187 in the days regimen.

### SEQUENCE IDENTIFIERS

SEQ ID NO: 1 sets forth the amino acid sequence for wild-type C. difficile 630 toxin A (TcdA).

SEQ ID NO: 2 sets forth the amino acid sequence for wild-type C. difficile 630 toxin B (TcdB).

SEQ ID NO: 3 sets forth the amino acid sequence for a mutant TcdA having a mutation at positions 285 and 287, as compared to SEQ ID NO: 1.

SEQ ID NO: 4 sets forth the amino acid sequence for a mutant TcdA having a mutation at positions 285, 287, and 700, as compared to SEQ ID NO: 1.

SEQ ID NO: 5 sets forth the amino acid sequence for a mutant TcdB having a mutation at positions 286 and 288, as compared to SEQ ID NO: 2.

SEQ ID NO: 6 sets forth the amino acid sequence for a mutant TcdB having a mutation at positions 286, 288, and 698, as compared to SEQ ID NO: 2.

SEQ ID NO: 7 sets forth the amino acid sequence for a mutant TcdA having a mutation at positions 269, 272, 285, 287, 460, 462, and 700, as compared to SEQ ID NO: 1

SEQ ID NO: 8 sets forth the amino acid sequence for a mutant TcdB having a mutation at positions 270, 273, 286, 288, 461, 463, and 698, as compared to SEQ ID NO: 2

SEQ ID NO: 9 sets forth a DNA sequence encoding a wild-type C. difficile 630 toxin A (TcdA).

SEQ ID NO: 10 sets forth a DNA sequence encoding a wild-type C. difficile 630 toxin B (TcdB).

SEQ ID NO: 11 sets forth a DNA sequence encoding SEQ ID NO: 3

SEQ ID NO: 12 sets forth a DNA sequence encoding SEQ ID NO: 4

SEQ ID NO: 13 sets forth a DNA sequence encoding SEQ ID NO: 5

SEQ ID NO: 14 sets forth a DNA sequence encoding SEQ ID NO: 6

SEQ ID NO: 15 sets forth the amino acid sequence for wild-type C. difficile R20291 TcdA.

SEQ ID NO: 16 sets forth a DNA sequence encoding SEQ ID NO: 15.

SEQ ID NO: 17 sets forth the amino acid sequence for wild-type C. difficile CD196 TcdA.

SEQ ID NO: 18 sets forth a DNA sequence encoding SEQ ID NO: 17.

SEQ ID NO: 19 sets forth the amino acid sequence for wild-type C. difficile VP110463 TcdA.

SEQ ID NO: 20 sets forth a DNA sequence encoding SEQ ID NO: 19.

SEQ ID NO: 21 sets forth the amino acid sequence for wild-type C. difficile R20291 TcdB.

SEQ ID NO: 22 sets forth a DNA sequence encoding SEQ ID NO: 21.

SEQ ID NO: 23 sets forth the amino acid sequence for wild-type C. difficile CD196 TcdB.

SEQ ID NO: 24 sets forth a DNA sequence encoding SEQ ID NO: 23.

SEQ ID NO: 25 sets forth the amino acid sequence for wild-type C. difficile VP110463 TcdB.

SEQ ID NO: 26 sets forth a DNA sequence encoding SEQ ID NO: 25.

SEQ ID NO: 27 sets forth a DNA sequence of a pathogenicity locus of wild-type C. difficile VP110463.

SEQ ID NO: 28 sets forth the amino acid sequence for residues 101 to 293 of SEQ ID NO: 1.

SEQ ID NO: 29 sets forth the amino acid sequence for residues 1 to 542 of SEQ ID NO: 1.

SEQ ID NO: 30 sets forth the amino acid sequence for residues 101 to 293 of SEQ ID NO: 2.

SEQ ID NO: 31 sets forth the amino acid sequence for residues 1 to 543 of SEQ ID NO: 2.

SEQ ID NO: 32 sets forth the amino acid sequence for residues 543 to 809 of SEQ ID NO: 1.

SEQ ID NO: 33 sets forth the amino acid sequence for residues 544 to 767 of SEQ ID NO: 2.

SEQ ID NO: 34 sets forth the amino acid sequence for a mutant TcdA, wherein residues 101, 269, 272, 285, 287, 460, 462, 541, 542, 543, 589, 655, and 700 may be any amino acid.

SEQ ID NO: 35 sets forth the amino acid sequence for a mutant TcdB, wherein 102, 270, 273, 286, 288, 384, 461, 463, 520, 543, 544, 587, 600, 653, 698, and 751 may be any amino acid.

SEQ ID NO: 36 sets forth the amino acid sequence for the variable light chain of a neutralizing antibody of C. difficile TcdA (A3-25 mAb).

SEQ ID NO: 37 sets forth the amino acid sequence for the variable heavy chain of a neutralizing antibody of C. difficile TcdA (A3-25 mAb).

SEQ ID NO: 38 sets forth the amino acid sequence for CDR1 of the variable light chain of neutralizing antibody of C. difficile TcdA (A3-25 mAb).

SEQ ID NO: 39 sets forth the amino acid sequence for CDR2 of the variable light chain of neutralizing antibody of C. difficile TcdA (A3-25 mAb).

SEQ ID NO: 40 sets forth the amino acid sequence for CDR3 of the variable light chain of neutralizing antibody of C. difficile TcdA (A3-25 mAb).

SEQ ID NO: 41 sets forth the amino acid sequence for CDR1 of the variable heavy chain of neutralizing antibody of C. difficile TcdA (A3-25 mAb).

SEQ ID NO: 42 sets forth the amino acid sequence for CDR2 of the variable heavy chain of neutralizing antibody of C. difficile TcdA (A3-25 mAb).

SEQ ID NO: 43 sets forth the amino acid sequence for CDR3 of the variable heavy chain of neutralizing antibody of C. difficile TcdA (A3-25 mAb).

SEQ ID NO: 44 sets forth a DNA sequence encoding SEQ ID NO: 3.

SEQ ID NO: 45 sets forth a DNA sequence encoding SEQ ID NO: 4.

SEQ ID NO: 46 sets forth a DNA sequence encoding SEQ ID NO: 5.

SEQ ID NO: 47 sets forth a DNA sequence encoding SEQ ID NO: 6.

SEQ ID NO: 48 sets forth the nucleotide sequence of immunostimulatory oligonucleotide ODN CpG 24555.

SEQ ID NO: 49 sets forth the amino acid sequence for the variable heavy chain of a C. difficile TcdB neutralizing antibody (B8-26 mAb).

SEQ ID NO: 50 sets forth the amino acid sequence for the signal peptide of the variable heavy chain of a C. difficile TcdB neutralizing antibody (B8-26 mAb).

SEQ ID NO: 51 sets forth the amino acid sequence for CDR1 of the variable heavy chain of a C. difficile TcdB neutralizing antibody (B8-26 mAb).

SEQ ID NO: 52 sets forth the amino acid sequence for CDR2 of the variable heavy chain of a C. difficile TcdB neutralizing antibody (B8-26 mAb).

SEQ ID NO: 53 sets forth the amino acid sequence for CDR3 of the variable heavy chain of a C. difficile TcdB neutralizing antibody (B8-26 mAb).

SEQ ID NO: 54 sets forth the amino acid sequence for the constant region of the variable heavy chain of a C. difficile TcdB neutralizing antibody (B8-26 mAb).

SEQ ID NO: 55 sets forth the amino acid sequence for the variable light chain of a C. difficile TcdB neutralizing antibody (B8-26 mAb).

SEQ ID NO: 56 sets forth the amino acid sequence for the signal peptide of the variable light chain of a C. difficile TcdB neutralizing antibody (B8-26 mAb).

SEQ ID NO: 57 sets forth the amino acid sequence for CDR1 of the variable light chain of a C. difficile TcdB neutralizing antibody (B8-26 mAb).

SEQ ID NO: 58 sets forth the amino acid sequence for CDR2 of the variable light chain of a C. difficile TcdB neutralizing antibody (B8-26 mAb).

SEQ ID NO: 59 sets forth the amino acid sequence for CDR3 of the variable light chain of a C. difficile TcdB neutralizing antibody (B8-26 mAb).

SEQ ID NO: 60 sets forth the amino acid sequence for the variable heavy chain of a C. difficile TcdB neutralizing antibody (B59-3 mAb).

SEQ ID NO: 61 sets forth the amino acid sequence for the signal peptide of the variable heavy chain of a C. difficile TcdB neutralizing antibody (B59-3 mAb).

SEQ ID NO: 62 sets forth the amino acid sequence for CDR1 of the variable heavy chain of a C. difficile TcdB neutralizing antibody (B59-3 mAb).

SEQ ID NO: 63 sets forth the amino acid sequence for CDR2 of the variable heavy chain of a C. difficile TcdB neutralizing antibody (B59-3 mAb).

SEQ ID NO: 64 sets forth the amino acid sequence for CDR3 of the variable heavy chain of a C. difficile TcdB neutralizing antibody (B59-3 mAb).

SEQ ID NO: 65 sets forth the amino acid sequence for the constant region of the variable heavy chain of a C. difficile TcdB neutralizing antibody (B59-3 mAb).

SEQ ID NO: 66 sets forth the amino acid sequence for the variable light chain of a C. difficile TcdB neutralizing antibody (B59-3 mAb).

SEQ ID NO: 67 sets forth the amino acid sequence for the signal peptide of the variable light chain of a C. difficile TcdB neutralizing antibody (B59-3 mAb).

SEQ ID NO: 68 sets forth the amino acid sequence for CDR1 of the variable light chain of a C. difficile TcdB neutralizing antibody (B59-3 mAb).

SEQ ID NO: 69 sets forth the amino acid sequence for CDR2 of the variable light chain of a C. difficile TcdB neutralizing antibody (B59-3 mAb).

SEQ ID NO: 70 sets forth the amino acid sequence for CDR3 of the variable light chain of a C. difficile TcdB neutralizing antibody (B59-3 mAb).

SEQ ID NO: 71 sets forth the amino acid sequence for the variable heavy chain of a C. difficile TcdB neutralizing antibody (B9-30 mAb).

SEQ ID NO: 72 sets forth the amino acid sequence for the signal peptide of the variable heavy chain of a C. difficile TcdB neutralizing antibody (B9-30 mAb).

SEQ ID NO: 73 sets forth the amino acid sequence for CDR1 of the variable heavy chain of a C. difficile TcdB neutralizing antibody (B9-30 mAb).

SEQ ID NO: 74 sets forth the amino acid sequence for CDR2 of the variable heavy chain of a C. difficile TcdB neutralizing antibody (B9-30 mAb).

SEQ ID NO: 75 sets forth the amino acid sequence for CDR3 of the variable heavy chain of a C. difficile TcdB neutralizing antibody (B9-30 mAb).

SEQ ID NO: 76 sets forth the amino acid sequence for the constant region of the variable heavy chain of a C. difficile TcdB neutralizing antibody (B9-30 mAb).

SEQ ID NO: 77 sets forth the amino acid sequence for the variable light chain of a C. difficile TcdB neutralizing antibody (B9-30 mAb).

SEQ ID NO: 78 sets forth the amino acid sequence for the signal peptide of the variable light chain of a C. difficile TcdB neutralizing antibody (B9-30 mAb).

SEQ ID NO: 79 sets forth the amino acid sequence for CDR1 of the variable light chain of a C. difficile TcdB neutralizing antibody (B9-30 mAb).

SEQ ID NO: 80 sets forth the amino acid sequence for CDR2 of the variable light chain of a C. difficile TcdB neutralizing antibody (B9-30 mAb).

SEQ ID NO: 81 sets forth the amino acid sequence for CDR3 of the variable light chain of a C. difficile TcdB neutralizing antibody (B9-30 mAb).

SEQ ID NO: 82 sets forth the amino acid sequence for a mutant TcdB, wherein a residue at positions 102, 270, 273, 286, 288, 384, 461, 463, 520, 543, 544, 587, 600, 653, 698, and 751 may be any amino acid.

SEQ ID NO: 83 sets forth the amino acid sequence for a mutant TcdA having a mutation at positions 269, 272, 285, 287, 460, 462, and 700, as compared to SEQ ID NO: 1, wherein the methionine at position 1 is absent.

SEQ ID NO: 84 sets forth the amino acid sequence for a mutant C. difficile toxin A having a mutation at positions 285, 287, and 700, as compared to SEQ ID NO: 1, wherein the methionine at position 1 is absent.

SEQ ID NO: 85 sets forth the amino acid sequence for a mutant C. difficile toxin B having a mutation at positions 270, 273, 286, 288, 461, 463, and 698, as compared to SEQ ID NO: 2, wherein the methionine at position 1 is absent.

SEQ ID NO: 86 sets forth the amino acid sequence for a mutant C. difficile toxin B having a mutation at positions 286, 288, and 698, as compared to SEQ ID NO: 2, wherein the methionine at position 1 is absent.

SEQ ID NO: 87 sets forth the amino acid sequence for wild-type C. difficile 2004013 TcdA.

SEQ ID NO: 88 sets forth the amino acid sequence for wild-type C. difficile 2004111 TcdA.

SEQ ID NO: 89 sets forth the amino acid sequence for wild-type C. difficile 2004118 TcdA.

SEQ ID NO: 90 sets forth the amino acid sequence for wild-type C. difficile 2004205 TcdA.

SEQ ID NO: 91 sets forth the amino acid sequence for wild-type C. difficile 2004206 TcdA.

SEQ ID NO: 92 sets forth the amino acid sequence for wild-type C. difficile 2005022 TcdA.

SEQ ID NO: 93 sets forth the amino acid sequence for wild-type C. difficile 2005088 TcdA.

SEQ ID NO: 94 sets forth the amino acid sequence for wild-type C. difficile 2005283 TcdA.

SEQ ID NO: 95 sets forth the amino acid sequence for wild-type C. difficile 2005325 TcdA.

SEQ ID NO: 96 sets forth the amino acid sequence for wild-type C. difficile 2005359 TcdA.

SEQ ID NO: 97 sets forth the amino acid sequence for wild-type C. difficile 2006017 TcdA.

SEQ ID NO: 98 sets forth the amino acid sequence for wild-type C. difficile 2007070 TcdA.

SEQ ID NO: 99 sets forth the amino acid sequence for wild-type C. difficile 2007217 TcdA.

SEQ ID NO: 100 sets forth the amino acid sequence for wild-type C. difficile 2007302 TcdA.

SEQ ID NO: 101 sets forth the amino acid sequence for wild-type C. difficile 2007816 TcdA.

SEQ ID NO: 102 sets forth the amino acid sequence for wild-type C. difficile 2007838 TcdA.

SEQ ID NO: 103 sets forth the amino acid sequence for wild-type C. difficile 2007858 TcdA.

SEQ ID NO: 104 sets forth the amino acid sequence for wild-type C. difficile 2007886 TcdA.

SEQ ID NO: 105 sets forth the amino acid sequence for wild-type C. difficile 2008222 TcdA.

SEQ ID NO: 106 sets forth the amino acid sequence for wild-type C. difficile 2009078 TcdA.

SEQ ID NO: 107 sets forth the amino acid sequence for wild-type C. difficile 2009087 TcdA.

SEQ ID NO: 108 sets forth the amino acid sequence for wild-type C. difficile 2009141 TcdA.

SEQ ID NO: 109 sets forth the amino acid sequence for wild-type C. difficile 2009292 TcdA.

SEQ ID NO: 110 sets forth the amino acid sequence for wild-type C. difficile 2004013 TcdB.

SEQ ID NO: 111 sets forth the amino acid sequence for wild-type C. difficile 2004111 TcdB.

SEQ ID NO: 112 sets forth the amino acid sequence for wild-type C. difficile 2004118 TcdB.

SEQ ID NO: 113 sets forth the amino acid sequence for wild-type C. difficile 2004205 TcdB.

SEQ ID NO: 114 sets forth the amino acid sequence for wild-type C. difficile 2004206 TcdB.

SEQ ID NO: 115 sets forth the amino acid sequence for wild-type C. difficile 2005022 TcdB.

SEQ ID NO: 116 sets forth the amino acid sequence for wild-type C. difficile 2005088 TcdB.

SEQ ID NO: 117 sets forth the amino acid sequence for wild-type C. difficile 2005283 TcdB.

SEQ ID NO: 118 sets forth the amino acid sequence for wild-type C. difficile 2005325 TcdB.

SEQ ID NO: 119 sets forth the amino acid sequence for wild-type C. difficile 2005359 TcdB.

SEQ ID NO: 120 sets forth the amino acid sequence for wild-type C. difficile 2006017 TcdB.

SEQ ID NO: 121 sets forth the amino acid sequence for wild-type C. difficile 2006376 TcdB.

SEQ ID NO: 122 sets forth the amino acid sequence for wild-type C. difficile 2007070 TcdB.

SEQ ID NO: 123 sets forth the amino acid sequence for wild-type C. difficile 2007217 TcdB.

SEQ ID NO: 124 sets forth the amino acid sequence for wild-type C. difficile 2007302 TcdB.

SEQ ID NO: 125 sets forth the amino acid sequence for wild-type C. difficile 2007816 TcdB.

SEQ ID NO: 126 sets forth the amino acid sequence for wild-type C. difficile 2007838 TcdB.

SEQ ID NO: 127 sets forth the amino acid sequence for wild-type C. difficile 2007858 TcdB.

SEQ ID NO: 128 sets forth the amino acid sequence for wild-type C. difficile 2007886 TcdB.

SEQ ID NO: 129 sets forth the amino acid sequence for wild-type C. difficile 2008222 TcdB.

SEQ ID NO: 130 sets forth the amino acid sequence for wild-type C. difficile 2009078 TcdB.

SEQ ID NO: 131 sets forth the amino acid sequence for wild-type C. difficile 2009087 TcdB.

SEQ ID NO: 132 sets forth the amino acid sequence for wild-type C. difficile 2009141 TcdB.

SEQ ID NO: 133 sets forth the amino acid sequence for wild-type C. difficile 2009292 TcdB.

SEQ ID NO: 134 sets forth the amino acid sequence for wild-type C. difficile 014 TcdA.

SEQ ID NO: 135 sets forth the amino acid sequence for wild-type C. difficile 015 TcdA.

SEQ ID NO: 136 sets forth the amino acid sequence for wild-type C. difficile 020 TcdA.

SEQ ID NO: 137 sets forth the amino acid sequence for wild-type C. difficile 023 TcdA.

SEQ ID NO: 138 sets forth the amino acid sequence for wild-type C. difficile 027 TcdA.

SEQ ID NO: 139 sets forth the amino acid sequence for wild-type C. difficile 029 TcdA.

SEQ ID NO: 140 sets forth the amino acid sequence for wild-type C. difficile 046 TcdA.

SEQ ID NO: 141 sets forth the amino acid sequence for wild-type C. difficile 014 TcdB.

SEQ ID NO: 142 sets forth the amino acid sequence for wild-type C. difficile 015 TcdB.

SEQ ID NO: 143 sets forth the amino acid sequence for wild-type C. difficile 020 TcdB.

SEQ ID NO: 144 sets forth the amino acid sequence for wild-type C. difficile 023 TcdB.

SEQ ID NO: 145 sets forth the amino acid sequence for wild-type C. difficile 027 TcdB.

SEQ ID NO: 146 sets forth the amino acid sequence for wild-type C. difficile 029 TcdB.

SEQ ID NO: 147 sets forth the amino acid sequence for wild-type C. difficile 046 TcdB.

SEQ ID NO: 148 sets forth the amino acid sequence for wild-type C. difficile 001 TcdA.

SEQ ID NO: 149 sets forth the amino acid sequence for wild-type C. difficile 002 TcdA.

SEQ ID NO: 150 sets forth the amino acid sequence for wild-type C. difficile 003 TcdA.

SEQ ID NO: 151 sets forth the amino acid sequence for wild-type C. difficile 004 TcdA.

SEQ ID NO: 152 sets forth the amino acid sequence for wild-type C. difficile 070 TcdA.

SEQ ID NO: 153 sets forth the amino acid sequence for wild-type C. difficile 075 TcdA.

SEQ ID NO: 154 sets forth the amino acid sequence for wild-type C. difficile 077 TcdA.

SEQ ID NO: 155 sets forth the amino acid sequence for wild-type C. difficile 081 TcdA.

SEQ ID NO: 156 sets forth the amino acid sequence for wild-type C. difficile 117 TcdA.

SEQ ID NO: 157 sets forth the amino acid sequence for wild-type C. difficile 131 TcdA.

SEQ ID NO: 158 sets forth the amino acid sequence for wild-type C. difficile 001 TcdB.

SEQ ID NO: 159 sets forth the amino acid sequence for wild-type C. difficile 002 TcdB.

SEQ ID NO: 160 sets forth the amino acid sequence for wild-type C. difficile 003 TcdB.

SEQ ID NO: 161 sets forth the amino acid sequence for wild-type C. difficile 004 TcdB.

SEQ ID NO: 162 sets forth the amino acid sequence for wild-type C. difficile 070 TcdB.

SEQ ID NO: 163 sets forth the amino acid sequence for wild-type C. difficile 075 TcdB.

SEQ ID NO: 164 sets forth the amino acid sequence for wild-type C. difficile 077 TcdB.

SEQ ID NO: 165 sets forth the amino acid sequence for wild-type C. difficile 081 TcdB.

SEQ ID NO: 166 sets forth the amino acid sequence for wild-type C. difficile 117 TcdB.

SEQ ID NO: 167 sets forth the amino acid sequence for wild-type C. difficile 131 TcdB.

SEQ ID NO: 168 sets forth the amino acid sequence for wild-type C. difficile 053 TcdA.

SEQ ID NO: 169 sets forth the amino acid sequence for wild-type C. difficile 078 TcdA.

SEQ ID NO: 170 sets forth the amino acid sequence for wild-type C. difficile 087 TcdA.

SEQ ID NO: 171 sets forth the amino acid sequence for wild-type C. difficile 095 TcdA.

SEQ ID NO: 172 sets forth the amino acid sequence for wild-type C. difficile 126 TcdA.

SEQ ID NO: 173 sets forth the amino acid sequence for wild-type C. difficile 053 TcdB.

SEQ ID NO: 174 sets forth the amino acid sequence for wild-type C. difficile 078 TcdB.

SEQ ID NO: 175 sets forth the amino acid sequence for wild-type C. difficile 087 TcdB.

SEQ ID NO: 176 sets forth the amino acid sequence for wild-type C. difficile 095 TcdB.

SEQ ID NO: 177 sets forth the amino acid sequence for wild-type C. difficile 126 TcdB.

SEQ ID NO: 178 sets forth the amino acid sequence for wild-type C. difficile 059 TcdA.

SEQ ID NO: 179 sets forth the amino acid sequence for wild-type C. difficile 059 TcdB.

SEQ ID NO: 180 sets forth the amino acid sequence for wild-type C. difficile 106 TcdA.

SEQ ID NO: 181 sets forth the amino acid sequence for wild-type C. difficile 106 TcdB.

SEQ ID NO: 182 sets forth the amino acid sequence for wild-type C. difficile 017 TcdB.

SEQ ID NO: 183 sets forth the amino acid sequence for a mutant TcdA having a mutation at positions 285, 287, 700, 972, and 978 as compared to SEQ ID NO: 1.

SEQ ID NO: 184 sets forth the amino acid sequence for a mutant TcdB having a mutation at positions 286, 288, 698, 970, and 976 as compared to SEQ ID NO: 2.

SEQ ID NO: 185 through SEQ ID NO: 195 each set forth the amino acid sequence for an exemplary mutant toxin.

SEQ ID NO: 196 through SEQ ID NO: 212 each set forth the amino acid sequence for an exemplary mutant toxin A.

SEQ ID NO: 213 through SEQ ID NO: 222 each set forth the amino acid sequence for an exemplary mutant toxin B.

SEQ ID NO: 223 through SEQ ID NO: 236 each set forth the amino acid sequence for an exemplary mutant toxin A.

SEQ ID NO: 237 through SEQ ID NO: 243 each set forth the amino acid sequence for an exemplary mutant toxin B.

SEQ ID NO: 244 through SEQ ID NO: 245 each set forth the amino acid sequence for an exemplary mutant toxin A.

SEQ ID NO: 246 through SEQ ID NO: 249 each set forth the amino acid sequence for an exemplary mutant toxin B.

SEQ ID NO: 250 through SEQ ID NO: 253 each set forth the amino acid sequence for an exemplary mutant toxin A.

SEQ ID NO: 254 sets forth the amino acid sequence for an exemplary mutant toxin.

SEQ ID NO: 255 through SEQ ID NO: 263 each set forth the amino acid sequence for an exemplary mutant toxin A.

SEQ ID NO: 264 through SEQ ID NO: 269 each set forth the amino acid sequence for an exemplary mutant toxin B.

SEQ ID NO: 270 through SEQ ID NO: 275 each set forth the amino acid sequence for an exemplary mutant toxin.

SEQ ID NO: 276 through SEQ ID NO: 323 each set forth the amino acid sequence for an exemplary mutant toxin A.

SEQ ID NO: 324 through SEQ ID NO: 373 each set forth the amino acid sequence for an exemplary mutant toxin B.

SEQ ID NO: 374 through SEQ ID NO: 421 each set forth the amino acid sequence for an exemplary mutant toxin A.

SEQ ID NO: 422 through SEQ ID NO: 471 each set forth the amino acid sequence for an exemplary mutant toxin B.

SEQ ID NO: 472 through SEQ ID NO: 519 each set forth the amino acid sequence for an exemplary mutant toxin A.

SEQ ID NO: 568 through SEQ ID NO: 615 each set forth the amino acid sequence for an exemplary mutant toxin B.

SEQ ID NO: 520 through SEQ ID NO: 567 each set forth the amino acid sequence for an exemplary mutant toxin A.

SEQ ID NO: 616 through SEQ ID NO: 663 each set forth the amino acid sequence for an exemplary mutant toxin B.

SEQ ID NO: 664 through SEQ ID NO: 711 each set forth the amino acid sequence for an exemplary mutant toxin A.

SEQ ID NO: 712 through SEQ ID NO: 761 each set forth the amino acid sequence for an exemplary mutant toxin B.

### DETAILED DESCRIPTION OF THE INVENTION

The inventors surprisingly discovered highly immunogenic and well tolerated compositions and methods that may be used to treat, ameliorate, reduce the risk of, and/or prevent infection by C. difficile. More specifically, for example, the inventors discovered, among other things, the immunogenicity of two antigen dose levels (100 μg and 200 μg total toxoid) of C. difficile vaccine when administered as a 3-dose regimen (Days 1, 8, and 30) to healthy adults aged 65 to 85 years, as measured by C. difficile toxin A- and toxin B-specific neutralizing antibody levels at Day 37 (7 days after Dose 3). The inventors also discovered, among other things, the immunogenicity of 2 antigen dose levels (100 μg and 200 μg total toxoid) of C. difficile vaccine when administered as a 3-dose regimen (Months 0, 1, and 6) to healthy adults aged 65 to 85 years, as measured by C. difficile toxin A- and toxin B-specific neutralizing antibody levels at Month 7 (1 month after Dose 3). The inventors further discovered, among other things, the immunogenicity of 2 antigen dose levels (100 μg and 200 μg total toxoid) of C. difficile vaccine when administered in a 3-dose regimen (either Days 1, 8, and 30 or Months 0, 1, and 6) to healthy adults aged 65 to 85 years, as measured by C. difficile toxin A- and toxin B-specific neutralizing antibody levels at multiple time points following vaccination; the kinetics of the immune response in healthy adults aged 65 to 85 years for at least up to 12 months following the administration of 3 doses of C. difficile vaccine; the immunogenicity of a fourth dose of C. difficile vaccine as measured by C. difficile toxin A- and toxin B-specific neutralizing antibody levels at multiple time points following vaccination; and the kinetics of the immune response in healthy adults aged 65 to 85 years for at least up to 36 months following the administration of a fourth dose of a C. difficile vaccine.

Exemplary compositions are provided. For instance, compositions comprising an effective amount of C. difficile toxoid A and toxoid B (e.g., from about 40 to about 500 μg/dose, such as about any of 40, 50, 60, 70, 80, 90, 100, 1 10, 120, 130, 140, 1 50, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 μg/dose, such as about 50 to about 100 μg/dose (w/w, total amount of toxoids A and B in the composition)) at an effective toxoid A:B ratio (e.g., about any of 10%, 20%, 30%, 40%, 50%, 60%, 70%, 3: 1 , 3 :2, or 1 : 1 toxoid A to toxoid B by weight), and with a sufficient purity (e.g., at least about 80 to about 100%, such as about any of 80, 85, 90, 95 or 90-100% (w/w)), using one or more administrations (e.g., at least two, three administrations or doses) by any suitable route (e.g., intramuscularly), each dose of a multiple dose administration regimen being suitably separated from one another (e.g., by at least about one to about ten days such as about any of one, two, three, four, five, six, seven, eight, nine or ten, such as about seven days) are provided. The length of time (time interval) between doses would be understood by those of ordinary skill to vary depending on the individual and that that interval should be long enough (e.g., as measured in days) such that the immune response from the prior dose both has time to develop (e.g., to be primed) and is not in any way inhibited by the subsequent dose (e.g., the boosting dose or doses).

In one embodiment, the composition used in the vaccination regimen of the present invention includes from about 40 to about 500 μg/dose of C. difficile toxoid A. In an embodiment the composition includes from about 50 to about 400 μg/dose of C. difficile toxoid A. In one embodiment, the composition includes from about 50 to about 200 μg/dose of C. difficile toxoid A. In one embodiment the composition includes from about 50 to about 150 μg/dose. In one embodiment the composition includes about any of 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 μg/dose of C. difficile toxoid A. In one embodiment, the composition includes about 50 μg/dose of C. difficile toxoid A. In another embodiment, the composition includes about 100 μg/dose of C. difficile toxoid A.

In one embodiment the composition used in the vaccination regimen of the present invention includes from about 40 to about 500 μg/dose of C. difficile toxoid B. In one embodiment the composition includes from about 50 to about 400 μg/dose of C. difficile toxoid B. In one embodiment the composition includes from about 50 to about 200 μg/dose of C. difficile toxoid B. In one embodiment the composition includes from about 50 to about 150 μg/dose. In one embodiment the composition includes about any of 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 μg/dose of C. difficile toxoid B. In one embodiment, the composition includes about 50 μg/dose of C. difficile toxoid B. In another embodiment, the composition includes about 100 μg/dose of C. difficile toxoid B.

In one embodiment the composition used in the vaccination regimen of the present invention includes C. difficile toxoid A and B at the doses disclosed herein. In one embodiment, the toxoid A to B ratio is 3:1, 3:2, or 1:1 toxoid A to toxoid B by weight. In one embodiment, the toxoid A to B ratio is 1:3, 2:3, or 1:1 toxoid A to toxoid B by weight. In one embodiment, the toxoid A to B ratio is 1:1 toxoid A to toxoid B by weight.

In one embodiment the composition used in the vaccination regimen of the present invention includes C. difficile toxoid A and B with a purity of at least about 80 to about 100%. In one embodiment the composition used in the vaccination regimen of the present invention includes C. difficile toxoid A and B with a purity of at least about 90 to about 100%. In one embodiment the composition used in the vaccination regimen of the present invention includes C. difficile toxoid A and B with a purity of about 80, 85, 90, 95 or 100% (w/w).

In one embodiment the compositions disclosed herein are administered once. In one embodiment the compositions disclosed herein are administered two times. In one embodiment the compositions disclosed herein are administered three times. In one embodiment the compositions disclosed herein are administered four times.

In one embodiment the compositions disclosed herein are administered two times at the same dose. In one embodiment the compositions disclosed herein are administered three times at the same dose. In one embodiment the compositions disclosed herein are administered four times at the same dose.

In one embodiment the composition of the present invention are administered by any suitable route. In one embodiment, the compositions disclosed herein are administered by intramuscular, intraperitoneal, intradermal or subcutaneous routes. In one embodiment the compositions disclosed herein are administered subcutaneously or intramuscularly. In one embodiment the compositions disclosed herein are administered intramuscularly.

In one embodiment of the present invention, each dose of a multiple dose administration regimen is suitably separated from one another. In one embodiment, the compositions disclosed herein are administered two times each dose being separated from one another by about one to about ten days. In one embodiment, the compositions disclosed herein are administered two times each dose being separated from one another by about two to nine days. In one embodiment, the compositions disclosed herein are administered two times each dose being separated from one another by about one, two, three, four, five, six, seven, eight, nine or ten days. In one embodiment, the compositions disclosed herein are administered two times each dose being separated from one another by about six days. In one embodiment, the compositions disclosed herein are administered two times each dose being separated from one another by about seven days. In one embodiment, the compositions disclosed herein are administered two times each dose being separated from one another by about eight days. In one embodiment, the compositions disclosed herein are administered two times each dose being separated from one another by about one to about four months. In one embodiment, the compositions disclosed herein are administered two times each dose being separated from one another by about one, two, three or four months. In one embodiment, the compositions disclosed herein are administered two times each dose being separated from one another by about one month. In one embodiment, the compositions disclosed herein are administered two times each dose being separated from one another by about two months.

In one embodiment, the compositions disclosed herein are administered three times the first and second dose being separated from one another by about one to about ten days and the third dose being separated from the first dose by about 15 to 45 days. In one embodiment, the compositions disclosed herein are administered three times the first and second dose being separated from one another by about 5 to about 8 days and the third dose being separated from the first dose by about 20 to 35 days. In one embodiment, the compositions disclosed herein are administered three times the first and second dose being separated from one another by about 6 to about 7 days and the third dose being separated from the first dose by about 25 to 35 days. In one embodiment, the compositions disclosed herein are administered three times the first and second dose being separated from one another by about seven days and the third dose being separated from the first dose by about 30 days.

In one embodiment, the compositions disclosed herein are administered three times the first and second dose being separated from one another by about one to about four months and the third dose being separated from the first dose by about 5 to 10 months. In one embodiment, the compositions disclosed herein are administered three times the first and second dose being separated from one another by about one to two months and the third dose being separated from the first dose by about 5 to 8 months. In one embodiment, the compositions disclosed herein are administered three times the first and second dose being separated from one another by about one month and the third dose being separated from the first dose by about 6 months. In one embodiment, the compositions disclosed herein are administered three times the first and second dose being separated from one another by about one month and the third dose being separated from the first dose by about 5 months. In one embodiment, the compositions disclosed herein are administered three times the first and second dose being separated from one another by about one month and the third dose being separated from the first dose by about 7 months.

In one embodiment, the compositions disclosed herein are administered four times the first and second dose being separated from one another by about one to about ten days, the third dose being separated from the first dose by about 15 to 45 days and the fourth and third dose being separated from one another by about 6 months to about 2 years. In one embodiment, the compositions disclosed herein are administered four times the first and second dose being separated from one another by about 5 to about 8 days, the third dose being separated from the first dose by about 20 to 35 days and the fourth and third dose being separated from one another by about 10 months to about 1.5 years. In one embodiment, the compositions disclosed herein are administered four times the first and second dose being separated from one another by about 6 to about 7 days, the third dose being separated from the first dose by about 25 to 35 days and the fourth and third dose being separated from one another by about 11 months to about 13 months. In one embodiment, the compositions disclosed herein are administered four times the first and second dose being separated from one another by about seven days, the third dose being separated from the first dose by about 30 days and the fourth and third dose being separated from one another by 1 year.

In one embodiment, the compositions disclosed herein are administered four times the first and second dose being separated from one another by about one to about four months, the third dose being separated from the first dose by about 5 to 10 months and the fourth dose being separated from the third dose by about 6 months to 2 years. In one embodiment, the compositions disclosed herein are administered four times the first and second dose being separated from one another by about one to two months, the third dose being separated from the first dose by about 5 to 8 months and the fourth dose being separated from the third dose by about 10 months to 1.5 years. In one embodiment, the compositions disclosed herein are administered four times the first and second dose being separated from one another by about one month, the third dose being separated from the first dose by about 6 months and the fourth dose being separated from the third dose by about 11 months to 13 months. In one embodiment, the compositions disclosed herein are administered four times the first and second dose being separated from one another by about one month, the third dose being separated from the first dose by about 6 months and the fourth dose being separated from the third dose by about 12 months.

In one embodiment the compositions given in any of the multi-dose regimen disclosed herein are given at the same dose (i.e. same quantity of C. difficile toxoid A and/or B). In one embodiment the compositions given in any of the multi-dose regimen disclosed herein are given at the same (i.e. same dose and same ingredients).

In one embodiment the compositions given in any of the multi-dose regimen disclosed herein are given at different doses. In one embodiment the compositions given in any of the multi-dose regimen disclosed herein are given at the same dose of antigen (i.e. same quantity of C. difficile toxoid A and/or B) but may comprise different ingredients (e.g. different adjuvants).

In some embodiments, the second administration is at least one, two, three, four, five, six, seven, eight, nine or ten days after the first administration (e.g., day 0) and the third administration is at least about 20-200 (e.g., about 20, 30, 40, 50, 60, 70, 80, 90, 100, 110, 120, 130, 140, 150, 160, 170, 180, 190, or 200, such as about 30 or about 180 days) days after the first administration. For instance, the method may comprise first, second and/or third administrations wherein the second administration is at least 7 days after the first administration and the third administration is at least about 30 days and/or at least about 180 days after the first or second administration. In some embodiments, the second administration is about seven days after the first administration and the third administration is about 30 days after the first administration.

Upon administration of such compositions using such methods to a host/subject, an immune response is typically observed, which typically includes a humoral immune response and may involve a cellular immune response.

In certain embodiments, the method may comprise administering the immunogenic composition to a human, subject at risk for infection. In some embodiments, the human subject may be at least about any of 40, 50, 65 years or older. In some embodiments, the human subject may be about 40 to about 65 years of age. In some embodiments, the human subject may be 65-75 years of age. Thus, methods for administering the compositions are also provided. Methods for making the compositions are described herein and are available to those of ordinary skill in the art. In one aspect, the invention relates to methods for immunizing a subject (e.g., a human being) against C. difficile by administering thereto a composition comprising one or more antigens of C. difficile. In one aspect, the invention relates to a composition disclosed herein for use in a method for immunizing a subject against C. difficile. In one aspect, the invention relates to a composition disclosed herein for use in a method for immunizing a human subject against C. difficile. In one embodiment, the human subject is 40-90 years of age. In one embodiment, the human subject is 50-85 years of age. In one embodiment, the human subject is 60-85 years of age. In an embodiment, the human subject is 65-85 years of age. In one embodiment, the human subject is 65-69 years of age. In an embodiment, the human subject is 70-79 years of age. In one embodiment, the human subject is 75-79 years of age. In one embodiment, the human subject is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90 years of age. In one embodiment, the human subject is at least 65, 66, 67, 68, 69, 70, 71, 72, 73, 74, 75, 76, 77, 78, 79, 80, 81, 82, 83, 84 or 85 years of age.

For instance, a suitable composition may comprise a total of about 50 or about 100 μg (or about 50-100 μg) C. difficile toxoid (toxoid A and toxoid B) at an approximate toxoid A to toxoid B ratio of about 3:2, with or without adjuvant (e.g., aluminum hydroxide). For comparison purposes, the antigen-containing composition may be administered to one group of subjects and a placebo composition (e.g., 0.9% normal saline) administered (e.g., on the same schedule) to another group. Immunological data and safety data may be obtained from the subjects on particular days (e.g., days 0, 14, 30, 60, 180, and/or 210, and/or up to 1000 days after the first administration). Administration of the composition may take place on, for example, days 0 (first administration), about day 7 (second administration), about day 30 (third administration) and/or about day 180 (alternative third administration or fourth administration).

The composition may comprise C. difficile toxoid A and toxoid B at an effective toxoid A:B ratio (e.g., about any of 3:1 , 3:2, or 1:1 toxoid A to toxoid B by weight) at a sufficient purity (e.g., about 90% or higher purity (w/w)). For instance, the composition may comprise a highly purified (e.g., >90% (w/w/)) preparation of C. difficile toxoids A & B in an approximate toxoid A to toxoid B ratio of about 3:2. Such compositions may be prepared using any of the available methods of preparation, e.g., as described in WIPO Patent Application WO/2012/143902, U.S. Pat. No. 9,187,536, and WIPO Patent Application WO/2014/060898, which are each incorporated by reference herein in their respective entireties.

The term “C. difficile toxoid” is used herein to refer to a C. difficile toxin (Toxin A or Toxin B) that has been partially or completely inactivated. A toxin is inactivated if it has less toxicity (e.g., 100%, 99%, 98%, 80%, 70%, 60%, 50%, 40%, 30%, 20%, 10% or less toxicity or any value therebetween) than untreated toxin, as measured by for example an in vitro cytotoxicity assay or by animal toxicity. C. difficile toxoids can be produced by purification of toxins from C. difficile cultures and inactivation of toxins by chemical (e.g., formaldehyde, glutaraldehyde, peroxide or oxygen treatment). Alternatively, wild type or mutant C. difficile toxins that lack or have reduced toxicity can be produced using recombinant methods and/or alternative chemical crosslinking agents. For example, genetic mutations resulting in reduced toxicity can be made. Wild type or mutant C. difficile toxins lacking specific regions to reduce toxicity can also be made.

The C. difficile toxoid or mutant C. difficile toxin refers to a molecule that exhibits a structure or sequence that differs from the corresponding wild-type structure or sequence, e.g., by having crosslinks as compared to the corresponding wild-type structure and/or by having at least one mutation, as compared to the corresponding wild-type sequence when optimally aligned, such as by the programs GAP or BESTFIT using default gap weights. The term toxoid or mutant toxin as used herein further exhibits a functional property (e.g., abrogated glucosyltransferase and/or abrogated cysteine protease activity) that differs from the corresponding wild-type molecule.

The toxoid as used herein may be any of the toxoids or mutant C. difficile toxins as described in WIPO Patent Application WO/2012/143902, U.S. Pat. No. 9187536, and WIPO Patent Application WO/2014/060898, which are each incorporated by reference herein in their respective entireties. That is, the toxoid as used herein may be any of the polypeptides as described in WIPO Patent Application WO/2012/143902, U.S. Pat. No. 9187536, and WIPO Patent Application WO/2014/060898, which are each incorporated by reference herein in their respective entireties. A C. difficile toxin from any of the wild-type strains described above may be used as a source from which a toxoid or mutant C. difficile toxin is produced. Preferably, C. difficile 630 is the source from which a C. difficile toxoid is produced.

In one embodiment, the toxoid refers to a polypeptide that has any one sequence selected from SEQ ID NO: 1 to SEQ ID NO: 761, wherein the initial methionine is absent, and wherein the polypeptide has been contacted with a chemical crosslinker, such as, for example, formaldehyde or EDC, as described herein, and/or has been genetically mutated. More specifically, in one embodiment, the toxoid is a polypeptide having the amino acid sequence set forth in any one of SEQ ID NOs: 1-8, 15, 17, 19, 21, 23, 25, 28-35, 82-761.

The mutation may involve a substitution, deletion, truncation or modification of the wild type amino acid residue normally located at that position. Preferably, the mutation is a non-conservative amino acid substitution. The mutant toxins of the invention may be prepared by techniques known in the art for preparing mutations, such as, for example, site-directed mutagenesis, mutagenesis using a mutagen (e.g., UV light), etc. Preferably, site-directed mutagenesis is used. Alternatively, a nucleic acid molecule having an objective sequence may be directly synthesized. Such chemical synthesis methods are known in the art.

In the present invention, the mutant C. difficile toxin includes at least one mutation in a glucosyltransferase domain, relative to the corresponding wild-type C. difficile toxin. In one embodiment, the glucosyltransferase domain includes at least two mutations. Preferably, the mutation decreases or abrogates glucosyltransferase enzyme activity of the toxin, as compared to the glucosyltransferase enzyme activity of the corresponding wild-type C. difficile toxin.

An exemplary C. difficile toxoid A includes a glucosyltransferase domain including SEQ ID NO: 29 having an amino acid substitution at positions 285 and 287, and a cysteine protease domain comprising SEQ ID NO: 32 having an amino acid substitution at position 158, relative to the corresponding wild-type C. difficile toxin A. For example, such a mutant C. difficile TcdA includes the amino acid sequence set forth in SEQ ID NO: 4, wherein the initial methionine is not present. In another embodiment, the mutant C. difficile toxin A includes the amino acid sequence set forth in SEQ ID NO: 84. Further examples of a C. difficile toxoid A include the amino acid sequence set forth in SEQ ID NO: 7, which has a D269A, R272A, D285A, D287A, E460A, R462A, and C700A mutation, as compared to SEQ ID NO: 1, wherein the initial methionine is optionally not present. In another embodiment, the mutant C. difficile toxin A includes the amino acid sequence set forth in SEQ ID NO: 83.

An exemplary C. difficile toxoid B includes the amino acid sequence set forth in SEQ ID NO: 6, wherein the initial methionine is not present. In another embodiment, the mutant C. difficile toxin A includes the amino acid sequence set forth in SEQ ID NO: 86. Further examples of a mutant C. difficile TcdB include the amino acid sequence set forth in SEQ ID NO: 8, which has a D270A, R273A, D286A, D288A, D461A, K463A, and C698A mutation, as compared to SEQ ID NO: 2, and wherein the initial methionine of SEQ ID NO: 8 is optionally not present. In another embodiment, the mutant C. difficile toxin B includes the amino acid sequence set forth in SEQ ID NO: 85.

In addition to generating an immune response in a mammal, the toxoids described herein also have reduced cytotoxicity compared to the corresponding wild-type C. difficile toxin. Preferably, the immunogenic compositions are safe and have minimal (e.g., about a 6-8 logo reduction) to no cytotoxicity, relative to the cytotoxicity of a respective wild-type toxin, for administration in mammals.

As used herein, the term cytotoxicity is a term understood in the art and refers to apoptotic cell death and/or a state in which one or more usual biochemical or biological functions of a cell are aberrantly compromised, as compared to an identical cell under identical conditions but in the absence of the cytotoxic agent. Toxicity can be quantitated, for example, in cells or in mammals as the amount of an agent needed to induce 50% cell death (i.e., EC₅₀ or ED₅₀, respectively) or by other methods known in the art.

Assays for indicating cytotoxicity are known in the art, such as cell rounding assays. Additional exemplary cytotoxicity assays known in the art include glucosylation assays relating to phosphorimaging of Ras labeled with [¹⁴C]glucose assays and preferably the in vitro cytotoxicity assay described in WIPO Patent Application WO/2012/143902, U.S. Pat. No. 9,187,536, and WIPO Patent Application WO/2014/060898, which are each incorporated by reference herein in their respective entireties, wherein EC₅₀ may refer to a concentration of an immunogenic composition that exhibits at least about 50% of cytopathogenic effect (CPE) in a cell, preferably a human diploid fibroblast cell (e.g., IMR90 cell (ATCC CCL-186™), as compared to an identical cell under identical conditions in the absence of the toxin. The in vitro cytotoxicity assay may also be used to assess the concentration of a composition that inhibits at least about 50% of a wild-type C. difficile toxin-induced cytopathogenic effect (CPE) in a cell, preferably a human diploid fibroblast cell (e.g., IMR90 cell (ATCC CCL-186™), as compared to an identical cell under identical conditions in the absence of the toxin.

In one embodiment, the cytotoxicity of the immunogenic composition is reduced by at least about 1000, 2000, 3000, 4000, 5000-, 6000-, 7000-, 8000-, 9000-, 10000-, 11000-, 12000-, 13000-fold, 14000-fold, 15000-fold, or more, as compared to the corresponding wild-type C. difficile toxin.

In another embodiment, the cytotoxicity of the immunogenic composition is reduced by at least about 2-log₁₀, more preferably by about 3-log₁₀, and most preferably by about 4-log₁₀ or more, relative to the corresponding wild-type toxin under identical conditions. For example, a mutant C. difficile TcdB may have an EC₅₀ value of about 10⁻⁹ g/ml as measured in a standard cytopathic effect assay (CPE), as compared to an exemplary wild-type C. difficile TcdB which may have an EC₅₀ value of at least about 10⁻¹² g/ml.

In yet another embodiment, the cytotoxicity of the mutant C. difficile toxin has an EC₅₀ of at least about 50 μg/ml, 100 μg/ml, 200 μg/ml, 300 μg/ml, 400 μg/ml, 500 μg/ml, 600 μg/ml, 700 μg/ml, 800 μg/ml, 900 μg/ml, 1000 μg/ml or greater, as measured by, for example, an in vitro cytotoxicity assay. Accordingly, in a preferred embodiment, the immunogenic compositions and mutant toxins are biologically safe for administration to mammals.

In one embodiment, the toxoid is a polypeptide that has any one sequence selected from SEQ ID NO: 1 to SEQ ID NO: 761, more specifically, the toxoid is a polypeptide having the amino acid sequence set forth in any one of SEQ ID NOs: 1-8, 15, 17, 19, 21, 23, 25, 28-35, 82-761, wherein the initial methionine is absent, and wherein the polypeptide has been contacted with a chemical crosslinker, such as, for example, formaldehyde or EDC, as described herein. Crosslinking (also referred to as “chemical inactivation” or “inactivation” herein) is a process of chemically joining two or more molecules by a covalent bond. The terms “crosslinking reagents,” “crosslinking agents,” and “crosslinkers” refer to molecules that are capable of reacting with and/or chemically attaching to specific functional groups (primary amines, sulfhydryls, carboxyls, carbonyls, etc.) on peptides, polypeptides, and/or proteins. In one embodiment, the molecule may contain two or more reactive ends that are capable of reacting with and/or chemically attaching to specific functional groups (primary amines, sulfhydryls, carboxyls, carbonyls, etc.) on peptides, polypeptides, and/or proteins. Preferably, the chemical crosslinking agent is water-soluble. In another preferred embodiment, the chemical crosslinking agent is a heterobifunctional crosslinker. In another embodiment, the chemical crosslinking agent is not a bifunctional crosslinker. Chemical crosslinking agents are known in the art.

Exemplary suitable chemical crosslinking agents include formaldehyde; formalin; acetaldehyde; propionaldehyde; water-soluble carbodiimides (RN═C═NR′), which include 1-Ethyl-3-(3-Dimethylaminopropyl)-Carbodiimide (EDC), 1-Ethyl-3-(3-Dimethylaminopropyl)-Carbodiimide Hydrochloride, 1-Cyclohexyl-3-(2-morpholinyl-(4-ethyl)carbodiimide metho-p-toluenesulfonate (CMC), N,N′-dicyclohexylcarbodiimide (DCC), and N,N′-diisopropylcarbodiimide (DIC), and derivatives thereof; and N-hydroxysuccinimide (NHS); phenylglyoxal; and/or UDP-dialdehyde.

Preferably, the crosslinking agent is EDC. When a mutant C. difficile toxin polypeptide is chemically modified by EDC (e.g., by contacting the polypeptide with EDC), in one embodiment, the polypeptide includes (a) at least one crosslink between a side chain of an aspartic acid residue of the polypeptide and a side chain of a lysine residue of the polypeptide. In one embodiment, the polypeptide includes (b) at least one crosslink between a side chain of a glutamic acid residue of the polypeptide and a side chain of a lysine residue of the polypeptide. In one embodiment, the polypeptide includes (c) at least one crosslink between the carboxyl group at the C-terminus of the polypeptide and the amino group of the N-terminus of the polypeptide. In one embodiment, the polypeptide includes (d) at least one crosslink between the carboxyl group at the C-terminus of the polypeptide and a side chain of a lysine residue of the polypeptide. In one embodiment, the polypeptide includes (e) at least one crosslink between a side chain of an aspartic acid residue of the polypeptide and a side chain of a lysine residue of a second isolated polypeptide. In one embodiment, the polypeptide includes (f) at least one crosslink between a side chain of a glutamic acid residue of the polypeptide and a side chain of a lysine residue of a second isolated polypeptide. In one embodiment, the polypeptide includes (g) at least one crosslink between the carboxyl group at the C-terminus of the polypeptide and the amino group of the N-terminus of a second isolated polypeptide. In one embodiment, the polypeptide includes (h) at least one crosslink between the carboxyl group at the C-terminus of the polypeptide and a side chain of a lysine residue of a second isolated polypeptide.

The “second isolated polypeptide” refers to any isolated polypeptide that is present during the reaction with EDC. In one embodiment, the second isolated polypeptide is a mutant C. difficile toxin polypeptide having an identical sequence as the first isolated polypeptide. In another embodiment, the second isolated polypeptide is a mutant C. difficile toxin polypeptide having a different sequence from the first isolated polypeptide.

In one embodiment, the polypeptide includes at least two modifications selected from the (a)-(d) modifications. In an exemplary embodiment, the polypeptide includes (a) at least one crosslink between a side chain of an aspartic acid residue of the polypeptide and a side chain of a lysine residue of the polypeptide and (b) at least one crosslink between a side chain of a glutamic acid residue of the polypeptide and a side chain of a lysine residue of the polypeptide. In a further embodiment, the polypeptide includes at least three modifications selected from the (a)-(d) modifications. In yet a further embodiment, the polypeptide includes the (a), (b), (c), and (d) modifications.

When more than one mutant polypeptide is present during chemical modification by EDC, in one embodiment, the resulting composition includes at least one of any of the (a)-(h) modifications. In one embodiment, the composition includes at least two modifications selected from the (a)-(h) modifications. In a further embodiment, the composition includes at least three modifications selected from the (a)-(h) modifications. In yet a further embodiment, the composition includes at least four modifications selected from the (a)-(h) modifications. In another embodiment, the composition includes at least one of each of the (a)-(h) modifications.

In an exemplary embodiment, the resulting composition includes (a) at least one crosslink between a side chain of an aspartic acid residue of the polypeptide and a side chain of a lysine residue of the polypeptide; and (b) at least one crosslink between a side chain of a glutamic acid residue of the polypeptide and a side chain of a lysine residue of the polypeptide. In one embodiment, the composition further includes (c) at least one crosslink between the carboxyl group at the C-terminus of the polypeptide and the amino group of the N-terminus of the polypeptide; and (d) at least one crosslink between the carboxyl group at the C-terminus of the polypeptide and a side chain of a lysine residue of the polypeptide.

In another exemplary embodiment, the resulting composition includes (e) at least one crosslink between a side chain of an aspartic acid residue of the polypeptide and a side chain of a lysine residue of a second isolated polypeptide; (f) at least one crosslink between a side chain of a glutamic acid residue of the polypeptide and a side chain of a lysine residue of a second isolated polypeptide; (g) at least one crosslink between the carboxyl group at the C-terminus of the polypeptide and the amino group of the N-terminus of a second isolated polypeptide; and (h) at least one crosslink between the carboxyl group at the C-terminus of the polypeptide and a side chain of a lysine residue of a second isolated polypeptide.

In a further exemplary embodiment, the resulting composition includes (a) at least one crosslink between a side chain of an aspartic acid residue of the polypeptide and a side chain of a lysine residue of the polypeptide; (b) at least one crosslink between a side chain of a glutamic acid residue of the polypeptide and a side chain of a lysine residue of the polypeptide; (e) at least one crosslink between a side chain of an aspartic acid residue of the polypeptide and a side chain of a lysine residue of a second isolated polypeptide; and (f) at least one crosslink between a side chain of a glutamic acid residue of the polypeptide and a side chain of a lysine residue of a second isolated polypeptide.

In a preferred embodiment, the chemical crosslinking agent includes formaldehyde, more preferably, an agent including formaldehyde in the absence of lysine. Glycine or other appropriate compound with a primary amine can be used as the quencher in crosslinking reactions. Accordingly, in another preferred embodiment, the chemical agent includes formaldehyde and use of glycine.

In yet another preferred embodiment, the chemical crosslinking agent includes EDC and NHS. As is known in the art, NHS may be included in EDC coupling protocols. However, the inventors surprisingly discovered that NHS may facilitate in further decreasing cytotoxicity of the mutant C. difficile toxin, as compared to the corresponding wild-type toxin, as compared to a genetically mutated toxin, and as compared to a genetically mutated toxin that has been chemically crosslinked by EDC. See, for example, Example 22 described in WIPO Patent Application WO/2012/143902, U.S. Pat. No. 9,187,536, and WIPO Patent Application WO/2014/060898, which are each incorporated by reference herein in their respective entireties. Accordingly, without being bound by mechanism or theory, a mutant toxin polypeptide having a beta-alanine moiety linked to a side chain of at least one lysine residue of the polypeptide (e.g., resulting from a reaction of the mutant toxin polypeptide, EDC, and NHS) may facilitate in further decreasing cytotoxicity of the mutant toxin, as compared to, for example, a C. difficile toxin (wild-type or mutant) wherein a beta-alanine moiety is absent.

Use of EDC and/or NHS may also include use of glycine or other appropriate compound with a primary amine as the quencher. Any compound having a primary amine may be used as a quencher, such as, for example glycine methyl ester and alanine. In a preferred embodiment, the quencher compound is a non-polymeric hydrophilic primary amine. Examples of a non-polymeric hydrophilic primary amine include, for example, amino sugars, amino alcohols, and amino polyols. Specific examples of a non-polymeric hydrophilic primary amine include glycine, ethanolamine, glucamine, amine functionalized polyethylene glycol, and amine functionalized ethylene glycol oligomers. In one embodiment, the chemical crosslinking agent does not include formaldehyde. In one embodiment, the chemical crosslinking agent does not include formalin.

In one aspect, the invention relates to a mutant C. difficile toxin, i.e., a polypeptide, having at least one amino acid side chain chemically modified by EDC and a non-polymeric hydrophilic primary amine, preferably glycine. The resulting glycine adducts (e.g., from a reaction of triple mutant toxins treated with EDC, NHS, and quenched with glycine) may facilitate in decreasing cytotoxicity of the mutant toxin as compared to the corresponding wild-type toxin.

In one embodiment, when a mutant C. difficile toxin, i.e., a polypeptide, is chemically modified by EDC and glycine, the polypeptide includes at least one modification when the polypeptide is modified by EDC (e.g., at least one of any of the (a)-(h) modifications described above), and at least one of the following exemplary modifications: (i) a glycine moiety linked to the carboxyl group at the C-terminus of the polypeptide; (j) a glycine moiety linked to a side chain of at least one aspartic acid residue of the polypeptide; and (k) a glycine moiety linked to a side chain of at least one glutamic acid residue of the polypeptide.

In one embodiment, at least one amino acid of the mutant C. difficile TcdA, i.e., the polypeptide, is chemically crosslinked and/or at least one amino acid of the mutant C. difficile TcdB, i.e., a polypeptide, is chemically crosslinked. Any of the mutant toxins, i.e., polypeptides, described herein may be chemically crosslinked. In another embodiment, at least one amino acid of the polypeptide having SEQ ID NO: 4, SEQ ID NO: 6, SEQ ID NO: 7, and/or SEQ ID NO: 8 is chemically crosslinked. In one embodiment, at least one amino acid residue of a polypeptide having the amino acid sequence of any of SEQ ID NOs: 1 through SEQ ID NO: 761 is crosslinked. For example, in one embodiment, at least one amino acid residue of a polypeptide having the amino acid sequence set forth in any one of SEQ ID NOs: 1-8, 15, 17, 19, 21, 23, 25, 28-35, 82-761, is crosslinked. In another embodiment, at least one amino acid residue of a polypeptide having the amino acid sequence of any of SEQ ID NOs: 183 through SEQ ID NO: 761 includes a modification as described above, e.g., any of the (a)-(k) modifications, such as (a) at least one crosslink between a side chain of an aspartic acid residue of the polypeptide and a side chain of a lysine residue of the polypeptide.

For example, the at least one amino acid may be chemically crosslinked by an agent that includes a carbodiimide, such as EDC. Carbodiimides may form a covalent bond between free carboxyl (e.g., from the side chains of aspartic acid and/or glutamic acid) and amino groups (e.g., in the side chain of lysine residues) to form stable amide bonds.

As another example, the at least one amino acid may be chemically crosslinked by an agent that includes NHS. NHS ester-activated crosslinkers may react with primary amines (e.g., at the N-terminus of each polypeptide chain and/or in the side chain of lysine residues) to yield an amide bond.

In another embodiment, the at least one amino acid may be chemically crosslinked by an agent that includes EDC and NHS. For example, in one embodiment, the invention relates to an isolated polypeptide having the amino acid sequence set forth in SEQ ID NO: 4, wherein the methionine residue at position 1 is optionally not present, wherein the polypeptide includes at least one amino acid side chain chemically modified by EDC and NHS. In another embodiment, the invention relates to an isolated polypeptide having the amino acid sequence set forth in SEQ ID NO: 6, wherein the methionine residue at position 1 is optionally not present, wherein the polypeptide includes at least one amino acid side chain chemically modified by EDC and NHS. In yet another embodiment, the invention relates to an isolated polypeptide having the amino acid sequence set forth in SEQ ID NO: 84, SEQ ID NO: 86, SEQ ID NO: 83, SEQ ID NO: 85, SEQ ID NO: 7, or SEQ ID NO: 8. The polypeptide is modified by contacting the polypeptide with EDC and NHS.

When a mutant C. difficile toxin, i.e., a polypeptide, is chemically modified by (e.g., by contacting) EDC and NHS, in one embodiment, the polypeptide includes at least one modification when the polypeptide is modified by EDC (e.g., at least one of any of the (a)-(h) modifications described above), and (l) a beta-alanine moiety linked to a side chain of at least one lysine residue of the polypeptide.

In another aspect, the invention relates to a mutant C. difficile toxin, i.e., a polypeptide, wherein the polypeptide includes at least one amino acid side chain chemically modified by EDC, NHS, and a non-polymeric hydrophilic primary amine, preferably glycine. In one embodiment, the polypeptide includes at least one modification when the polypeptide is modified by EDC (e.g., at least one of any of the (a)-(h) modifications described above), at least one modification when the polypeptide is modified by glycine (e.g., at least one of any of the (i)-(k) modifications described above), and (l) a beta-alanine moiety linked to a side chain of at least one lysine residue of the polypeptide.

In one aspect, the invention relates to a mutant C. difficile toxin, i.e., a polypeptide, wherein a side chain of at least one lysine residue of the polypeptide is linked to a beta-alanine moiety. In one embodiment, a side chain of a second lysine residue of the polypeptide is linked to a side chain of an aspartic acid residue and/or to a side chain of a glutamic acid residue. The “second” lysine residue of the polypeptide includes a lysine residue of the polypeptide that is not linked to a beta-alanine moiety. The side chain of an aspartic acid and/or the side chain of a glutamic acid to which the second lysine residue is linked may be that of the polypeptide to form an intra-molecular crosslink, or that of a second polypeptide to form an inter-molecular crosslink. In another embodiment, a side chain of at least one aspartic acid residue and/or a side chain of at least one glutamic acid residue of the polypeptide is linked to a glycine moiety. The aspartic acid residue and/or the glutamic acid residue that is linked to a glycine moiety is not also linked to a lysine residue.

In another aspect, the invention relates to a mutant C. difficile toxin, i.e., a polypeptide, wherein at least one amino acid side chain of a wild-type C. difficile toxin is chemically modified. In one embodiment, at least one amino acid side chain of a wild-type C. difficile toxin A and/or at least one amino acid side chain of a wild-type C. difficile toxin B is chemically modified by EDC. For example, in one embodiment, TcdA (SEQ ID NO: 1) and/or Tcdb (SEQ ID NO: 2) is chemically modified by EDC. In another embodiment, the wild-type toxin is chemically modified by EDC and NHS. In one embodiment, the mutant toxin, i.e., polypeptide, includes a chemically modified wild-type toxin A, wherein the wild-type toxin A is any one described in Table 1. In another embodiment, the mutant toxin, i.e., polypeptide, includes a chemically modified wild-type toxin B, wherein the wild-type toxin B is any one described in Table 2.

TABLE 1
Wild-type C. difficile Strains

| C. difficile Strain ID | Toxin A, SEQ ID NO: |
|---|---|
| 2004013 | SEQ ID NO: 87 |
| 2004111 | SEQ ID NO: 88 |
| 2004118 | SEQ ID NO: 89 |
| 2004205 | SEQ ID NO: 90 |
| 2004206 | SEQ ID NO: 91 |
| 2005022 | SEQ ID NO: 92 |
| 2005088 | SEQ ID NO: 93 |
| 2005283 | SEQ ID NO: 94 |
| 2005325 | SEQ ID NO: 95 |
| 2005359 | SEQ ID NO: 96 |
| 2006017 | SEQ ID NO: 97 |
| 2006376 | N/A |
| 2007070 | SEQ ID NO: 98 |
| 2007217 | SEQ ID NO: 99 |
| 2007302 | SEQ ID NO: 100 |
| 2007816 | SEQ ID NO: 101 |
| 2007838 | SEQ ID NO: 102 |
| 2007858 | SEQ ID NO: 103 |
| 2007886 | SEQ ID NO: 104 |
| 2008222 | SEQ ID NO: 105 |
| 2009078 | SEQ ID NO: 106 |
| 2009087 | SEQ ID NO: 107 |
| 2009141 | SEQ ID NO: 108 |
| 2009292 | SEQ ID NO: 109 |
| 001 | SEQ ID NO: 148 |
| 002 | SEQ ID NO: 149 |
| 003 | SEQ ID NO: 150 |
| 012 (004) | SEQ ID NO: 151 |
| 014 | SEQ ID NO: 134 |
| 015 | SEQ ID NO: 135 |
| 017 | |
| 020 | SEQ ID NO: 136 |
| 023 | SEQ ID NO: 137 |
| 027 | SEQ ID NO: 138 |
| 029 | SEQ ID NO: 139 |
| 046 | SEQ ID NO: 140 |
| 053 | SEQ ID NO: 168 |
| 059 | SEQ ID NO: 178 |
| 070 | SEQ ID NO: 152 |
| 075 | SEQ ID NO: 153 |
| 077 | SEQ ID NO: 154 |
| 078 | SEQ ID NO: 169 |
| 081 | SEQ ID NO: 155 |
| 087 | SEQ ID NO: 170 |
| 095 | SEQ ID NO: 171 |
| 106 | SEQ ID NO: 180 |
| 117 | SEQ ID NO: 156 |
| 126 | SEQ ID NO: 172 |
| 131 | SEQ ID NO: 157 |
| SE844 | SEQ ID NO: 196 |
| 12087 | SEQ ID NO: 197 |
| K14 | SEQ ID NO: 198 |
| BI6 | SEQ ID NO: 199 |
| BI17 | SEQ ID NO: 200 |
| CH6230 | SEQ ID NO: 201 |
| SE881 | SEQ ID NO: 202 |

TABLE 2
Wild-type C. difficile Strains

| C. difficile Strain ID | Toxin B, SEQ ID NO: |
|---|---|
| 2004013 | SEQ ID NO: 110 |
| 2004111 | SEQ ID NO: 111 |
| 2004118 | SEQ ID NO: 112 |
| 2004205 | SEQ ID NO: 113 |
| 2004206 | SEQ ID NO: 114 |
| 2005022 | SEQ ID NO: 115 |
| 2005088 | SEQ ID NO: 116 |
| 2005283 | SEQ ID NO: 117 |
| 2005325 | SEQ ID NO: 118 |
| 2005359 | SEQ ID NO: 119 |
| 2006017 | SEQ ID NO: 120 |
| 2006376 | SEQ ID NO: 121 |
| 2007070 | SEQ ID NO: 122 |
| 2007217 | SEQ ID NO: 123 |
| 2007302 | SEQ ID NO: 124 |
| 2007816 | SEQ ID NO: 125 |
| 2007838 | SEQ ID NO: 126 |
| 2007858 | SEQ ID NO: 127 |
| 2007886 | SEQ ID NO: 128 |
| 2008222 | SEQ ID NO: 129 |
| 2009078 | SEQ ID NO: 130 |
| 2009087 | SEQ ID NO: 131 |
| 2009141 | SEQ ID NO: 132 |
| 2009292 | SEQ ID NO: 133 |
| 001 | SEQ ID NO: 158 |
| 002 | SEQ ID NO: 159 |
| 003 | SEQ ID NO: 160 |
| 012 (004) | SEQ ID NO: 161 |
| 014 | SEQ ID NO: 141 |
| 015 | SEQ ID NO: 142 |
| 017 | SEQ ID NO: 182 |
| 020 | SEQ ID NO: 143 |
| 023 | SEQ ID NO: 144 |
| 027 | SEQ ID NO: 145 |
| 029 | SEQ ID NO: 146 |
| 046 | SEQ ID NO: 147 |
| 053 | SEQ ID NO: 173 |
| 059 | SEQ ID NO: 179 |
| 070 | SEQ ID NO: 162 |
| 075 | SEQ ID NO: 163 |
| 077 | SEQ ID NO: 164 |
| 078 | SEQ ID NO: 174 |
| 081 | SEQ ID NO: 165 |
| 087 | SEQ ID NO: 175 |
| 095 | SEQ ID NO: 176 |
| 106 | SEQ ID NO: 181 |
| 117 | SEQ ID NO: 166 |
| 126 | SEQ ID NO: 177 |
| 131 | SEQ ID NO: 167 |

As yet another example of a chemically crosslinked mutant C. difficile toxin, i.e., a polypeptide, the at least one amino acid may be chemically crosslinked by an agent that includes formaldehyde. Formaldehyde may react with the amino group of an N-terminal amino acid residue and the side-chains of arginine, cysteine, histidine, and lysine. Formaldehyde and glycine may form a Schiff-base adduct, which may attach to primary N-terminal amino groups, arginine, and tyrosine residues, and to a lesser degree asparagine, glutamine, histidine, and tryptophan residues.

A chemical crosslinking agent is said to reduce cytotoxicity of a toxin if the treated toxin has less toxicity (e.g., about 100%, 99%, 95%, 90%, 80%, 75%, 60%, 50%, 25%, or 10% less toxicity) than untreated toxin under identical conditions, as measured, for example, by an in vitro cytotoxicity assay, or by animal toxicity.

Preferably, the chemical crosslinking agent reduces cytotoxicity of the mutant C. difficile toxin by at least about a 2-login reduction, more preferably about a 3-login reduction, and most preferably about a 4-login or more, relative to the mutant toxin under identical conditions but in the absence of the chemical crosslinking agent. As compared to the wild-type toxin, the chemical crosslinking agent preferably reduces cytotoxicity of the mutant toxin by at least about a 5-log₁₀ reduction, about a 6-log₁₀ reduction, about a 7-log₁₀ reduction, about an 8-log₁₀ reduction, or more.

In another preferred embodiment, the chemically inactivated mutant C. difficile toxin, i.e., a polypeptide, exhibits EC₅₀ value of greater than or at least about 50 μg/ml, 100 μg/ml, 200 μg/ml, 300 μg/ml, 400 μg/ml, 500 μg/ml, 600 μg/ml, 700 μg/ml, 800 μg/ml, 900 μg/ml, 1000 μg/ml or greater, as measured by, for example, an in vitro cytotoxicity assay, such as one described herein.

Reaction conditions for contacting the mutant toxin with the chemical crosslinking agent are within the scope of expertise of one skilled in the art, and the conditions may vary depending on the agent used. However, the inventors surprisingly discovered optimal reaction conditions for contacting a mutant C. difficile toxin, i.e., a polypeptide, with a chemical crosslinking agent, while retaining functional epitopes and decreasing cytotoxicity of the mutant toxin, as compared to the corresponding wild-type toxin.

Preferably, the reaction conditions are selected for contacting a mutant toxin with the crosslinking agent, wherein the mutant toxin has a minimum concentration of about 0.5, 0.75, 1.0, 1.25, 1.5, 1.75, 2.0 mg/ml to a maximum of about 3.0, 2.5, 2.0, 1.5, or 1.25 mg/ml. Any minimum value may be combined with any maximum value to define a range of suitable concentrations of a mutant toxin for the reaction. Most preferably, the mutant toxin has a concentration of about 1.0-1.25 mg/ml for the reaction.

In one embodiment, the agent used in the reaction has a minimum concentration of about 1 mM, 2 mM, 3 mM, 4 mM, 5 mM, 10 mM, 15 mM, 20 mM, 30 mM, 40 mM, or 50 mM, and a maximum concentration of about 100 mM, 90 mM, 80 mM, 70 mM, 60 mM, or 50 mM. Any minimum value may be combined with any maximum value to define a range of suitable concentrations of the chemical agent for the reaction.

In a preferred embodiment wherein the agent includes formaldehyde, the concentration used is preferably any concentration between about 2 mM to 80 mM, most preferably about 40 mM. In another preferred embodiment wherein the agent includes EDC, the concentration used is preferably any concentration between about 1.3 mM to about 13 mM, more preferably about 2 mM to 3 mM, most preferably about 2.6 mM. In one embodiment, the concentration of EDC is at most 5 g/L, 4 g/L, 3 g/L, 2.5 g/L, 2 g/L, 1.5 g/L, 1.0 g/L, 0.5 g/L based on the total reaction volume, preferably at most 1 g/L, more preferably at most 0.5 g/L.

Exemplary reaction times in which the mutant toxin is contacted with the chemical crosslinking agent include a minimum of about 0.5, 1, 2, 3, 4, 5, 6, 12, 24, 36, 48, or 60 hours, and a maximum of about 14 days, 12 days, 10 days, 7 days, 5 days, 3 days, 2 days, 1 day, or 12 hours, 11, 10, 9, 8, 7, 6, 5, 4, 3, 2, or 1 hour. Any minimum value may be combined with any maximum value to define a range of suitable reaction times.

In a preferred embodiment, the step of contacting the mutant toxin with the chemical crosslinking agent occurs for a period of time that is sufficient to reduce cytotoxicity of the mutant C. difficile toxin to an EC₅₀ value of at least about 1000 μg/ml in a suitable human cell, e.g., IMR-90 cells, in a standard in vitro cytotoxicity assay, as compared to an identical mutant toxin in the absence of the crosslinking agent. More preferably, the reaction step is carried out for a time that is at least twice as long, and most preferably at least three times as long or more, as the period of time sufficient to reduce the cytotoxicity of the mutant toxin to an EC₅₀ value of at least about 1000 μg/ml in a suitable human cell. In one embodiment, the reaction time does not exceed about 168 hours (or 7 days).

For example, in one embodiment wherein the agent includes formaldehyde, the mutant toxin is preferably contacted with the agent for about 12 hours, which was shown to be an exemplary period of time that was sufficient to reduce cytotoxicity of the mutant C. difficile toxin to an EC₅₀ value of at least about 1000 μg/ml in a suitable human cell, e.g., IMR-90 cells, in a standard in vitro cytotoxicity assay, as compared to an identical mutant toxin in the absence of the crosslinking agent. In a more preferred embodiment, the reaction is carried out for about 48 hours, which is at least about three times as long as a sufficient period of time for the reaction. In such an embodiment, the reaction time is preferably not greater than about 72 hours.

In another embodiment wherein the agent includes EDC, the mutant toxin is preferably contacted with the agent for about 0.5 hours, more preferably at least about 1 hour, or most preferably about 2 hours. In one embodiment, the mutant toxin is contacted with EDC for at most about 5 hours, preferably at most about 3 hours, more preferably at most about 2 hours. In such an embodiment, the reaction time is preferably not greater than about 6 hours.

Exemplary pH at which the mutant toxin is contacted with the chemical crosslinking agent include a minimum of about pH 5.5, 6.0, 6.5, 7.0, or 7.5, and a maximum of about pH 8.5, 8.0, 7.5, 7.0, or 6.5. Any minimum value may be combined with any maximum value to define a range of suitable pH. Preferably, the reaction occurs at pH 6.5 to 7.5, preferably at pH 7.0.

Exemplary temperatures at which the mutant toxin is contacted with the chemical crosslinking agent include a minimum of about 2° C., 4° C., 10° C., 20° C., 25° C., or 37° C., and a maximum temperature of about 40° C., 37° C., 30° C., 27° C., 25° C., or 20° C. Any minimum value may be combined with any maximum value to define a range of suitable reaction temperature. Preferably, the reaction occurs at about 20° C. to 30° C., most preferably at about 25° C.

The immunogenic compositions described above may include one mutant C. difficile toxin (A or B), i.e., polypeptides. Accordingly, the immunogenic compositions can occupy separate vials (e.g., a separate vial for a composition including mutant C. difficile toxin A and a separate vial for a composition including mutant C. difficile toxin B) in the preparation or kit. The immunogenic compositions may be intended for simultaneous, sequential, or separate use.

In another embodiment, the immunogenic compositions described above may include both mutant C. difficile toxins (A and B), i.e., polypeptides. Any combination of mutant C. difficile toxin A and mutant C. difficile toxin B described may be combined for an immunogenic composition. Accordingly, the immunogenic compositions can be combined in a single vial (e.g., a single vial containing both a composition including mutant C. difficile TcdA and a composition including mutant C. difficile TcdB). Preferably, the immunogenic compositions include a mutant C. difficile TcdA and a mutant C. difficile TcdB, i.e., polypeptides.

For example, in one embodiment, the immunogenic composition includes SEQ ID NO: 4 and SEQ ID NO: 6, wherein at least one amino acid of each of SEQ ID NO: 4 and SEQ ID NO: 6 is chemically crosslinked. In another embodiment, the immunogenic composition includes a mutant C. difficile toxin A, which includes SEQ ID NO: 4 or SEQ ID NO: 7, and a mutant C. difficile toxin B, which comprises SEQ ID NO: 6 or SEQ ID NO: 8, wherein at least one amino acid of each of the mutant C. difficile toxins is chemically crosslinked.

In another embodiment, the immunogenic composition includes any sequence selected from SEQ ID NO: 4, SEQ ID NO: 84, and SEQ ID NO: 83, and any sequence selected from SEQ ID NO: 6, SEQ ID NO: 86, and SEQ ID NO: 85. In another embodiment, the immunogenic composition includes SEQ ID NO: 84 and an immunogenic composition including SEQ ID NO: 86. In another embodiment, the immunogenic composition includes SEQ ID NO: 83 and an immunogenic composition including SEQ ID NO: 85. In another embodiment, the immunogenic composition includes SEQ ID NO: 84, SEQ ID NO: 83, SEQ ID NO: 86, and SEQ ID NO: 85.

In another embodiment, the immunogenic composition includes a polypeptide having any one sequence selected from SEQ ID NO: 1 to SEQ ID NO: 761, and a second polypeptide having any one sequence selected from SEQ ID NO: 1 to SEQ ID NO: 761, wherein the polypeptide has been contacted with a chemical crosslinker, such as, for example, formaldehyde or EDC, as described herein. For example, in one embodiment, the immunogenic composition includes a first polypeptide having the amino acid sequence set forth in any one of SEQ ID NOs: 1-8, 15, 17, 19, 21, 23, 25, 28-35, 82-761 and a second polypeptide having the amino acid sequence set forth in any one of SEQ ID NOs: 1-8, 15, 17, 19, 21, 23, 25, 28-35, 82-761, wherein the first polypeptide and the second polypeptide has been contacted with a chemical crosslinker, such as, for example, formaldehyde or EDC, as described herein.

In certain embodiments, it is preferred that the compositions described herein exhibit immunogenic properties (e.g., inducing a detectable and/or neutralizing and/or protective immune response) following appropriate administration to a subject. The presence of neutralizing and/or protective immune response may be demonstrated as described above and/or by showing that infection by a pathogen (e.g., C. difficile) is affected (e.g., decreased) in individuals (e.g., human being or other animal) to whom the materials described herein have been administered as compared to individuals to whom the materials have not been administered. For instance, one or more test subjects (e.g., human or non-human) may be administered by any suitable route and schedule a composition described herein, and then after a suitable amount of time (e.g., about 1 , 2, 3, 4, 5, 6, 7, 8, 9, or 10 weeks) challenged by a pathogenic organism. The animal(s) may be monitored for immune function (e.g., antibody production, T cell activity) following administration and/or challenge. Sera may be analyzed for total antibody response or for expression of particular subtypes using, for example, an antibody ELISA and/or a pathogen neutralization assay. T cell activity may be measured by, for example, measuring IFN-γ production after re-stimulation with the antigen. Statistical analysis (e.g., Fishers exact test, Wilcoxon test, Mann-Whitney Test) may then be performed on data to determine whether the effectiveness of the material in affecting the immune response.

The C. difficile toxoids A and/or B as described herein may be combined with one or more pharmaceutically acceptable carriers to provide a composition prior to administration to a host. A pharmaceutically acceptable carrier is a material that is not biologically or otherwise undesirable, e.g., the material may be administered to a subject, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. Suitable pharmaceutical carriers and their formulations are described in, for example, Remington's: The Science and Practice of Pharmacy, 27⁴′ Edition, David B. Troy, ed., Lippicott Williams & Wilkins (2005), and may be appropriate Typically, an appropriate amount of a pharmaceutically-acceptable salt is used in the formulation to render the formulation isotonic. Examples of the pharmaceutically-acceptable carriers include, but are not limited to, sterile water, saline, buffered solutions like Ringer's solution, and dextrose solution. The pH of the solution is generally from about 5 to about 8 or from about 7 to about 7.5. Other carriers include sustained-release preparations such as semipermeable matrices of solid hydrophobic polymers containing polypeptides or fragments thereof. Matrices may be in the form of shaped articles, e.g., films, liposomes or microparticles. It will be apparent to those persons skilled in the art that certain carriers may be more preferable depending upon, for instance, the route of administration and concentration of composition being administered. Carriers are those suitable for administration to humans or other subjects.

As referred to above, an immunological composition is typically one that comprises C. difficile antigen(s) and, upon administration to a host (e.g., an animal), induces or enhances an immune response directed against the antigen (e.g., C. difficile). Such responses may include the generation of antibodies (e.g., through the stimulation of B cells) or a T cell-based response (e.g., a cytolytic response), as described above, which may be protective and/or neutralizing. A protective or neutralizing immune response may be one that is detrimental to the infectious organism corresponding to the antigen (e.g., from which the antigen was derived) and beneficial to the host (e.g., by reducing or preventing infection). As used herein, protective or neutralizing antibodies and/or cellular responses may be reactive with the C. difficile antigen(s) described here, especially when administered in an effective amount and/or schedule. Those antibodies and/or cellular responses may reduce or inhibit the severity, time, and/or lethality of C. difficile infection when tested in animals. As shown in the examples, the compositions described herein may be used to induce an immune response against C. difficile. An immunological composition that, upon administration to a host, results in a therapeutic (e.g., typically administered during an active infection) and/or protective (e.g., typically administered before or after an active infection) and/or neutralizing immune response, may be considered a vaccine.

In one embodiment, the composition induces an immune response. In a preferred embodiment, use of the composition reduces the incidence of a first primary episode of a C. difficile infection. The incidence may be reduced after the first administration of the composition, after the second administration of the composition, and/or after the third administration of the composition, as compared to the incidence prior to a first administration of the composition. In another embodiment, use of the composition reduces the incidence of recurrent C. difficile infection. The incidence of recurrent infection may be reduced after the first administration of the composition, after the second administration of the composition, and/or after the third administration of the composition.

In another embodiment, use of the composition reduces the severity of a C. difficile infection. For example, the duration of an episode of a C. difficile infection may be reduced after the first administration of the composition, after the second administration of the composition, and/or after the third administration of the composition, as compared to the incidence prior to a first administration of the composition. An episode of a C. difficile infection may include, for example, at least two days of passing at least three unformed stools and/or a need for antibiotic treatment for C. difficile infection. The duration of an episode of a C. difficile infection may be considered reduced if the patient has had at least two days without passage of at least three or more unformed stools and/or there is no further need for antibiotic treatment for C. difficile infection.

In some embodiments, methods for preventing, ameliorating, reducing the risk of and/or treating (e.g., affecting) infection by C. difficile are also provided. Methods for treating one or more disease conditions caused by or involving C. difficile in a subject comprising administering to the subject at least one or more effective doses of a composition described herein (e.g., comprising C. difficile antigens, e.g., toxoid A, toxoid B). The antigens may be administered in a dosage amount of about 1 to about 300 μg (e.g., about any of 1, 2, 3, 4, 5, 6, 7, 8, 9, 10, 15, 20, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290 and/or 300 μg). The antigens may be administered more than once in the same or different dosage amounts. In certain embodiments, the C. difficile antigens may be administered to the subject by the same or different suitable route(s) one, two, three, four, five, six, seven, eight, nine, ten, or more times.

When multiple doses are administered, the doses may comprise about the same or different type and/or amount of C. difficile antigens in each dose. The doses may also be separated in time from one another by the same or different intervals. For instance, the doses may be separated by about any of 6, 12, 24, 36, 48, 60, 72, 84, or 96 hours, seven days, 14 days, 21 days, 30 days, 40 days, 50 days, 60 days, 70 days, 80 days, 90 days, 100 days, 1 10 days, 120 days, 130 days, 140 days, 150 days, 160 days, 170 days, 180 days, 190 days, 200 days, one week, two weeks, three weeks, one month, two months, three months, four months, five months, six months, seven months, eight months, nine months, 10 months, 1 1 months, 12 months, 1.5 years, 2 years, 3 years, 4 years, 5 years, or any time period before, after, and/or between any of these time periods. In some embodiments, the C. difficile antigens may be administered alone or in conjunction with other agents (e.g., antibiotics) Such other agents may be administered simultaneously (or about simultaneously) with the same or different C. difficile antigens, or at a different time and/or frequency. Other embodiments of such methods may also be appropriate as could be readily determined by one of ordinary skill in the art.

Also provided are methods for immunizing a subject (such as a human being) by administering thereto any such compositions. In some embodiments, the methods may comprise administering to the subject an immunogenic composition (e.g., a vaccine) comprising an effective amount (e.g., at least about 40 to about 500, such about 50 to about 100 μg) of C. difficile toxoid A and toxoid B (combined w/w) at an effective toxoid A:B ratio (e.g., 3:1 , 3:2, 1:1 by weight (w/w)), and with a sufficient purity (e.g., at least 90% (w/w)), using one or more administrations (e.g., at least three times, each dose being suitably separated from one another (e.g., at least about 7 days)). An effective toxoid A:B ratio is any ratio that may be included in a composition and induce an effective immune response against C. difficile toxin A and/or toxin B.

In one embodiment, the method may comprise first, second and third administrations wherein the second administration is at least 7 days after the first administration and the third administration is at least about 30 days and/or at least about 180 days after the first and/or second administration.

In some embodiments, the methods may enhance and/or induce an existing immune response in a human being previously exposed to C. difficile (e.g., a seropositive human being, an anamnestic immune response).

In one embodiment, the human has had an unplanned hospitalization within the 12 months prior to the first administration of the composition. In another embodiment, the human has had a skilled nursing facility (a residential institution that provides professional nursing care and rehabilitation services, usually following discharge from hospital) stay within the 12 months prior to the first administration of the composition. In another embodiment, the human has had a nursing home (e.g., a residential institution that provides professional nursing care and rehabilitation services, usually following discharge from a hospital) stay within the 12 months prior to the first administration of the composition. In another embodiment, the human has had two or more emergency room visits within the 12 months prior to the first administration of the composition. In another embodiment, the human has had 10 or more out-patient visits (primary and/or secondary care visits but excluding pharmacy and mental health visits) within the 12 months prior to the first administration of the composition.

In another embodiment, the human has been administered systemic antibiotic use within the 12 weeks prior to the first administration of the composition. In another embodiment, the human has a significant co-morbidity or contact with health care systems within the 12 months prior to the first administration of the composition. In another embodiment, the human has had 1 in-patient hospitalization nights within the 12 months prior to the first administration of the composition. In another embodiment, the human has had 2 emergency room visits within the 12 months prior to the first administration of the composition. In another embodiment, the human has had 10 out-patient visits within the 12 months prior to the first administration of the composition. In another embodiment, the human has a residence in a skilled nursing facility within the 12 months prior to the first administration of the composition. In another embodiment, the human has a residence in a nursing home within the 12 months prior to the first administration of the composition. In another embodiment, the human has an in-patient hospitalization nights scheduled 37 days after randomization within the 12 months prior to the first administration of the composition. In another embodiment, the human has received systemic antibiotics at any time within the previous 12 weeks prior to the first administration of the composition.

In certain embodiments, the human works at or has contact with any one of the following facilities within the 12 months prior to the first administration of the composition: a hospital, skilled nursing facility (a residential institution that provides professional nursing care and rehabilitation services, usually following discharge from hospital), a nursing home (e.g., a residential institution that provides professional nursing care and rehabilitation services, usually following discharge from a hospital), emergency room, and out-patient facility (primary and/or secondary care visits but excluding pharmacy and mental health visits).

In certain embodiments, human being(s) may have had, in the 12 month period before the first administration, at least one or two hospital stays, each lasting at least about 24, 48 or 72 hours or more, and/or had received systemic (not topical) antibiotics; and/or, is anticipated to have an in-patient hospitalization for a planned surgical procedure within about 60 days of the first administration. In some embodiments, the anticipated/impending hospital stay/hospitalization may be planned to be for about 24, 48 to 72 hours or more and may be for a surgery involving at least one of the kidney/bladder/urinary system, musculoskeletal system, respiratory system, circulatory system, and central nervous system.

It is preferred that the immune response elicited by these methods is sufficient to prevent and/or ameliorate and/or reduce the risk of symptomatic C. difficile infection. In certain embodiments, the method may comprise administering the immunogenic composition to a human subject at risk for a symptomatic infection that is at least about 40, 50 or 65, 70, 75, 80, or 85 years of age. In some embodiments, the method may comprise administering the composition to each individual of a group aged between about 40 and about 65 years old and/or between about 65 and about 75 years old. In some embodiments, the method may induce about a two- to four-fold enhancement of an antibody-based immune response against C. difficile toxin A and/or toxin B in about any of 80, 85, 90, 95 or 100% of a population of individuals considered seropositive before the first administration as measured by, e.g., ELISA and/or TNA. In some embodiments, the method may induce about a two- to four-fold enhancement of an antibody-based immune response against C. difficile toxin A and/or toxin B in about any of 20, 25, 30, 35, 40, 45, or 50% of a population of individuals considered seronegative before administration of the composition, as measured by, e.g., ELISA and/or TNA 14 days after the first administration (e.g., following administration at days 0, seven and 30). In some embodiments, the method may induce about a two- to four-fold enhancement of an antibody-based immune response against C. difficile toxin A and/or toxin B in about any of 30, 35, 40, 45, 50, 55, 60, 65, 70, 75, or 80% of a population of individuals considered seronegative before administration of the composition, as measured by, e.g., ELISA and/or TNA 60 days after the first administration (e.g., following administration at days 0, seven and 30). In some embodiments, the individuals in such populations are from about 40 to about 65 years old. In some embodiments, the individuals in such populations are from about 50 to 75 years old or about 50 years old to about 65 years old. In some embodiments, this enhancement is observed about 30 days after the first administration (at day 0), typically follows a second administration at about day 7, and is typically observed before the third administration (at, e.g., about day 30 or day 180). In some embodiments, the immune response may be detectable against toxin A and/or toxin B for up to about 30 months (e.g., about 1000 days) after the first, second and/or third administration in a multiple regimen administration protocol. In some embodiments, administration of a composition described herein to a human subject at day 0 (first administration), about day 7 (second administration) and about day 30 (third administration) enhances or induces an immune response against C. difficile toxin A and/or toxin B for up to about 30 months, or about 1000 days as measured by, e.g., ELISA and/or TNA, preferably by a cytoxicity assay. In some embodiments, the level of the immune response may be about at least as high on about day 1000 following the first administration as on about day 14 following the first administration of a three dose administration regimen, as measured by, e.g., ELISA and/or TNA, preferably by a cytoxicity assay. In some embodiments, the level of the immune response may be about at least as high on about any of days 100, 200, 300, 400, 500, 600, 700, 800, 900 and 1000 following the first administration as on about day 14 following the first administration as measured by, e.g., ELISA and/or TNA, preferably by a cytoxicity assay. In some embodiments, the immune response may be about two- to eight-fold above baseline (e.g., antitoxin A and/or toxin B antibody levels at day 0, before the first administration, as measured by e.g., ELISA and/or TNA. In some embodiments, the immune response may be from about 2.5 to about 6.8-fold above baseline as measured by e.g., ELISA and/or TNA, preferably by a cytoxicity assay. In some embodiments, the immune response in seropositive individuals (e.g., non-naive) is increased from baseline by a factor of about three at about day 7; about 10 to about 70 at about day 14; about 30 to about 200 at about day 30; and about 100 to about 200 at about day 60, as measured by ELISA for toxins A and/or B (e.g., following administration at days 0, 7 and 30). In some embodiments, the immune response in seropositive individuals (e.g., non-naive) is increased from baseline by a factor of about three at about day 7; about 10 to about 100 at about day 14; about 1 5 to about 1 30 at about day 30; and about 100 to about 130 at about day 60, as measured by TNA for toxins A and/or B (e.g., following administration at days 0, seven and 30). In some embodiments, the immune response in seronegative individuals (e.g., naive) is increased from baseline by a factor of about two at about day 14; about five to about 10 at about day 30; and about 25 to about 60 at about day 60, as measured by ELISA for toxins A and/or B (e.g., following administration at days 0, seven and 30). In some embodiments, the immune response in seronegative individuals (e.g., naive) is increased from baseline by a factor of about two to about three at about day 14; about two to about five at about day 30; and about five to about 40 at about day 60, as measured by TNA for toxins A and/or B (e.g., following administration at days 0, 7 and 30). In some embodiments, the immune responses described herein are detected in individuals considered either seropositive or seronegative at day 0 (e.g., before the first administration). In some embodiments, such immune response is detected for both C. difficile toxin A and toxin B as measured by, e.g., ELISA and/or TNA, preferably by a cytoxicity assay. Methods (e.g., in vitro or in vivo) for producing such C. difficile antigens (e.g., toxoids A and/or B), and compositions comprising the same, are also provided. Such methods may include, for example, any of those available and/or known to those of ordinary skill in the art, and/or the methods described in WIPO Patent Application WO/2012/143902, U.S. Pat. No. 9,187,536, and WIPO Patent Application WO/2014/060898, which are each incorporated by reference herein in their respective entireties.

As used herein, a subject or a host is meant to be an individual. The subject can include domesticated animals, such as cats and dogs, livestock (e.g., cattle, horses, pigs, sheep, and goats), laboratory animals (e.g., mice, rabbits, rats, guinea pigs) and birds. In one aspect, the subject is a mammal such as a primate or a human.

### Composition and Vaccine

In one embodiment, the composition is an immunogenic composition. In one embodiment, the composition is an immunogenic composition for a human. In another embodiment, the composition is a vaccine. A “vaccine” refers to a composition that includes an antigen, which contains at least one epitope that induces an immune response that is specific for that antigen. The vaccine may be administered directly into the subject by subcutaneous, oral, oronasal, or intranasal routes of administration. Preferably, the vaccine is administered intramuscularly. In one embodiment, the composition is a human vaccine. In one embodiment, the composition is an immunogenic composition against C. difficile.

In certain embodiments, the compositions may further comprise one or more C. difficile antigens, one or more pharmaceutically acceptable carriers and/or one or more adjuvants (e.g., aluminum salt, emulsion, cationic liposome, anionic polymer, Toll-like receptor agonist, and a combination thereof).

In one embodiment, the composition, which may be a vaccine, may be provided as a lyophilized formulation that may be reconstituted at the clinical site with diluent, and mixed with either adjuvant (e.g., an aluminum adjuvant such as aluminum phosphate or aluminum hydroxide or water for injection (WFI), when specified.

In one embodiment, the composition includes a pharmaceutically acceptable carriers, which refer to any solvents, dispersion media, stabilizers, diluents, and/or buffers that are physiologically suitable. Exemplary stabilizers include carbohydrates, such as sorbitol, mannitol, starch, dextran, sucrose, trehalose, lactose, and/or glucose; inert proteins, such as albumin and/or casein; and/or other large, slowly metabolized macromolecules, such as polysaccharides such as chitosan, polylactic acids, polyglycolic acids and copolymers (such as latex functionalized SEPHAROSE™ agarose, agarose, cellulose, etc.), amino acids, polymeric amino acids, amino acid copolymers, and lipid aggregates (such as oil droplets or liposomes). Additionally, these carriers may function as immunostimulating agents (i.e., adjuvants).

Preferably, the composition includes trehalose. Preferred amounts of trehalose (% by weight) include from a minimum of about 1%, 2%, 3%, or 4% to a maximum of about 10%, 9%, 8%, 7%, 6%, or 5%. Any minimum value can be combined with any maximum value to define a suitable range. In one embodiment, the composition includes about 3%-6% trehalose, most preferably, 4.5% trehalose, for example, per 0.5 mL dose.

Examples of suitable diluents include distilled water, saline, physiological phosphate-buffered saline, glycerol, alcohol (such as ethanol), Ringer's solutions, dextrose solution, Hanks' balanced salt solutions, and/or a lyophilization excipient. The diluent may be, for example, any pharmaceutically acceptable diluent (e.g., 20 mM Sodium Citrate, 5% Sucrose, and 0.016% Formaldehyde; 10 niM Citrate, 4% Sucrose, 0.008% Formaldehyde, 0.57% Sodium Chloride). In a preferred embodiment, the composition includes 10 mM Tris, 4.5%, Trehalose, 0.01% Polysorbate 80 (PS80), pH 7.4

Exemplary buffers include phosphate (such as potassium phosphate, sodium phosphate); acetate (such as sodium acetate); succinate (such as sodium succinate); glycine; histidine; carbonate, Tris (tris(hydroxymethyl)aminomethane), and/or bicarbonate (such as ammonium bicarbonate) buffers. Preferably, the composition includes tris buffer. Preferred amounts of tris buffer include from a minimum of about 1 mM, 5 mM, 6 mM, 7 mM, 8 mM, 9 mM, 10 mM to a maximum of about 100 mM, 50 mM, 20 mM,19 mM, 18 mM, 17 mM, 16 mM, 15 mM, 14 mM, 13 mM, 12 mM, or 11 mM. Any minimum value can be combined with any maximum value to define a suitable range. In one embodiment, the composition includes about 8 mM to 12 mM tris buffer, most preferably, 10 mM tris buffer, for example, per 0.5 mL dose.

In another preferred embodiment, the composition includes histidine buffer. Preferred amounts of histidine buffer include from a minimum of about 1 mM, 5 mM, 6 mM, 7 mM, 8 mM, 9 mM, 10 mM to a maximum of about 100 mM, 50 mM, 20 mM,19 mM, 18 mM, 17 mM, 16 mM, 15 mM, 14 mM, 13 mM, 12 mM, or 11 mM. Any minimum value can be combined with any maximum value to define a suitable range. In one embodiment, the composition includes about 8 mM to 12 mM histidine buffer, most preferably, 10 mM histidine buffer, for example, per 0.5 mL dose.

In yet another preferred embodiment, the composition includes phosphate buffer. Preferred amounts of phosphate buffer include from a minimum of about 1 mM, 5 mM, 6 mM, 7 mM, 8 mM, 9 mM, 10 mM to a maximum of about 100 mM, 50 mM, 20 mM,19 mM, 18 mM, 17 mM, 16 mM, 15 mM, 14 mM, 13 mM, 12 mM, or 11 mM. Any minimum value can be combined with any maximum value to define a suitable range. In one embodiment, the composition includes about 8 mM to 12 mM phosphate buffer, most preferably, 10 mM phosphate buffer, for example, per 0.5 mL dose.

The pH of the buffer will generally be chosen to stabilize the active material of choice, and can be ascertainable by those in the art by known methods. Preferably, the pH of the buffer will be in the range of physiological pH. Thus, preferred pH ranges are from about 3 to about 8; more preferably, from about 6.0 to about 8.0; yet more preferably, from about 6.5 to about 7.5; and most preferably, at about 7.0 to about 7.2.

In another embodiment, the compositions described herein may include an adjuvant, as described below. Preferred adjuvants augment the intrinsic immune response to an immunogen without causing conformational changes in the immunogen that may affect the qualitative form of the immune response. Exemplary adjuvants include 3 De-O-acylated monophosphoryl lipid A (MPL™) (see GB 2220211 (GSK)); an aluminum hydroxide gel such as ALHYDROGEL™ (Brenntag Biosector, Denmark); aluminum salts (such as aluminum hydroxide, aluminum phosphate, aluminum sulfate), which may be used with or without an immunostimulating agent such as MPL or 3-DMP, QS-21, polymeric or monomeric amino acids such as polyglutamic acid or polylysine. Yet another exemplary adjuvant is an immunostimulatory oligonucleotide such as a CpG oligonucleotide (see, e.g., WO 1998/040100, WO2010/067262), ora saponin and an immunostimulatory oligonucleotide, such as a CpG oligonucleotide (see, e.g., WO 00/062800). In a preferred embodiment, the adjuvant is a CpG oligonucleotide, most preferably a CpG oligodeoxynucleotides (CpG ODN). Preferred CpG ODN are of the B Class that preferentially activate B cells. In aspects of the invention, the CpG ODN has the nucleic acid sequence 5′ T*C*G*T*C*G*T*T*T*T*T*C*G*G*T*G*C*T*T*T*T 3′ (SEQ ID NO: 48) wherein * indicates a phosphorothioate linkage. The CpG ODN of this sequence is known as CpG 24555, which is described in WO2010/067262. In a preferred embodiment, CpG 24555 is used together with an aluminium hydroxide salt such as ALHYDROGEL. A further class of exemplary adjuvants include saponin adjuvants, such as STIMULON™ (QS-21, which is a triterpene glycoside or saponin, Aquila, Framingham, Mass.) or particles generated therefrom such as ISCOMs (immune stimulating complexes) and ISCOMATRIX® adjuvant. Accordingly, the compositions of the present invention may be delivered in the form of ISCOMs, ISCOMS containing CTB, liposomes or encapsulated in compounds such as acrylates or poly(DL-lactide-co-glycoside) to form microspheres of a size suited to adsorption. Typically, the term “ISCOM” refers to immunogenic complexes formed between glycosides, such as triterpenoid saponins (particularly Quil A), and antigens which contain a hydrophobic region. In a preferred embodiment, the adjuvant is an ISCOMATRIX adjuvant. Other exemplary adjuvants include RC-529, GM-CSF and Complete Freund's Adjuvant (CFA) and Incomplete Freund's Adjuvant (IFA). Yet another class of exemplary adjuvants is glycolipid analogues including N-glycosylamides, N-glycosylureas and N-glycosylcarbamates, each of which is substituted in the sugar residue by an amino acid. Optionally, the pharmaceutical composition includes two or more different adjuvants. Preferred combinations of adjuvants include any combination of adjuvants including, for example, at least two of the following adjuvants: alum, MPL, QS-21, ISCOMATRIX, CpG, and ALHYDROGEL. An exemplary combination of adjuvants includes a combination of CpG and ALHYDROGEL.

The adjuvant may comprise, for instance, a suitable concentration (e.g., about any of 800-1600 μg/mL) of an adjuvant, such, as an adjuvant comprising aluminum (e.g., aluminum hydroxide or aluminum phosphate) in WFI. For instance, the adjuvant (e.g., 800-1600 g/mL aluminum hydroxide in 0.57% Sodium Chloride) may be used as the diluent to reconstitute the lyophilized formulation. WFI may be used to dilute the lyophilized vaccine for the unadjuvanted formulations. The final dosing solution may comprise, for instance, composition/vaccine, diluent and adjuvant.

Alternatively, in one embodiment, the composition is administered to the mammal in the absence of an adjuvant. That is, the composition does not comprise an adjuvant.

In some embodiments, the composition includes a surfactant. Any surfactant is suitable, whether it is amphoteric, non-ionic, cationic or anionic. Exemplary surfactants include the polyoxyethylene sorbitan esters surfactants (e.g., TWEEN®), such as polysorbate 20 and/or polysorbate 80; polyoxyethylene fatty ethers derived from lauryl, cetyl, stearyl and oleyl alcohols (known as BRIJ surfactants), such as triethyleneglycol monolauryl ether (BRIJ 30); TRITON X 100, or t-octylphenoxypolyethoxyethanol; and sorbitan esters (commonly known as the SPANs), such as sorbitan trioleate (SPAN 85) and sorbitan monolaurate, and combinations thereof. Preferred surfactants include polysorbate 80 (polyoxyethylene sorbitan monooleate).

Polysorbate 80 (PS-80) is a non-ionic surfactant. In one embodiment, the composition includes a PS-80 concentration ranging from 0.0005% to 1%. For example, the PS-80 concentration in the composition may be at least 0.0005%, 0.005%, 0.01%, 0.02%, 0.03%, 0.04%, 0.05%, 0.06%, 0.07%, 0.08%, 0.09%, 0.10%, 0.2%, 0.3%, 0.4%, 0.5%, 0.6%, 0.7%, 0.8%, 0.9%, 1%, or 1.1% PS-80. In one embodiment, the PS-80 concentration in the composition may be at most 2.0%, 1.9%, 1.8%, 1.7%, 1.6%, 1.5%, 1.4%, 1.3%, 1.2%, 1.1%, 1.0%, 0.9%, 0.8%, or 0.7% PS-80. Any minimum value may be combined with any maximum value described herein to define a range. Preferably, the composition comprises 0.01% PS-80.

In an exemplary embodiment, the immunogenic composition includes trehalose and phosphate 80. In another exemplary embodiment, the immunogenic composition includes tris buffer and polysorbate 80. In another exemplary embodiment, the immunogenic composition includes histidine buffer and polysorbate 80. In yet another exemplary embodiment, the immunogenic composition includes phosphate buffer and polysorbate 80.

In one exemplary embodiment, the immunogenic composition includes trehalose, tris buffer and polysorbate 80. In another exemplary embodiment, the immunogenic composition includes trehalose, histidine buffer and polysorbate 80. In yet another exemplary embodiment, the immunogenic composition includes trehalose, phosphate buffer and polysorbate 80.

In some embodiments, the pharmaceutical composition further includes formaldehyde. For example, in a preferred embodiment, a pharmaceutical composition that further includes formaldehyde has an immunogenic composition, wherein the mutant C. difficile toxin of the immunogenic composition has been contacted with a chemical crosslinking agent that includes formaldehyde. The amount of formaldehyde present in the pharmaceutical composition may vary from a minimum of about 0.001%, 0.002%, 0.003%, 0.004%, 0.005%, 0.006%, 0.007%, 0.008%, 0.009%, 0.010%, 0.013%, or 0.015%, to a maximum of about 0.020%, 0.019%, 0.018%, 0.017% 0.016%, 0.015%, 0.014%, 0.013%, 0.012% 0.011% or 0.010%. Any minimum value can be combined with any maximum value to define a suitable range. In one embodiment, the pharmaceutical composition includes about 0.010% formaldehyde.

In some alternative embodiments, the pharmaceutical compositions described herein do not include formaldehyde. For example, in a preferred embodiment, a pharmaceutical composition that does not include formaldehyde has an immunogenic composition, wherein at least one amino acid of the mutant C. difficile toxin is chemically crosslinked by an agent that includes EDC. More preferably, in such an embodiment, the mutant C. difficile toxin has not been contacted with a chemical crosslinking agent that includes formaldehyde. As another exemplary embodiment, a pharmaceutical composition that is in a lyophilized form does not include formaldehyde.

Also provided herein are kits for administering the C. difficile antigens. In one embodiment, one or more of C. difficile antigens may form part of and/or be provided as a kit for administration to a subject. Instructions for administering the C. difficile antigens may also be provided by the kit. Compositions comprising C. difficile antigens as described herein may be included in a kit (e.g., a vaccine kit). For example, the kit may comprise a first container containing a composition described herein in dried or lyophilized form and a second container containing an aqueous solution for reconstituting the composition. The kit may optionally include the device for administration of the reconstituted liquid form of the composition (e.g., hypodermic syringe, microneedle array) and/or instructions for use. The device for administration may be supplied pre-filled with an aqueous solution for reconstituting the composition.

The volume of each delivered dose of study drug (vaccine or placebo) may be about 0.5 mL. The volume of each delivered dose of the composition disclosed herein may be about 0.2, 0.3, 0.4, 0.5, 0.6, 0.7; 0.8, 0.9 or 1 mL. The volume of each delivered dose of the composition disclosed herein may be about 0.4, 0.5, 0.6 ml. The volume of each delivered dose of the composition disclosed herein may be about 0.5 mL. The volume of each delivered dose of the composition disclosed herein may be about 1 mL. Formulations may be administered by any suitable route (e.g., subcutaneously, intravenously, intramuscularly, intraperitoneally, intradermally, intranodally, intranasally, orally).

### Toxin Neutralizing Activity

Immune response induced by administering the composition to a human may be determined using a toxin neutralization assay (TNA), ELISA, or more preferably, a cytotoxicity assay, such as that described in WIPO Patent Application WO/2012/143902, U.S. Pat. No. 9,187,536, and WIPO Patent Application WO/2014/060898, which are each incorporated by reference herein in their respective entireties.

The in vitro cytotoxicity assay is a key safety assay developed for testing of any potential residual cytotoxicity in drug substance material. Measurement of any potential residual cytotoxicity in toxoid A is accomplished using an IMR-90 cell-based assay. The wild-type C. difficile toxin A exhibits potent in vitro cytotoxicity, with small amounts of the toxin being sufficient to cause various effects on mammalian cells such as cell rounding (cytopathic effect or CPE) and lack of metabolic activity (as measured by ATP levels). The CPE assay is conducted by incubating toxoid material with the IMR90 cells in culture at 500 mcg/mL at 37° C., and evaluating for cell rounding 24 hours later. The CPE assay requires a subjective visual assessment of CPE by trained analysts and thus cannot be easily validated. The cytotoxicity release assay has been developed based on measurement of the amount of luminescence signal generated from ATP, which is proportional to the number of metabolically active cells following treatment with either toxoid A or wild-type toxin. The results are expressed as EC50, which is defined as the amount of toxin or toxoid that causes a 50% reduction in ATP levels as measured in relative light units. The toxoid is tested at a concentration of 100 mcg/mL. This method was chosen for release and stability (limited time points) testing because it is more robust, objective, and suitable for GMP testing than an alternative cytopathogenic effect (CPE) assay. The cytotoxicity assay is run only on the toxoid because it can be tested at a higher concentration as compared to the drug product material without matrix interference. This ensures that the measurement is made at the most concentrated stage during the C. difficile vaccine production cycle. In addition, the cytotoxicity assay will be conducted on stability to monitor any potential reversion to toxicity.

The in vitro cytotoxicity assay is a key safety assay developed for testing of any potential residual cytotoxicity in drug substance material. Measurement of any potential residual cytotoxicity in toxoid B is accomplished using an IMR-90 cell-based assay. The wild-type C. difficile toxin B exhibits potent in vitro cytotoxicity, with small amounts of the toxin being sufficient to cause various effects on mammalian cells such as cell rounding (cytopathic effect or CPE) and lack of metabolic activity (as measured by ATP levels). The CPE assay is conducted by incubating DS material with the IMR90 cells in culture at 500 mcg/mL at 37° C., and evaluating for cell rounding 24 hours later. The CPE assay requires a subjective visual assessment of CPE by trained analysts and thus cannot be easily validated. The cytotoxicity release assay has been developed based on measurement of the amount of luminescence signal generated from ATP, which is proportional to the number of metabolically active cells following treatment with either toxoid B or wild-type toxin B. The results are expressed as EC50, which is defined as the amount of toxin or toxoid that causes a 50% reduction in ATP levels as measured in relative light units. The maximum concentration of toxoid that was originally tested in this assay was 200 mcg/mL. However, method performance over time suggested that an upper concentration of only 100 mcg/mL can be consistently supported. This method was chosen for release and stability (limited time points) testing because it is more robust, objective, and suitable for GMP testing than an alternative CPE assay. The cytotoxicity assay is run only on the toxoid B drug substance material because it can be tested at a higher concentration as compared to the drug product material without matrix interference. This ensures that the measurement is made at the most concentrated stage during the C. difficile vaccine production cycle. In addition, the cytotoxicity assay will be conducted on stability to monitor any potential reversion to toxicity.

To qualify the 50% neutralization titer assay for clinical use, provide further robustness to the assay, and assure consistent long-term performance in clinical development, a reference standard and appropriate controls were added to the assay, thereby permitting the read out of a neutralization titer as neutralization units/mL defined by the reference standard. Prior to analysis of the current study, serum samples from vaccinated humans were used to demonstrate a linear relationship between 50% neutralization titers and neutralization units/mL when performing the neutralization assay. Based on these correlation studies, “protective” neutralization threshold values were calculated and used to analyze the clinical data in this study.

In one embodiment, the TNA is an automated and sensitive assay based on luminescence readout. Neutralization titers of test samples are calculated based on a Reference standard. In one embodiment, the assay LLOQ for Txd A is 158.0 U/ml; Txd B=249.5 U/ml. Preferably, the TNA

For the immunogenicity analyses, the “protective” thresholds for antitoxin A- and toxin B-neutralizing antibody responses were 219 and 2586 neutralization units/mL, respectively. Several of the immunogenicity endpoints for Study B5091009 were assessed based upon these “protective” thresholds.

As used herein, unless expressly defined otherwise, the “specified threshold” value is defined as 219 neutralization units/mL for toxin A and 2586 neutralization units/mL for toxin B.

In one embodiment, the immune response induced in the human is neutralizing against a C. difficile strain that expresses a toxin A having an amino acid sequence that has at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the toxoid A of the composition.

In another embodiment, the immune response induced in the human is neutralizing against a C. difficile strain that expresses a toxin B including an amino acid sequence that has at least 80%, 81%, 82%, 83%, 84%, 85%, 86%, 87%, 88%, 89%, 90%, 91%, 92%, 93%, 94%, 95%, 96%, 97%, 98%, 99%, or 100% identity to the toxoid B of the composition.

The usefulness (e.g., immunogenicity) of any of the materials (e.g., compositions) and/or methods described herein may be assayed by any of the variety of methods known to those of skill in the art. Any one or more of the assays described herein, or any other one or more suitable assays, may be used to determine the suitability of any of the materials described herein for an intended purpose. It is to be understood that these methods are exemplary and non-limiting; other assays may also be suitable. For instance, the compositions described herein typically induce and/or enhance the production of antibodies against C. difficile upon administration to a subject. Such antibodies may be detected in the subject using any of the methods available to those of ordinary skill in the art. For instance, as described in the

Examples section, serum may be obtained from a subject and tested by ELISA to detect immunoglobulin type G (IgG) antibodies to C. difficile toxin A and/or toxin B (e.g., “primary immunogenicity data”). Antibodies present in test sera may be reacted with toxin A or B antigens adsorbed to individual wells of a microtiter plate. The amount of antibody bound to the antigen coated wells may be determined using a colorimetric substrate reaction after binding of a secondary anti-IgG (e.g., anti-human IgG) antibody-enzyme conjugate. Substrate for the enzyme is then typically added that causes colorimetric change that was directly proportional to the antibody bound to the antigen. The concentration of antibodies in serum may be derived by extrapolation from a standard curve, which was generated from multiple dilutions of a reference standard serum with defined IgG units (ELISA unit (EU)/mL)). A toxin neutralization assay (TNA) may also be used to quantitate neutralizing antibodies to C. difficile toxin. In this assay, serial diluted serum may be incubated with a fixed amount of C. difficile toxin A or B. Test cells (e.g., Vero cells) may then then added and serum-toxin-cell mixture incubated under appropriate conditions (e.g., 37° C. for 6 days). The ability of the sera to neutralize the cytotoxic effect of the C. difficile toxin may be determined by and correlated to the viability of the cells. The assay utilizes the accumulation of acid metabolites in closed culture wells as an indication of normal cell respiration. In cells exposed to toxin, metabolism and CO₂ production is reduced; consequently, the pH rises (e.g., to 7.4 or higher) as indicated by the phenol red pH indicator in the cell culture medium. At this pH, the medium appears red. Cell controls, or cells exposed to toxin which have been neutralized by antibody, however, metabolize and produce CO₂ in normal amounts; as a result, the pH is maintained (e.g., at 7.0 or below) and at this pH, the medium appears yellow. Therefore, C. difficile toxin neutralizing antibodies correlate with the ability of the serum to neutralize the metabolic effects of C. difficile toxin on cells as evidenced by their ability to maintain a certain pH (e.g., of 7.0 or lower). The color change of the media may be measured (e.g., at 562 nm to 630 nm) using a plate reader to further calculate the antitoxin neutralizing antibody titer at 50% inhibition of the C. difficile toxin-mediated cytotoxicity. In one embodiment, the composition induces a toxin neutralizing antibody titer that is at least greater than 1-fold, such as, for example, at least 1.01-fold, 1.1-fold, 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 32-fold, or higher in the human after receiving a dose of the composition than a toxin neutralizing antibody titer in the human prior to receiving said dose, when measured under identical conditions in a toxin neutralization assay.

### Titers

In one embodiment, the composition induces an increase in toxin neutralizing antibody titer in the human, as compared to the toxin neutralizing antibody titer in the human prior to administration of a dose of the composition, when measured under identical conditions in, for example, a cytotoxicity assay. In one embodiment, the increase in toxin neutralizing titer is compared to the toxin neutralizing titer in the human before administration of the first dose of the composition, as compared to the toxin neutralizing titer in the human prior to administration of the first dose of the composition, when measured under identical conditions in, for example, a cytotoxicity assay. In another embodiment, the increase in titer is observed after a second dose of the composition, as compared to the toxin neutralizing titer in the human prior to administration of the first dose of the composition, when measured under identical conditions in, for example, a cytotoxicity assay. In another embodiment, the increase in toxin neutralizing titer is observed after a third dose of the composition, as compared to the toxin neutralizing titer in the human prior to administration of the first dose of the composition, when measured under identical conditions in, for example, a cytotoxicity assay. In another embodiment, the increase in titer is observed after a second dose of the composition, as compared to the toxin neutralizing titer in the human prior to administration of the second dose of the composition, when measured under identical conditions in, for example, a cytotoxicity assay. In another embodiment, the increase in toxin neutralizing titer is observed after a third dose of the composition, as compared to the toxin neutralizing titer in the human prior to administration of the third dose of the composition, when measured under identical conditions in, for example, a cytotoxicity assay.

In one embodiment, the composition induces a toxin neutralizing titer in the human after administration of a dose, wherein the toxin neutralizing titer is at least greater than 1-fold higher than the toxin neutralizing titer in the human prior to administration of the dose, when measured under identical conditions in, for example, a cytotoxicity assay. For example, the toxin neutralizing titer may be at least 1.01-fold, 1.1-fold, 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 32-fold, or 64-fold higher in the human after receiving a dose of the composition, as compared to the toxin neutralizing titer in the human prior to administration of the dose, when measured under identical conditions in, for example, a cytotoxicity assay.

In one embodiment, a “responder” refers to a human, wherein the composition induces a toxin neutralizing titer in the human after administration of a dose, wherein the toxin neutralizing titer is at least greater than 1-fold higher than the toxin neutralizing titer in the human prior to administration of the dose. In a preferred embodiment, the responder achieves at least a ≥4-fold rise in toxin neutralizing titer, as compared to a toxin neutralizing titer in the human prior to administration of the dose. Such a responder may be referred to as having a protective titer.

In one embodiment, the composition induces a toxin neutralizing titer in the human after receiving the first dose that is at least 2-fold higher than the toxin neutralizing titer in the human prior to receiving the first dose (e.g., higher than the toxin neutralizing titer in the human in the absence of the first dose), when measured under identical conditions in the cytotoxicity assay. In one embodiment, the composition induces a toxin neutralizing titer in the human that is at least 4-fold higher than the toxin neutralizing titer in the human prior to receiving the first dose, when measured under identical conditions in a cytotoxicity assay. In one embodiment, the composition induces a toxin neutralizing titer in the human that is at least 8-fold higher than the toxin neutralizing titer in the human prior to receiving the first dose, when measured under identical conditions in a cytotoxicity assay.

In one embodiment, the human has, for example, a toxin neutralizing titer equal to or greater than the lower limit of quantitation (LLOQ) of the cytotoxicity assay after administration of the first dose of the composition. In another embodiment, the human has, for example, a cytotoxicity assay titer equal to or greater than the LLOQ of the cytotoxicity assay after administration of the second dose of the composition. In another embodiment, the human has, for example, a toxin neutralizing titer equal to or greater than the LLOQ of the cytotoxicity assay after administration of the third dose of the composition.

### Methods and Administration

In one aspect, the invention relates to a method of inducing an immune response against C. difficile in a human. In another aspect, the invention relates to a method of vaccinating a human. In one embodiment, the method includes administering to the human at least one dose of the composition described above. In a preferred embodiment, the method includes administering to the human at most one dose of the composition described above. In another embodiment, the method includes administering to the human at least a first dose and a second dose of the composition described above.

In one embodiment, the second dose is administered at least 20, 30, 50, 60, 100, 120, 160, 170, or 180 days after the first dose, and at most 250, 210, 200, or 190 days after the first dose. Any minimum value may be combined with any maximum value described herein to define a range.

In another embodiment, the second dose is administered about 30 days after the first dose. In another embodiment, the second dose is administered about 60 days after the first dose, such as, for example, in a 0, 2 month immunization schedule. In another embodiment, the second dose is administered about 180 days after the first dose, such as, for example, in a 0, 6 month immunization schedule. In yet another embodiment, the second dose is administered about 120 days after the first dose, such as, for example, in a 2, 6 month immunization schedule.

In one embodiment, the method includes administering to the human two doses of the composition and at most two doses. In one embodiment, the two doses are administered within a period of about 6 months after the first dose. In one embodiment, the method does not include further administration of a booster to the human. A “booster” as used herein refers to an additional administration of the composition to the human. Administering to the human at most two doses of the composition may be advantageous. Such advantages include, for example, facilitating a human to comply with a complete administration schedule and facilitating cost-effectiveness of the schedule.

In one embodiment, the first dose and the second dose are administered to the human over a period of about 5 days, 7 days, 14 days, 21, 25, 30, 40, 50, 60, 70, 80, 90, 100, 110, 120, 130, 140, 150, 160, 170, 180, 190, or 200 days, and most 400, 390, 380, 370, 365, 350, 340, 330, 320, 310, 300, 290, 280, 270, 260, 250, 240, 230, 220, 210, or 200 days after the first dose. In one embodiment, the second dose is administered to the human at least 8, 14, 21, 25, or 30 days and at most 100, 90, 80, 70, 60, 50, 45, 40, 35, or 30 days after administration of the first dose. For example, in one embodiment, the second dose is administered to the human at least 21 days and at most 40 days after administration of the first dose. Any minimum value may be combined with any maximum value described herein to define a range. Preferably, the first dose and second dose are administered to the human over a period of about 8 days. More preferably, the first dose and second dose are administered to the human over a period of about 30 days. Most preferably, the first dose and second dose are administered to the human over a period of at least about 30 days.

In one embodiment, the first dose and the second dose are administered to the human over a period of about 30 days. In another embodiment, the first dose and the second dose are administered to the human over a period of about 60 days. In another embodiment, the first dose and the second dose are administered to the human over a period of about 180 days.

In one embodiment, the first dose and the second dose are administered to the human over a period of at most 30 days. In another embodiment, the first dose and the second dose are administered to the human over a period of at most 60 days. In another embodiment, the first dose and the second dose are administered to the human over a period of at most 180 days.

### Doses

In one embodiment, the method includes administering to the human three doses of the composition. In another embodiment, the method includes administering at most three doses of the composition. In one embodiment, the three doses are administered within a period of about 6 months after the first dose. In one embodiment, the method includes an administration of a booster dose to the human after the third dose. In another embodiment, the method does not include administration of a booster dose to the human after the third dose. In another embodiment, the method does not further include administering a fourth or booster dose of the composition to the human. In a further embodiment, at most three doses within a period of about 6 months are administered to the human.

In an exemplary embodiment, the second dose is administered about 30 days after the first dose, and the third dose is administered about 150 to 180 days after the second dose, such as, for example, in a 0, 1, 6 month immunization schedule. In another exemplary embodiment, the second dose is administered about 60 days after the first dose, and the third dose is administered about 120 days after the second dose, such as, for example, in a 0, 2, 6 month immunization schedule.

In one embodiment, the first dose, second dose, and third dose are administered to the human over a period of about 150, 160, 170, or 180 days, and at most 240, 210 200, or 190 days. Any minimum value may be combined with any maximum value described herein to define a range. Preferably, the first dose, second dose, and third dose is administered to the human over a period of about 180 days or 6 months. For example, the second dose may be administered to the human about 60 days after the first dose, and the third dose may be administered to the human about 120 days after the second dose. Accordingly, an exemplary schedule of administration includes administering a dose to the human at about months 0, 2, and 6.

As described above, multiple doses of the immunogenic composition may be administered to the human, and the number of days between each dose may vary. An advantage of the method includes, for example, flexibility for a human to comply with the administration schedules.

In one embodiment, the method includes administering to the human at most three doses of the identical immunogenic composition. For example, in a preferred embodiment, the method does not include administering to the human a first dose of a first composition, administering to the human a second dose of a second composition, and administering to the human a third dose of a third composition, wherein the first, second, and third compositions are not identical. In another embodiment, the method includes administering to the human at most four doses of the identical immunogenic composition.

### EXAMPLES

The following Examples illustrate embodiments of the invention. Unless noted otherwise herein, reference is made in the following Examples to a vaccine candidate or immunogenic composition including a mixture of genetically modified C. difficile toxoid A, i.e., polypeptide, (comprising SEQ ID NO: 4, wherein the initial methionine is not present) and genetically modified C. difficile toxoid B, i.e., polypeptide, (comprising SEQ ID NO: 6, wherein the initial methionine is not present) that were further chemically inactivated by 1-ethyl-3-(3-dimethylaminopropyl) carbodiimide) (EDC) and N-Hydroxysuccinimide (NHS) to eliminate residual cytotoxicity but retain native antigenic structure and generate a neutralizing antibody response, as described in Example 21 of WIPO Patent Application WO/2012/143902, U.S. Pat. No. 9,187,536, and WIPO Patent Application WO/2014/060898, WIPO Patent Application WO/2012/143902, U.S. Pat. No. 9,187,536, and WIPO Patent Application WO/2014/060898, which are each incorporated by reference herein in their respective entireties. Briefly, after purification, the genetic mutant toxins (SEQ ID NO: 4 and SEQ ID NO: 6) are inactivated for 2 hours at 25° C. using 0.5 mg EDC and 0.5 mg NHS per mg of purified genetic mutant toxin A and B (approximately 2.6 mM and 4.4 mM respectively). The reaction is quenched by the addition of glycine to a final concentration of 100 mM and the reactions incubate for an additional 2 hours at 25° C. The inactivation is carried out at pH 7.0±0.5 in 10 mM phosphate, 150 mM sodium chloride buffer. The inactivation period is set to exceed three times the period needed for reduction in the EC50 in IMR90 cells to greater than 1000 ug/mL. After 2 hours, the biological activity is reduced 7 to 8 log₁₀ relative to the native toxin. Following the 4 hour incubation, the inactivated mutant toxin is exchanged into the final drug substance buffer by diafiltration. For example, using a 100 kD regenerated cellulose acetate ultrafiltration cassette, the inactivated toxin is concentrated to 1-2 mg/mL and buffer-exchanged. More specifically, the vaccine composition includes (a) a first polypeptide, which includes the amino acid sequence set forth in SEQ ID NO: 4, wherein the methionine residue at position 1 of SEQ ID NO: 4 is not present, wherein a side chain of a lysine residue of the first polypeptide is crosslinked to a beta-alanine moiety, and wherein the first polypeptide further includes a crosslink between a side chain of an aspartic acid residue of the first polypeptide and a glycine moiety, and a crosslink between a side chain of a glutamic acid residue of the first polypeptide and a glycine moiety; and (b) a second polypeptide, which includes the amino acid sequence set forth in SEQ ID NO: 6, wherein the methionine residue at position 1 of SEQ ID NO: 6 is not present, wherein a side chain of a lysine residue of the second polypeptide is crosslinked to a beta-alanine moiety, and wherein the second polypeptide further includes a crosslink between a side chain of an aspartic acid residue of the second polypeptide and a glycine moiety, and a crosslink between a side chain of a glutamic acid residue of the second polypeptide and a glycine moiety.

The investigational C. difficile vaccine is composed of 2 toxoids (A and B) in equal amounts. The vaccine was provided as a sterile lyophilized powder at dosage strengths of 100 μg and 200 μg of toxoids A and B combined per dose. The vaccine was prepared for injection by resuspending the lyophilized vaccine with the aluminum hydroxide diluent immediately before use. The aluminum hydroxide diluent was supplied as a 1-mg aluminum/mL (as aluminum hydroxide) liquid suspension.

In preclinical experiments, the vaccine candidate was studied either alone or in combination with an adjuvant. In the hamster model, all vaccine formulations demonstrated a survival benefit, providing at least 90% protection from a lethal challenge with C. difficile spores in the immunized hamsters. In nonhuman primates, all of the toxoid vaccine formulations tested induced robust neutralizing anti-toxin antibody responses to both C. difficile toxin A and C. difficile toxin B.

### Example 1

### Phase 1, First in Human Study, 3-Dose Regimen at Months 0, 1, and 6 (B5091001)

The exemplary C. difficile vaccine candidate was assessed in the first-in-human (FIH) B5091001 Phase 1 study conducted in the USA, in which 192 healthy adults aged 50 to 85 years were enrolled. This was a dose-escalation, placebo-controlled, randomized, observer-blinded study to evaluate the safety, tolerability, and immunogenicity of C. difficile vaccine, administered as a 3-dose regimen at Months 0, 1, and 6. Three (3) antigen dose levels (50, 100, and 200 μg) of the vaccine candidate were assessed either alone or in combination with aluminum hydroxide.

The analysis of safety demonstrated that both formulations and all 3 dose levels were generally well tolerated. Local reactions were predominantly mild or moderate and comprised mostly of injection site pain. No actual severe or Grade 4 local reactions were reported. In the 65- to 85-year age cohort, at the 200-μg dose level, local reactions tended to occur more frequently in the toxoid-alone compared to the aluminum hydroxide-containing dose groups. After all 3 doses, the frequency and severity of local reactions did not increase with increasing dose level or number of doses for any of the dose groups. Systemic events were predominantly mild to moderate and comprised mostly headache and fatigue. There was no evidence of increased frequency of systemic events with increasing dose level or number of doses for any of the dose groups.

The analysis of immunogenicity demonstrated a limited antibody response after Dose 1, but after Dose 2 there were marked increases in antibody titers against both toxin A and toxin B, which were generally maximal 7 days after Dose 2 and stable 1 month after Dose 2. Seven (7) days after Dose 3, a substantial booster response was evident, which was slightly more marked again 1 month after Dose 3. Overall, robust anti-toxin neutralizing responses were elicited by both formulations, although there was a trend for greater responses in recipients of the toxoid-alone formulation. For example, 1 month after Dose 3 in the 65- to 85-year age cohort, the geometric mean fold rises (GMFRs) from baseline (before Dose 1) in toxin A-specific neutralizing antibody titers ranged from 131 to 254 in the toxoid-alone dose groups and from 42 to 80 in the aluminum hydroxide-containing dose groups. The corresponding ranges for toxin B-specific neutralizing antibody titers were from 2953 to 4922 and from 136 to 484 respectively.

Preclinical data generated in rhesus macaques support the use of a 3-dose regimen of C. difficile vaccine, administered with or without aluminum hydroxide at Weeks 0, 2, and 4. Furthermore, in a rabbit toxicology study, a 4-dose regimen of C. difficile vaccine (up to 400-μg dose levels) given on Days 1, 8, 22, and 36 did not demonstrate adverse toxicological findings, and resulted in an increase in antitoxin A- and antitoxin B-neutralizing antibody titers, confirming the anticipated immunologic response by the animals to the administered immunogen.

These preclinical and toxicology data, as well as the encouraging immune response observed after 3 doses in the B5091001 Phase 1 study, supported the Phase 2 Study B5091003.

### Example 2

### Phase 2 Study, 3-Dose Regimen at Days 1, 8, and 30 (B5091003)

The Phase 2 Study B5091003 was designed to evaluate the safety, tolerability, and immunogenicity of the 100- and 200-μg antigen dose levels (total for toxoids A and B) of a toxoid-alone C. difficile vaccine in a 3-dose regimen administered at Days 1, 8, and 30 in healthy adults 50 to 85 years of age.

Vaccinations in Study B5091003 were stopped following the occurrence of injection site erythema. There were no accompanying severe systemic symptoms in the subjects, there was no report that the redness impacted their daily activities, and all local reactions fully resolved. The local reactogenicity observed with the toxoid-alone formulation may have been due to free toxoid interacting with the elicited immune response.

### Example 3

### Phase 2 Study, 3-Dose Regimen at Days 1, 8, and 30 (Day Regimen) or Months 0, 1, and 6 (Month Regimen) (B5091009)

Adsorption onto aluminum has been shown to bind and slowly release the vaccine constituents from the injection site. In addition, there is a potential need for rapid induction of immune response and prolonged duration of protection against CDI. Accordingly, study B5091009 evaluated the safety, tolerability, and immunogenicity of 2 antigen dose levels of the aluminum hydroxide-containing vaccine (i.e., 100 μg and 200 μg total toxoids) selected from the FIH B5091001 study in 2 different dosing regimens: Days 1, 8, and 30 or Months 0, 1, and 6.

The primary immunogenicity endpoint was assessed based upon the ability of the vaccine to induce toxin A- and toxin B-specific neutralizing antibody levels greater than or equal to a specified threshold estimate for each C. difficile vaccine toxoid. These specified thresholds were derived from a Phase 2 efficacy study demonstrating that passive administration of 2 mAbs against toxin A and toxin B were associated with protection against CDI. In addition to showing efficacy of anti-toxin mAbs against recurrent CDI, the Phase 2 efficacy study also suggested that anti-toxin A- and anti-toxin B-neutralizing mAb levels above a threshold of 10 μg/mL (“protective” threshold level) were associated with protection against CDI recurrence.

To translate the toxin A and toxin B “protective” threshold from the Phase 2 efficacy study into 50% neutralization titers elicited by the vaccine candidate, advantage was taken of the observations that (1) the same cytotoxicity assay was used to measure toxin neutralization and (2) the inhibitory mAb concentration that neutralizes 50% of the toxins (IC₅₀ [50% inhibitory concentration]) had been published (IC₅₀ values for the toxin A and toxin B mAbs are 100 ng/mL and 15 ng/mL, respectively). The “protective” 50% neutralization titer for each anti-toxin antibody is, therefore, calculated to be the antibody concentration at the protective threshold of 10 μg/mL divided by the respective mAb IC₅₀.

To qualify the 50% neutralization titer assay for clinical use, provide further robustness to the assay, and assure consistent long-term performance in clinical development, a reference standard and appropriate controls were added to the assay, thereby permitting the read out of a neutralization titer as neutralization units/mL defined by the reference standard. Prior to analysis of the current study, serum samples from vaccinated humans were used to demonstrate a linear relationship between 50% neutralization titers and neutralization units/mL when performing the neutralization assay. Based on these correlation studies, “protective” neutralization threshold values were calculated and used to analyze the clinical data in this study.

For the immunogenicity analyses, the “protective” thresholds for antitoxin A- and toxin B-neutralizing antibody responses were 219 and 2586 neutralization units/mL, respectively.

Several of the immunogenicity endpoints for Study B5091009 were assessed based upon these “protective” thresholds.

As used herein, unless expressly defined otherwise, the “specified threshold” value is defined as 219 neutralization units/mL for toxin A and 2586 neutralization units/mL for toxin B.

Since it will be important to provide vaccinated subjects with prolonged protection against CDI, and since individuals targeted for vaccination may have diminished capacity to mount and maintain an immune response, subjects in the present study were monitored after their third vaccination to assess antibody persistence and response to a fourth vaccination. Therefore, subjects in both dosing regimens who received the first 3 doses of C. difficile vaccine (100 μg or 200 μg) were asked to enter an extension stage and were rerandomized in a 1:1 ratio to receive C. difficile vaccine or placebo. These subjects will receive a fourth dose of either C. difficile vaccine at the same antigen dose level (100 μg or 200 μg) as they received previously or placebo, approximately 1 year after their third dose. These subjects will be followed to assess antibody persistence. Subjects originally randomized to placebo in either dosing regimen will not be continued into the extension stage.

The analyses for this clinical study were performed when all subjects had completed Visit 9 (Month 13 for subjects on the Day 1, 8, and 30 regimen [day regimen] and Month 18 for subjects on the Month 0, 1, and 6 regimen [month regimen]) and all immunogenicity and safety data up to and including Visit 9 were available.

Methods of Analysis. For any C. difficile toxin A- or toxin B-specific neutralizing antibody level that was below the lower limit of quantitation (LLOQ), the LOD, defined as 0.5×LLOQ, was assigned. The LLOQs for the toxin A- and toxin B-specific neutralization assays were 158.0 neutralization units/mL and 249.5 neutralization units/mL, respectively. No other missing assay data were imputed in the analyses. All immunogenicity analyses were performed after the imputation of the antibody levels that were below the LLOQ. If the toxin A-specific neutralizing antibody level was LLOQ for toxin-A, the subject was considered seropositive for toxin A. If the toxin B-specific neutralizing antibody level was LLOQ for toxin B, the subject was considered seropositive for toxin B. Conversely, if an antibody level was <LLOQ, the subject was considered seronegative. The immunogenicity data were summarized according to the vaccine dose as randomized. For the original planned stage, all immunogenicity data were summarized separately for each assigned dosing regimen (Days 1, 8, and 30 and Months 0, 1, and 6). Within each assigned dosing regimen, there were 3 vaccine groups (100 μg C. difficile, 200 μg C. difficile, and placebo).

### Example 4

### Phase 2 Study, 3-Dose Regimen at Days 1, 8, and 30 (Day Regimen) or Months 0, 1, and 6 (Month Regimen) (B5091009)—Overall Study Design and Plan

Approximately 854 healthy adults, aged 65 to 85 years, were planned to be enrolled at approximately 15 sites in the USA. Subjects were assigned to 1 of the 2 dosing regimens and then randomly assigned in parallel in a 3:3:1 ratio to receive C. difficile vaccine (100 μg or 200 μg) or placebo (saline) (Table 3).

TABLE 3
Vaccine Groups and Planned Number of Subjects
per Group and per Dose Regimen

| Vaccine | | Dosing | Number of |
|---|---|---|---|
| Group | Vaccine Formulation Description | Regimen | Subjects |
| 1^{a} | Aluminum hydroxide-containing | Days 1, 8, | 183 |
| | C. difficile vaccine (100-μg | and 30 | |
| | antigen dose) | | |
| 2^{a} | Aluminum hydroxide-containing | Days 1, 8, | 183 |
| | C. difficile vaccine (200-μg | and 30 | |
| | antigen dose) | | |
| 3 | Placebo (saline) | Days 1, 8, | 61 |
| | | and 30 | |
| 4^{a} | Aluminum hydroxide-containing | Months 0, 1, | 183 |
| | C. difficile vaccine (100-μg | and 6 | |
| | antigen dose) | | |
| 5^{a} | Aluminum hydroxide-containing | Months 0, 1, | 183 |
| | C. difficile vaccine (200-μg | and 6 | |
| | antigen dose) | | |
| 6 | Placebo (saline) | Months 0, 1, | 61 |
| | | and 6 | |
| Total | | | 854 |

^{a}Subjects in these groups were asked to enter the extension stage.
Source: Statistical analysis plan (Version 2.0), Table 4.

This was a Phase 2, placebo-controlled, randomized, observer-blinded study to assess the safety, tolerability, and immunogenicity of 2 antigen dose levels (100 μg and 200 μg total toxoid) of aluminum hydroxide-containing C. difficile vaccine administered as a 3-dose regimen either at Days 1, 8, and 30 or Months 0, 1, and 6 in healthy adults aged 65 to 85 years. The 100-μg antigen dose level (total for toxoids A and B) and the 200-μg antigen dose level (total for toxoids A and B) were chosen for this study because the immunogenicity and safety results from the FIH study (B5091001) showed that antigen dose levels of 100 and 200 μg induced similar immune responses. For the control group, the placebo consisted of a sterile normal saline solution for injection (0.9% sodium chloride) in a 0.5-mL dose. Aluminum hydroxide was chosen as a diluent as adsorption onto aluminum has been shown to bind and slowly release the vaccine constituents from the injection site.

The study was placebo controlled (although randomization in the original planned stage was weighted towards the active formulations) to provide a comparative assessment of the safety and tolerability of the investigational vaccine formulation, as well as to control for any potential change over time in the natural background titers of antibodies to C. difficile toxins A and B.

Subjects on the day regimen received 1 dose of C. difficile vaccine/placebo at Visits 1 (Day 1), 2 (Day 8), and 4 (Day 30). Subjects on the month regimen received 1 dose of C. difficile vaccine/placebo at Visits 1 (Day 1), 2 (Day 30), and 5 (Month 6).

Primary Immunogenicity Endpoints—At Day 37 (7 days after Dose 3 for subjects on the day regimen) and at Month 7 (1 month after Dose 3 for subjects on the month regimen), the proportions of subjects in each vaccine group with: Toxin A-specific neutralizing antibody level (neutralization units/mL) the specified threshold for toxin A; Toxin B-specific neutralizing antibody level (neutralization units/mL) the specified threshold for toxin B; and both toxin A- and toxin B-specific neutralizing antibody levels (neutralization units/mL) the specified threshold for toxin A and the specified threshold for toxin B, respectively. The thresholds were defined as 219 neutralization units/mL for toxin A and 2586 neutralization units/mL for toxin B.

Secondary Immunogenicity Endpoints—At Day 37 (7 days after Dose 3 for subjects on the day regimen) and at Month 7 (1 month after Dose 3 for subjects on the month regimen): Toxin A- and toxin B-specific neutralizing antibody levels, expressed as geometric mean concentrations (GMCs) (neutralization units/mL). GMFRs from baseline (before Dose 1) in: Toxin A-specific; and Toxin B-specific neutralizing antibody levels (neutralization units/mL). Proportions of subjects in each vaccine group with ≥4-fold, ≥8-fold, ≥16-fold, and <32-fold rises from baseline in: Toxin A-specific; Toxin B-specific; and both toxin A- and toxin B-specific neutralizing antibody levels (neutralization units/mL). For subjects on the day regimen, on Day 1 (immediately before Dose 1), Day 8 (immediately before Dose 2), Day 15 (7 days after Dose 2), Day 30 (immediately before Dose 3), Month 2 (1 month after Dose 3), Month 4 (3 months after Dose 3), Month 7 (6 months after Dose 3), and Month 13 (12 months after Dose 3); or for subjects on the month regimen, on Day 1 (immediately before Dose 1), Day 30 (immediately before Dose 2), Day 37 (7 days after Dose 2), Month 2 (1 month after Dose 2), Month 6 (immediately before Dose 3), Day 187 (7 days after Dose 3), Month 12 (6 months after Dose 3), and Month 18 (12 months after Dose 3): Proportions of subjects in each vaccine group with: Toxin A-specific neutralizing antibody level (neutralization units/mL)≥the specified threshold for toxin A; Toxin B-specific neutralizing antibody level (neutralization units/mL)≥the specified threshold for toxin B; and Both toxin A- and toxin B-specific neutralizing antibody levels (neutralization units/mL)≥the specified threshold for toxin A and the specified threshold for toxin B, respectively (these parameters will also be assessed at baseline). The thresholds were defined as 219 neutralization units/mL for toxin A and 2586 neutralization units/mL for toxin B. Toxin A- and toxin B-specific neutralizing antibody levels, expressed as GMCs (neutralization units/mL). GMFRs from baseline in: Toxin A-specific; and Toxin B-specific neutralizing antibody levels (neutralization units/mL). Proportions of subjects in each vaccine group with ≥4-fold, ≥8-fold, ≥16-fold, and ≥32-fold rises from baseline in: Toxin A-specific; Toxin B-specific; and Both toxin A- and toxin B-specific neutralizing antibody levels (neutralization units/mL). Baseline in the above endpoints was the associated last measurement prior to the first vaccination on Day 1.

Immunogenicity analyses. The analyses here were performed when all subjects had completed Visit 9 (Month 13 for subjects on the day regimen and Month 18 for subjects on the month regimen) and all immunogenicity and safety data up to and including Visit 9 were available. In this section, immunogenicity results are presented separately for the month regimen and the day regimen.

The primary immunogenicity endpoints were the proportions of subjects in each vaccine group with toxin A-, toxin B-, and both toxin A- and toxin B-specific neutralizing antibody titers≥specified thresholds (219 neutralization units/mL for toxin A-specific antibody and 2586 neutralization units/mL for toxin B-specific antibody) at Day 37 (7 days after Dose 3 for subjects on the day regimen) and at Month 7 (1 month after Dose 3 for subjects on the month regimen). The secondary immunogenicity endpoints were as follows: Toxin A- and toxin B-specific neutralizing antibody levels at all sampling time points, expressed as GMCs. Toxin A- and toxin B-specific neutralizing antibody levels at all sampling time points, expressed as GMFRs. The proportions of subjects in each vaccine group achieving defined fold rises from baseline in toxin A-, toxin B-, and both toxin A- and toxin B-specific neutralizing antibody levels at all sampling time points.

Immunogenicity Conclusions. Compared with the day regimen, the month regimen achieved a higher immune response at Day 187 and Month 7 for both toxin A and toxin B. Compared with the month regimen, the day regimen achieved a higher earlier immune response at Day 37 and Month 2 for toxin A, but responses were similar at these time points for toxin B. The 200-μg dose level was more immunogenic than the 100-μg dose level in both regimens. For toxin A, baseline seropositivity may have enhanced the magnitude of the immune response soon after Dose 1 (particularly for the 100-μg dose level), but, in both regimens, there was little difference in the immune response after Dose 3 compared with the response for subjects who were seronegative for toxin A at baseline. For toxin B, baseline seropositivity enhanced the magnitude of the immune response in both regimens. The proportions of subjects achieving both toxin A- and toxin B-specific neutralizing antibody levels threshold, at Month 7 in the month regimen or Day 37 in the day regimen, were similar when stratified by age quintile from 65 to 85 years (although the number of subjects 80 to 85 years of age was small).

This was a Phase 2, placebo-controlled, randomized, observer-blinded study to assess the safety, tolerability, and immunogenicity of 2 antigen dose levels (100 μg and 200 μg total toxoid) of aluminum hydroxide-containing C. difficile vaccine administered as a 3-dose regimen: either at Days 1, 8, and 30 (day regimen) or Months 0, 1, and 6 (month regimen). Overall, the C. difficile vaccine was highly immunogenic, well tolerated, and exhibited an acceptable safety profile.

Immunogenicity Discussion. The primary immunogenicity objectives of this study was to describe the immunogenicity of 2 antigen dose levels (100 μg and 200 μg total toxoid) of C. difficile vaccine when administered as a 3-dose regimen (either Days 1, 8, and 30 or Months 0, 1, and 6) to healthy adults aged 65 to 85 years, as measured by C. difficile toxin A- and toxin B-specific neutralizing antibody levels at Day 37 (7 days after Dose 3) for the day regimen and as measured by C. difficile toxin A- and toxin B-specific neutralizing antibody levels at Month 7 (1 month after Dose 3) for the month regimen. Immunogenicity was assessed through measurement of toxin A- and toxin B-specific neutralizing antibody titers throughout the course of the study. Results were expressed as GMCs, proportions of subjects achieving various fold rises, and proportions of subjects achieving titers above the specified threshold level for each toxin.

In both the month regimen and in the day regimen the 100-μg and 200-μg C. difficile groups were immunogenic when compared with placebo. The 200-μg dose level was more immunogenic than the 100-μg dose level based on the magnitude and durability of response.

The proportions of subjects in the month regimen achieving titers above the specified threshold level for each toxin were highest at Day 187 (1 week after Dose 3) and Month 7 (1 month after Dose 3) for both toxin A and toxin B.

The proportions of subjects in the day regimen achieving titers above the specified threshold level for toxin A were higher at Day 37 and Month 2 for toxin A, but proportions were similar at these time points for toxin B.

Prior to vaccination, the majority of subjects in both dosing regimens were seronegative for toxin A and for toxin B. In the month regimen, 69.8% of subjects were seronegative, 8.7% of subjects were seropositive for toxin A only, 19.0% of subjects were seropositive for toxin B only, and 2.6% of subjects were seropositive for both toxin A and toxin B. In the day regimen, 75.4% of subjects were seronegative, 4.9% of subjects were seropositive for toxin A only, 16.4% of subjects were seropositive for toxin B only, and 3.0% of subjects were seropositive for both toxin A and toxin B.

For toxin A, baseline seropositivity may have enhanced the magnitude of the immune response soon after Dose 1 (particularly for the 100-μg dose level), but, in both regimens, there was little difference in the immune response after Dose 3 compared with the response for subjects who were seronegative for toxin A at baseline.

For toxin B, baseline seropositivity enhanced the magnitude of the immune response in both regimens. For subjects who were seronegative for toxin B at baseline, only the third dose given at Month 6 resulted in a substantial proportion of subjects achieving the specified threshold.

The proportions of subjects achieving both toxin A- and toxin B-specific neutralizing antibody levels≥threshold, at Month 7 in the month regimen or Day 37 in the day regimen, were similar when stratified by age quintile from 65 to 85 years (although the number of subjects 80 to 85 years of age was small).

The 200-μg dose level was numerically more immunogenic than the 100-μg dose level in both dosing regimens.

The month regimen resulted in a higher post-Dose 3 response for both the 100-μg and 200-μg dose levels, particularly for toxin B in subjects who were seronegative at baseline.

The proportions of subjects achieving both toxin A- and toxin B-specific neutralizing antibody levels≥threshold were similar when stratified by age quintile from 65 to 85 years (although the number of subjects 80 to 85 years of age was small).

### Example 5

### Phase 2 Study, 3-Dose Regimen at Days 1, 8, and 30 (Day Regimen) or Months 0, 1, and 6 (Month Regimen) (B5091009)-Immunogenicity Evaluation, Month Regimen

Month regimen. The proportions of subjects achieving toxin A-, toxin B-, and both toxin A- and toxin B-specific neutralizing antibody titers≥the specified thresholds by subjects' baseline serostatus were assessed for the month regimen. All (100.0%) randomized subjects were evaluated for baseline serostatus. The majority were baseline seronegative for both toxin A and toxin B. Among the baseline seronegative subjects, the proportion achieving toxin A-specific neutralizing antibody titers≥the specified threshold reached 98.0% at Month 7 in the 100-μg C. difficile group and 95.0% at Month 7 in the 200-μg C. difficile group. The proportion achieving toxin B-specific neutralizing antibody titers≥the specified threshold reached 69.4% at Month 7 in the 100-μg C. difficile group and 85.0% at Month 7 in the 200-μg C. difficile group. For baseline seropositive subjects, the proportions achieving toxin A-specific neutralizing antibody titers≥the specified threshold reached 100.0% at Month 7 in the 100-μg C. difficile group and 100.0% at Month 7 in the 200-μg C. difficile group. The proportions achieving toxin B-specific neutralizing antibody titers≥the specified threshold reached 100.0% at Day 187 and Month 7 in the 100-μg C. difficile group and 96.8% at Day 37, Month 2, and Month 7 in the 200-μg C. difficile group.

For toxin A, at Month 7 in the month regimen, 160 subjects (95% CI: 94.7%; 99.6%) in the 100-μg C. difficile group and 151 subjects (95% CI: 91.1%; 98.2%) in the 200-μg C. difficile group achieved the specified threshold, compared with 1 subject (95% CI: 0.0%; 10.1%) in the placebo group.

For toxin B, at Month 7 in the month regimen, 122 (74.8%) subjects (95% CI: 67.5%; 81.3%) in the 100-μg C. difficile group and 138 (87.3%) subjects (95% CI: 81.1%; 92.1%) in the 200-μg C. difficile group achieved the specified threshold, compared with 4 (7.5%) subjects (95% CI: 2.1%; 18.2%) in the placebo group.

For both toxin A and toxin B, at Month 7 in the month regimen, 121 (74.2%) subjects (95% CI: 66.8%; 80.8%) in the 100-μg C. difficile group and 136 (86.1%) subjects (95% CI: 79.7%; 91.1%) in the 200-μg C. difficile group achieved the specified threshold, compared with no subjects (95% CI: 0.0%; 6.7%) in the placebo group

In the month regimen, 1 subject in the 100-μg C. difficile group, 2 (1.3%) subjects in the 200-μg C. difficile group, and 1 subject in the placebo group had toxin A-specific neutralizing antibody levels the specified threshold at baseline. After Doses 1 and 2 but prior to Dose 3, the proportions of subjects achieving the toxin A-specific threshold value were limited, with 71 (43.6%) subjects (95% CI: 35.8%; 51.5%) in the 100-μg C. difficile group and 89 (56.7%) subjects (95% CI: 48.6%; 64.6%) in the 200-μg C. difficile group at Day 37. After Dose 3, the proportions of subjects achieving this threshold value increased at Day 187. For the 100-μg C. difficile group at Month 7, 160 (98.2%) subjects achieved the toxin A-specific threshold value and the proportion of subjects achieving the threshold value decreased to 75 (48.7%) subjects at Month 18. For the 200-μg C. difficile group at Month 7, 151 (95.6%) subjects achieved the toxin A-specific threshold value and the proportion of subjects achieving this threshold value decreased to 81 (53.3%) subjects at Month 18.

In the month regimen, 4 (2.5%) subjects in the 100-μg C. difficile group, 7 (4.4%) subjects in the 200-μg C. difficile group, and 2 (3.8%) subjects in the placebo group had toxin B-specific neutralizing antibody levels the specified threshold at baseline. After Doses 1 and 2 but prior to Dose 3, 47 (28.8%) subjects (95% CI: 22.0%; 36.4%) in the 100-μg C. difficile group achieved the threshold value at Day 37 and at Month 2, and 60 (38.2%) subjects (95% CI: 30.6%; 46.3%) in the 200-μg C. difficile group achieved the threshold value at Day 37. After Dose 3, the proportions of subjects achieving this threshold value increased at Day 187. For the 100-μg C. difficile group at Month 7, 122 (74.8%) subjects achieved the toxin B-specific threshold value and the proportion of subjects achieving the threshold value decreased to 53 (34.4%) subjects at Month 18. For the 200-μg C. difficile group at Month 7, 138 (87.3%) subjects achieved the toxin B-specific threshold value and the proportion of subjects achieving this threshold value decreased to 72 (47.4%) subjects at Month 18.

Overall, results for the proportions of subjects achieving both toxin A- and toxin B-specific neutralizing antibody levels the specified thresholds were similar to those of toxin B.

Toxin A- and toxin B-specific neutralizing antibody Geometric Mean Concentrations (GMCs) for each time point were assessed. For the month regimen, at baseline, the toxin A-specific neutralizing antibody GMC was below the LLOQ (158.0 neutralization units/mL) for subjects in the 100-μg C. difficile, 200-μg C. difficile, and placebo groups. Compared to baseline, for subjects in the 100-μg C. difficile group, an increase in GMCs was observed at Day 30 (137 neutralization units/mL), was maximal at Month 7 (1245 neutralization units/mL), and decreased to 214 neutralization units/mL at Month 18. Compared to baseline, for subjects in the 200-μg C. difficile group, an increase in GMCs was observed at Day 30 (149 neutralization units/mL), was maximal at Month 7 (1380 neutralization units/mL), and decreased to 257 neutralization units/mL at Month 18. The toxin A-specific neutralizing antibody GMCs for the placebo group were 93 neutralization units/mL at all time points.

For the month regimen, at baseline, the toxin B-specific neutralizing antibody GMC was below the LLOQ (249.5 neutralization units/mL) for subjects in the 100-μg C. difficile, 200-μg C. difficile, and placebo groups. Compared to baseline, for subjects in the 100-μg C. difficile group, an increase in GMCs was observed at Day 30 (570 neutralization units/mL), was maximal at Month 7 (6255 neutralization units/mL), and decreased to 1248 neutralization units/mL at Month 18. Compared to baseline, for subjects in the 200-μg C. difficile group, an increase in GMCs was observed at Day 30 (909 neutralization units/mL), was maximal at Month 7 (9549 neutralization units/mL), and decreased to 2178 neutralization units/mL at Month 18. The toxin A-specific neutralizing antibody GMCs for the placebo group were 263 neutralization units/mL at all time points.

For the month regimen, at all of the postbaseline visits, toxin A-specific neutralizing antibody GMCs were higher in the 200-μg C. difficile group compared with the 100-μg C. difficile group. Both the 100-μg and 200-μg C. difficile groups had higher postbaseline GMCs than the placebo group; however, no clear dose response was evident between the 100-μg and 200-μg C. difficile groups. For the month regimen, at all of the postbaseline visits, toxin B-specific neutralizing antibody GMCs were higher in the 200-μg C. difficile group compared with the 100-μg C. difficile group. Both the 100-μg and 200-μg C. difficile groups had higher postbaseline GMCs than the placebo group. A clear dose response was evident between the 100-μg and 200-μg C. difficile groups after Dose 3 (Month 12 and Month 18). Toxin A- and toxin B-specific neutralizing antibody GMCs by subjects' baseline serostatus was summarized for the month regimen. Toxin A- and toxin B-specific neutralizing antibody GMCs by subjects' age and baseline serostatus was summarized for the day regimen.

For the month regimen, toxin A- and toxin B-specific neutralizing antibody Geometric Mean Fold Rise (GMFRs) from baseline were calculated at Day 30, Day 37, Month 2, Month 6, Day 187, Month 7, Month 12, and Month 18.

After Dose 2, an increase in toxin A-specific neutralizing antibody GMFRs was observed at Day 37 (100-μg C. difficile group, 2.70; 200-μg C. difficile group, 3.78). After Dose 3, a booster response was evident at Day 187 (100-μg C. difficile group, 5.85; 200-μg C. difficile group, 8.54). This booster response was maximal at Month 7 (1 month after Dose 3), with GMFRs of 14.58 for the 100-μg C. difficile group and 15.85 for the 200-μg C. difficile group. The GMFRs decreased to 2.51 for the 100-μg C. difficile group and to 2.95 for the 200-μg C. difficile group at Month 18.

After Dose 2, an increase in toxin B-specific neutralizing antibody GMFRs was observed at Day 37 (100-μg C. difficile group, 3.75; 200-μg C. difficile group, 5.59). After Dose 3, a substantial booster response was evident at Day 187 (100-μg C. difficile group, 15.85; 200-μg C. difficile group, 24.44). This booster response was maximal at Month 7 (1 month after Dose 3), with GMFRs of 35.43 for the 100-μg C. difficile group and 49.98 for the 200-μg C. difficile group. The GMFRs decreased to 7.07 for the 100-μg C. difficile group and to 11.21 for the 200-μg C. difficile group at Month 18.

Between Day 30 and Month 18, toxin A- and toxin B-specific neutralizing antibody GMFRs were higher for the 200-μg C. difficile group compared with the 100-μg C. difficile group.

Toxin A- and toxin B-specific neutralizing antibody GMFRs by subjects' baseline serostatus for each time point were assessed for the month regimen. Among the baseline seronegative subjects, an increase in toxin A-specific neutralizing antibody GMFRs was observed at Day 37 (100-μg C. difficile group, 2.74; 200-μg C. difficile group, 4.07). After Dose 3, a booster response was evident at Day 187 (100-μg C. difficile group, 6.29; 200-μg C. difficile group, 9.37). This booster response was maximal at Month 7, with GMFRs of 15.88 for the 100-μg C. difficile group and 17.32 for the 200-μg C. difficile group. The GMFRs decreased to 2.59 for the 100-μg C. difficile group and to 3.12 for the 200-μg C. difficile group at Month 18. After Dose 2, toxin B-specific neutralizing antibody GMFR leveled off for the 100-μg C. difficile (Day 30: 2.45; Day 37: 2.67; Month 2: 2.79) and 200-μg C. difficile groups (Day 30: 3.40; Day 37: 3.95; Month 2: 3.94). After Dose 3, a substantial booster response was evident at Day 187 (100-μg C. difficile group, 16.00; 200-μg C. difficile group, 26.67). This booster response was maximal at Month 7, with GMFRs of 39.29 for the 100-μg C. difficile group and 61.04 for the 200-μg C. difficile group. The GMFRs decreased to 6.95 for the 100-μg C. difficile group and to 11.57 for the 200-μg C. difficile group at Month 18.

Among the baseline seropositive subjects, an increase in toxin A-specific neutralizing antibody GMFRs was observed at Day 37 (100-μg C. difficile group, 2.37; 200-μg C. difficile group, 2.13). After Dose 3, a booster response was evident at Day 187 (100-μg C. difficile group, 3.03; 200-μg C. difficile group, 4.18). This booster response was maximal at Month 7, with GMFRs of 6.65 for the 100-μg C. difficile group and 7.96 for the 200-μg C. difficile group. The GMFRs decreased to 1.87 for the 100-μg C. difficile group and to 1.91 for the 200-μg C. difficile group at Month 18. After Dose 2, an increase in toxin B-specific neutralizing antibody GMFRs was observed at Day 37 (100-μg C. difficile group, 17.97; 200-μg C. difficile group, 23.01). After Dose 3, a substantial booster response was evident at Month 7 (100-μg C. difficile group, 21.96; 200-μg C. difficile group, 21.99). This booster response was maximal at Month 7. The GMFRs decreased to 7.68 for the 100-μg C. difficile group and to 9.91 for the 200-μg C. difficile group at Month 18.

Between Day 30 and Month 18, toxin A-specific neutralizing antibody GMFRs were higher for the 200-μg C. difficile group compared with the 100-μg C. difficile group, except at Day 30 and Day 37 for the baseline seropositive subjects. After Day 187, toxin A-specific neutralizing antibody GMFRs were higher for the baseline seronegative subjects in both dose groups compared with the baseline seropositive subjects. Between Day 30 and Month 18, toxin B-specific neutralizing antibody GMFRs for baseline seronegative and seropositive subjects were higher for the 200-μg C. difficile group compared with the 100-μg C. difficile group. Between Day 30 and Month 6, toxin B-specific neutralizing antibody GMFRs were higher for the baseline seropositive subjects compared with the baseline seronegative subjects, but from Day 187 to Month 18, toxin B-specific neutralizing antibody GMFRs were higher for the baseline seronegative subjects compared with the baseline seropositive subjects.

The proportions of subjects achieving defined fold rises from baseline in toxin A-, toxin B-, and both toxin A- and toxin B-specific neutralizing antibody levels at Day 37 and at the other blood sampling time points were summarized.

Overall, greater proportions of subjects achieving defined fold rises from baseline toxin A-specific neutralizing antibody levels were observed for the 200-μg C. difficile group compared with the 100-μg C. difficile group. For the 100-μg C. difficile group, increases in proportions of subjects reaching a ≥4-fold rise were observed from Day 37, with 125 (76.7%) subjects reaching a ≥8-fold rise at Month 7. For the 100-μg C. difficile group, 77 (47.2%) subjects reached a ≥16-fold rise and 33 (20.2%) subjects reached a ≥32-fold rise in toxin A-specific neutralizing antibody levels at Month 7. For the 200-μg C. difficile group, increases in proportions of subjects reaching a ≥4-fold rise were observed from Day 37, with a marked increase in the proportion of subjects reaching a ≥8-fold rise at Day 187. For the 200-μg C. difficile group, 89 (56.3%) subjects reached a ≥16-fold rise and 34 (21.5%) subjects reached a ≥32-fold rise in toxin A-specific neutralizing antibody levels at Month 7.

Overall, greater proportions of subjects achieving defined fold rises from baseline toxin B-specific neutralizing antibody levels were observed for the 200-μg C. difficile group compared with the 100-μg C. difficile group. For the 100-μg C. difficile group, increases in proportions of subjects reaching a ≥4-fold rise were observed from Day 37, with 138 (84.7%) subjects reaching a ≥8-fold rise at Month 7. For the 100-μg C. difficile group, 85 (52.5%) subjects reached a ≥16-fold and 52 (32.1%) subjects reached a ≥32-fold rise in toxin B-specific neutralizing antibody levels at Day 187. For the 200-μg C. difficile group, increases in proportions of subjects reaching a ≥4-fold rise were observed from Day 187, with marked increases in the proportions of subjects reaching a ≥8-fold rise at Day 187. For the 200-μg C. difficile group, 105 (66.5%) subjects in the 200-μg C. difficile group reached a ≥32-fold rise in toxin B-specific neutralizing antibody levels at Month 7.

Overall, results for the proportions of subjects achieving defined fold rises from baseline for both toxin A- and toxin B-specific neutralizing antibody levels were similar to those of toxin A.

Reverse Cumulative Distribution Curves (RCDCs) displaying data for toxin A-specific neutralizing antibody levels measured at Day 1, Day 30, Day 37, Month 2, Month 6, Day 187, Month 7, Month 12, and Month 18 by vaccine group were assessed for the month regimen. RCDCs displaying data for toxin B-specific neutralizing antibody levels measured at Day 1, Day 30, Day 37, Month 2, Month 6, Day 187, Month 7, Month 12, and Month 18 by vaccine group were assessed for the month regimen. Overall, the observed curve shifted to the right for the vaccinated groups, indicating that immunization with the toxoid vaccine increased titer levels over that seen with placebo. This is consistent with the patterns shown in GMCs and the proportions of subjects achieving the specified thresholds.

### Example 6

### Phase 2 Study, 3-Dose Regimen at Days 1, 8, and 30 (Day Regimen) or Months 0, 1, and 6 (Month Regimen) (B5091009)-Immunogenicity Evaluation, Day Regimen

Day Regimen. The proportions of subjects achieving toxin A-, toxin B-, and both toxin A- and toxin B-specific neutralizing antibody titers≥the specified thresholds by subjects' baseline serostatus were assessed for the day regimen. The majority were baseline seronegative for both toxin A and toxin B. Among the baseline seronegative subjects, the proportion achieving toxin A-specific neutralizing antibody titers≥the specified threshold reached 67.7% at Day 37 in the 100-μg C. difficile group and 85.2% at Day 37 and Month 2 in the 200-μg C. difficile group. The proportion achieving toxin B-specific neutralizing antibody titers≥the specified threshold reached 14.0% at Day 15 and Day 37 in the 100-μg C. difficile group and 25.2% at Day 37 in the 200-μg C. difficile group. For baseline seropositive subjects, the proportions achieving toxin A-specific neutralizing antibody titers≥the specified threshold reached 80.0% at Day 37 in the 100-μg C. difficile group and 86.7% at Day 37 and Month 2 in the 200-μg C. difficile group. The proportions achieving toxin B-specific neutralizing antibody titers≥the specified threshold reached 97.1% at Day 30 in the 100-μg C. difficile group and 93.9% at Day 37 and Month 2 in the 200-μg C. difficile group.

For toxin A, at Day 37 in the day regimen, 117 (68.4%) subjects (95% CI: 60.9%; 75.3%) in the 100-μg C. difficile group and 141 (85.5%) subjects (95% CI: 79.1%; 90.5%) in the 200-μg C. difficile group achieved the specified threshold, compared with 7 (12.5%) subjects (95% CI: 5.2%; 24.1%) in the placebo group.

For toxin B, at Day 37 in the day regimen, 51 (29.8%) subjects (95% CI: 23.1%; 37.3%) in the 100-μg C. difficile group and 64 (38.8%) subjects (95% CI: 31.3%; 46.7%) in the 200-μg C. difficile group achieved the specified threshold, compared with 1 subject (95% CI: 0.0%; 9.6%) in the placebo group.

For both toxin A and toxin B, at Day 37 in the day regimen, 45 (26.3%) subjects (95% CI: 19.9%; 33.6%) in the 100-μg C. difficile group and 64 (38.8%) subjects (95% CI: 31.3%; 46.7%) in the 200-μg C. difficile group achieved the specified threshold, compared with no subjects (95% CI: 0.0%; 6.4%) in the placebo group.

In the day regimen, 2 (1.2%) subjects in the 100-μg C. difficile group and 1 subject in the 200-μg C. difficile group had toxin A-specific neutralizing antibody levels the specified threshold at baseline. After Doses 1 and 2 but prior to Dose 3, the proportions of subjects achieving the toxin A-specific threshold value were limited, with 29 (17.0%) subjects (95% CI: 11.7%; 23.4%) in the 100-μg C. difficile group and 41 (25.0%) subjects (95% CI: 18.6%; 32.3%) in the 200-μg C. difficile group at Day 15. After Dose 3, the proportions of subjects achieving this threshold value increased at Day 37. For the 100-μg C. difficile group at Day 37, 117 (68.4%) subjects achieved the toxin A-specific threshold value and the proportion of subjects achieving the threshold value decreased to 16 (10.1%) subjects at Month 13. For the 200-μg C. difficile group at Day 37 and at Month 2, 141 (85.5%) subjects achieved the toxin A-specific threshold value and the proportion of subjects achieving this threshold value decreased to 40 (25.8%) subjects at Month 13.

In the day regimen, 8 (4.7%) subjects in the 100-μg C. difficile group, 6 (3.7%) subjects in the 200-μg C. difficile group, and 3 (5.4%) subjects in the placebo group had toxin B-specific neutralizing antibody levels≥the specified threshold at baseline. After Doses 1 and 2 but prior to Dose 3, 52 (30.4%) subjects (95% CI: 23.6%; 37.9%) in the 100-μg C. difficile group and 61 (37.2%) subjects (95% CI: 29.8%; 45.1%) in the 200-μg C. difficile group achieved the threshold value at Day 15. After Dose 3, the proportions of subjects achieving this threshold value remained stable until Day 37. For the 100-μg C. difficile group at Day 30 and Day 37, 51 (29.8%) subjects achieved the toxin B-specific threshold value and the proportion of subjects achieving the threshold value decreased to 31 (19.6%) subjects at Month 13. For the 200-μg C. difficile group at Day 37, 64 (38.8%) subjects achieved the toxin B-specific threshold value and the proportion of subjects achieving this threshold value decreased to 48 (31.0%) subjects at Month 13.

Overall, results for the proportions of subjects achieving both toxin A- and toxin B-specific neutralizing antibody levels the specified thresholds were similar to those of toxin B.

Toxin A- and toxin B-specific neutralizing antibody GMCs for each time point were assessed. For the day regimen, at baseline, the toxin A-specific neutralizing antibody GMC was below the LLOQ (158.0 neutralization units/mL) for subjects in the 100-μg C. difficile, 200-μg C. difficile, and placebo groups. Compared to baseline, for subjects in the 100-μg C. difficile group, an increase in GMCs was observed at Day 15 (143 neutralization units/mL), was maximal at Day 37 (368 neutralization units/mL), and decreased to 105 neutralization units/mL at Month 13. Compared to baseline, for subjects in the 200-μg C. difficile group, an increase in GMCs was observed at Day 15 (192 neutralization units/mL), was maximal at Day 37 (556 neutralization units/mL), and decreased to 138 neutralization units/mL at Month 13. The toxin A-specific neutralizing antibody GMCs for the placebo group were 102 neutralization units/mL at all time points.

For the day regimen, at baseline, the toxin B-specific neutralizing antibody GMC was below the LLOQ (249.5 neutralization units/mL) for subjects in the 100-μg C. difficile, 200-μg C. difficile, and placebo groups. Compared to baseline, for subjects in the 100-μg C. difficile group, an increase in GMCs was observed at Day 8 (273 neutralization units/mL), was maximal at Day 15 (807 neutralization units/mL), and decreased to 447 neutralization units/mL at Month 13. Compared to baseline, for subjects in the 200-μg C. difficile group, an increase in GMCs was observed at Day 8 (290 neutralization units/mL), was maximal at Day 37 (1219 neutralization units/mL), and decreased to 828 neutralization units/mL at Month 13. The toxin A-specific neutralizing antibody GMCs for the placebo group were ≤211 neutralization units/mL at all time points.

For the day regimen, at all of the postbaseline visits, toxin A-specific neutralizing antibody GMCs were higher in the 200-μg C. difficile group compared with the 100-μg C. difficile group. Both the 100-μg and 200-μg C. difficile groups had higher postbaseline GMCs than the placebo group, and a clear dose response was evident between the 100-μg and 200-μg C. difficile groups.

For the day regimen, at all of the postbaseline visits, toxin B-specific neutralizing antibody GMCs were higher in the 200-μg C. difficile group compared with the 100-μg C. difficile group. Both the 100-μg and 200-μg C. difficile groups had higher postbaseline GMCs than the placebo group; however, no clear dose response was evident between the 100-μg and 200-μg C. difficile groups.

Toxin A- and toxin B-specific neutralizing antibody GMCs by subjects' baseline serostatus were summarized for the day regimen. Toxin A- and toxin B-specific neutralizing antibody GMCs by subjects' age and baseline serostatus were summarized for the day regimen.

For the day regimen, toxin A- and toxin B-specific neutralizing antibody Geometric Mean Fold Rise (GMFRs) from baseline were calculated at Day 8, Day 15, Day 30, Day 37, Month 2, Month 4, Month 7, and Month 13. After Dose 2, an increase in toxin A-specific neutralizing antibody GMFRs was observed at Day 15 (100-μg C. difficile group, 1.73; 200-μg C. difficile group, 2.27). After Dose 3, a booster response was evident at Day 37 (100-μg C. difficile group, 4.45; 200-μg C. difficile group, 6.56). This booster response was maximal at Day 37 (7 days after Dose 3) and was 3.56 for the 100-μg C. difficile group and 5.71 for the 200-μg C. difficile group at Month 2. The GMFRs decreased to 1.26 for the 100-μg C. difficile group and to 1.64 for the 200-μg C. difficile group at Month 13.

After Dose 2, a marked increase in toxin B-specific neutralizing antibody GMFRs was observed at Day 15 (100-μg C. difficile group, 4.31; 200-μg C. difficile group, 5.86). After Dose 3, a booster response was evident at Day 37 for the 200-μg C. difficile group (GMFR of 6.57), but GMFR leveled off for the 100-μg C. difficile group (Day 30: 3.76; Day 37: 3.89; Month 2: 3.61). This booster response was maximal at Day 37 (7 days after Dose 3) for the 200-μg C. difficile group. The GMFRs decreased to 2.40 for the 100-μg C. difficile group and to 4.44 for the 200-μg C. difficile group at Month 13.

Between Day 8 and Month 13, toxin A- and toxin B-specific neutralizing antibody GMFRs were higher for the 200-μg C. difficile group compared with the 100-μg C. difficile group.

Toxin A- and toxin B-specific neutralizing antibody GMFRs by subjects' baseline serostatus for each time point was assessed for the day regimen. Among the baseline seronegative subjects, an increase in toxin A-specific neutralizing antibody GMFRs was observed at Day 30 (100-μg C. difficile group, 2.01; 200-μg C. difficile group, 2.95). After Dose 3, a booster response was evident at Day 37 (100-μg C. difficile group, 4.48; 200-μg C. difficile group, 6.95). This booster response was maximal at Day 37. The GMFRs decreased to 1.28 for the 100-μg C. difficile group and to 1.75 for the 200-μg C. difficile group at Month 13. After Dose 2, an increase in toxin B-specific neutralizing antibody GMFRs was observed at Day 15 (100-μg C. difficile group, 2.57; 200-μg C. difficile group, 3.81). After Dose 3, GMFRs leveled off for the 100-μg C. difficile group (Day 30: 2.37; Day 37: 2.56; Month 2: 2.42) and the 200-μg C. difficile group (Day 30: 3.68; Day 37: 4.55; Month 2: 4.22). The GMFRs decreased to 2.01 for the 100-μg C. difficile group and to 4.00 for the 200-μg C. difficile group at Month 13.

Among the baseline seropositive subjects, an increase in toxin A-specific neutralizing antibody GMFRs was observed at Day 30 (100-μg C. difficile group, 2.47; 200-μg C. difficile group, 1.20). After Dose 3, a booster response was evident at Day 37 (100-μg C. difficile group, 3.97; 200-μg C. difficile group, 3.68). This booster response was maximal at Day 37. The GMFRs decreased to 0.99 for the 100-μg C. difficile group and to 0.81 for the 200-μg C. difficile group at Month 13. After Dose 2, a marked increase in toxin B-specific neutralizing antibody GMFRs was observed at Day 15 (100-μg C. difficile group, 32.18; 200-μg C. difficile group, 32.14). A booster response was not evident after Dose 3 as the GMFRs leveled off for the 100-μg C. difficile group (Day 30: 22.86; Day 37: 19.74; Month 2: 17.23) and the 200-μg C. difficile group (Day 30: 27.50; Day 37: 28.23; Month 2: 25.54). The GMFRs decreased to 4.72 for the 100-μg C. difficile group and to 6.70 for the 200-μg C. difficile group at Month 13.

Between Day 8 and Month 13, toxin A-specific neutralizing antibody GMFRs for the baseline seronegative subjects were higher for the 200-μg C. difficile group compared with the 100-μg C. difficile group. Between Day 8 and Month 13, toxin A-specific neutralizing antibody GMFRs for the baseline seropositive subjects were lower for the 200-μg C. difficile group compared with the 100-μg C. difficile group, except at Month 4. Toxin A-specific neutralizing antibody GMFRs were higher for the baseline seronegative subjects in both dose groups compared with the baseline seropositive subjects. Between Day 8 and Month 13, toxin B-specific neutralizing antibody GMFRs for baseline seronegative and seropositive subjects were higher for the 200-μg C. difficile group compared with the 100-μg C. difficile group, except at Day 15 for the baseline seropositive subjects. Between Day 8 and Month 13, toxin B-specific neutralizing antibody GMFRs were higher for the baseline seropositive subjects compared with the baseline seronegative subjects.

The proportions of subjects achieving defined fold rises from baseline in toxin A-, toxin B-, and both toxin A- and toxin B-specific neutralizing antibody levels at Day 37 and at the other blood sampling time points were summarized.

Overall, greater proportions of subjects achieving defined fold rises from baseline toxin A-specific neutralizing antibody levels were observed for the 200-μg C. difficile group compared with the 100-μg C. difficile group. For the 100-μg C. difficile group, increases in proportions of subjects reaching a ≥4-fold rise were observed from Day 15, with 43 (25.1%) subjects reaching a ≥8-fold rise at Day 37. For the 100-μg C. difficile group, 15 (8.8%) subjects reached a ≥16-fold rise and 6 (3.5%) subjects reached a ≥32-fold rise in toxin A-specific neutralizing antibody levels at Day 37. For the 200-μg C. difficile group, increases in proportions of subjects reaching a ≥4-fold rise were observed from Day 15, with a marked increase in the proportion of subjects (67 [40.0%]) reaching a ≥8-fold rise at Day 37. For the 200-μg C. difficile group, 23 (14.1%) subjects reached a ≥16-fold rise and 15 (9.2%) subjects reached a ≥32-fold rise in toxin A-specific neutralizing antibody levels at Day 15.

Overall, similar proportions of subjects achieving defined fold rises from baseline toxin B-specific neutralizing antibody levels were observed for the 100-μg and 200-μg C. difficile groups up to Month 4. At Months 7 and 13, greater proportions of subjects achieving defined fold rises from baseline toxin A-specific neutralizing antibody levels were observed for the 200-μg C. difficile group compared with the 100-μg C. difficile group. For the 100-μg C. difficile group, increases in proportions of subjects reaching a ≥4-fold rise were observed from Day 15, with 57 (33.3%) subjects reaching a ≥8-fold rise at Day 15 and Day 30. For the 100-μg C. difficile group, 45 (26.3%) subjects reached a ≥16-fold rise and 35 (20.5%) subjects reached a ≥32-fold rise in toxin A-specific neutralizing antibody levels at Day 15. For the 200-μg C. difficile group, increases in proportions of subjects reaching a ≥4-fold rise were observed from Day 15, with a marked increase in the proportion of subjects (66 [40.2%]) reaching a ≥8-fold rise at Day 37. For the 200-μg C. difficile group, 61 (37.4%) subjects reached a ≥16-fold rise and 45 (27.4%) subjects reached a ≥32-fold rise in toxin A-specific neutralizing antibody levels at Day 30 and Day 37.

Overall, results for the proportions of subjects achieving defined fold rises from baseline for both toxin A- and toxin B-specific neutralizing antibody levels were similar to those of toxin A.

Reverse Cumulative Distribution Curves (RCDCs) displaying data for toxin A-specific neutralizing antibody levels measured at Day 1, Day 8, Day 15, Day 30, Day 37, Month 2, Month 4, Month 7, and Month 13 by vaccine group were assessed for the day regimen. RCDCs displaying data for toxin B-specific neutralizing antibody levels measured at Day 1, Day 8, Day 15, Day 30, Day 37, Month 2, Month 4, Month 7, and Month 13 by vaccine group were assessed for the day regimen. Overall, the observed curve shifted to the right for the vaccinated groups, indicating that immunization with the toxoid vaccine increased titer levels over that seen with placebo. This is consistent with the patterns shown in GMCs and the proportions of subjects achieving the specified thresholds.

### Example 7

### Phase 2 Study, 3-Dose Regimen at Days 1, 8, and 30 (Day Regimen) or Months 0, 1, and 6 (Month Regimen) (B5091009)-Immunogenicity Evaluation, Results by Age Group

The immune response by age group (65 to 69 years, 70 to 74 years, 75 to 79 years, and 80 to 85 years) was also evaluated.

In the month regimen, at Month 7, the proportions of subjects in the 100-μg C. difficile group achieving both toxin A- and toxin B-specific neutralizing antibody titers≥the specified thresholds were 80.0% of 70 subjects aged 65 to 69 years, 68.8% of 48 subjects aged 70 to 74 years, 73.5% of 34 subjects aged 75 to 79 years, and 63.6% of 11 subjects aged 80 to 85 years. The corresponding proportions of subjects in the 200-μg C. difficile group were 90.6% of 64 subjects aged 65 to 69 years, 83.9% of 56 subjects aged 70 to 74 years, 89.7% of 29 subjects aged 75 to 79 years, and 55.6% of 9 subjects aged 80 to 85 years.

In the month regimen, at Month 7, toxin A-specific neutralizing antibody GMCs for the subjects in the 100-μg C. difficile group were 1304 neutralization units/mL for the subjects aged 65 to 69 years, 1182 neutralization units/mL for the subjects aged 70 to 74 years, 1254 neutralization units/mL for the subjects aged 75 to 79 years, and 1132 neutralization units/mL for the subjects aged 80 to 85 years. The corresponding GMCs for the subjects in the 200-μg C. difficile group were 1690 neutralization units/mL for the subjects aged 65 to 69 years, 1073 neutralization units/mL for the subjects aged 70 to 74 years, 1627 neutralization units/mL for the subjects aged 75 to 79 years, and 925 neutralization units/mL for the subjects aged 80 to 85 years.

In the month regimen, at Month 7, toxin B-specific neutralizing antibody GMCs for the subjects in the 100-μg C. difficile group were 7608 neutralization units/mL for the subjects aged 65 to 69 years, 5688 neutralization units/mL for the subjects aged 70 to 74 years, 5446 neutralization units/mL for the subjects aged 75 to 79 years, and 4178 neutralization units/mL for the subjects aged 80 to 85 years. The corresponding GMCs for the subjects in the 200-μg C. difficile group were 11835 neutralization units/mL for the subjects aged 65 to 69 years, 8750 neutralization units/mL for the subjects aged 70 to 74 years, 10533 neutralization units/mL for the subjects aged 75 to 79 years, and 2608 neutralization units/mL for the subjects aged 80 to 85 years.

In the month regimen, at Month 7, the proportions of baseline seronegative subjects in the 100-μg C. difficile group achieving both toxin A- and toxin B-specific neutralizing antibody titers≥the specified thresholds were 73.9% of 46 subjects aged 65 to 69 years, 64.1% of 39 subjects aged 70 to 74 years, 67.9% of 28 subjects aged 75 to 79 years, and 62.5% of 8 subjects aged 80 to 85 years. The corresponding proportions of subjects in the 200-μg C. difficile group were 91.3% of 46 subjects aged 65 to 69 years, 81.1% of 37 subjects aged 70 to 74 years, 86.4% of 22 subjects aged 75 to 79 years, and 55.6% of 9 subjects aged 80 to 85 years.

In the month regimen, at Month 7, the proportions of baseline seropositive subjects in the 100-μg C. difficile group achieving both toxin A- and toxin B-specific neutralizing antibody titers≥the specified thresholds were 100.0% of 2 subjects aged 65 to 69 years, 100.0% of 1 subject aged 70 to 74 years, not estimable for subjects aged 75 to 79 years, and not estimable for subjects aged 80 to 85 years. The corresponding proportions of subjects in the 200-μg C. difficile group were 66.7% of 3 subjects aged 65 to 69 years, 100.0% of 2 subjects aged 70 to 74 years, not estimable for subjects aged 75 to 79 years, and not estimable for subjects aged 80 to 85 years.

In the month regimen, at Month 7, the proportions of baseline seronegative subjects in the 100-μg C. difficile group achieving both toxin A- and toxin B-specific neutralizing antibody titers≥the specified thresholds were 73.9% of 46 subjects aged 65 to 69 years, 64.1% of 39 subjects aged 70 to 74 years, 67.9% of 28 subjects aged 75 to 79 years, and 62.5% of 8 subjects aged 80 to 85 years. The corresponding proportions of subjects in the 200-μg C. difficile group were 91.3% of 46 subjects aged 65 to 69 years, 81.1% of 37 subjects aged 70 to 74 years, 86.4% of 22 subjects aged 75 to 79 years, and 55.6% of 9 subjects aged 80 to 85 years.

In the month regimen, at Month 7, the proportions of baseline seropositive subjects in the 100-μg C. difficile group achieving both toxin A- and toxin B-specific neutralizing antibody titers≥the specified thresholds were 100.0% of 2 subjects aged 65 to 69 years, 100.0% of 1 subject aged 70 to 74 years, not estimable for subjects aged 75 to 79 years, and not estimable for subjects aged 80 to 85 years. The corresponding proportions of subjects in the 200-μg C. difficile group were 66.7% of 3 subjects aged 65 to 69 years, 100.0% of 2 subjects aged 70 to 74 years, not estimable for subjects aged 75 to 79 years, and not estimable for subjects aged 80 to 85 years.

In the month regimen, at Month 7, the proportions of baseline seronegative subjects in the 100-μg C. difficile group achieving ≥4-fold rise from baseline in both toxin A- and toxin B-specific neutralizing antibody titers≥the specified thresholds were 84.8% of 46 subjects aged 65 to 69 years, 87.2% of 39 subjects aged 70 to 74 years, 78.6% of 28 subjects aged 75 to 79 years, and 100.0% of 8 subjects aged 80 to 85 years. The corresponding proportions of subjects in the 200-μg C. difficile group were 91.3% of 46 subjects aged 65 to 69 years, 81.1% of 37 subjects aged 70 to 74 years, 100.0% of 22 subjects aged 75 to 79 years, and 77.8% of 9 subjects aged 80 to 85 years.

In the month regimen, at Month 7, the proportions of baseline seropositive subjects in the 100-μg C. difficile group achieving ≥4-fold rise from baseline in both toxin A- and toxin B-specific neutralizing antibody titers≥the specified thresholds were 100.0% of 2 subjects aged 65 to 69 years, 100% of 1 subject aged 70 to 74 years, not estimable for subjects aged 75 to 79 years, and not estimable for subjects aged 80 to 85 years. The corresponding proportions of subjects in the 200-μg C. difficile group were 66.7% of 3 subjects aged 65 to 69 years, 50.0% of 2 subjects aged 70 to 74 years, not estimable for subjects aged 75 to 79 years, and not estimable for subjects aged 80 to 85 years.

In the day regimen, at Day 37, the proportions of subjects in the 100-μg C. difficile group achieving both toxin A- and toxin B-specific neutralizing antibody titers≥the specified thresholds were 23.0% of 74 subjects aged 65 to 69 years, 30.2% of 53 subjects aged 70 to 74 years, 28.6% of 28 subjects aged 75 to 79 years, and 25.0% of 16 subjects aged 80 to 85 years. The corresponding proportions of subjects in the 200-μg C. difficile group were 38.0% of 71 subjects aged 65 to 69 years, 30.0% of 50 subjects aged 70 to 74 years, 48.3% of 29 subjects aged 75 to 79 years, and 53.3% of 15 subjects aged 80 to 85 years.

In the day regimen, at Day 37, toxin A-specific neutralizing antibody GMCs for the subjects in the 100-μg C. difficile group were 422 neutralization units/mL for the subjects aged 65 to 69 years, 389 neutralization units/mL for the subjects aged 70 to 74 years, 282 neutralization units/mL for the subjects aged 75 to 79 years, and 262 neutralization units/mL for the subjects aged 80 to 85 years. The corresponding GMCs for the subjects in the 200-μg C. difficile group were 569 neutralization units/mL for the subjects aged 65 to 69 years, 558 neutralization units/mL for the subjects aged 70 to 74 years, 496 neutralization units/mL for the subjects aged 75 to 79 years, and 616 neutralization units/mL for the subjects aged 80 to 85 years.

In the day regimen, at Day 37, toxin B-specific neutralizing antibody GMCs for the subjects in the 100-μg C. difficile group were 653 neutralization units/mL for the subjects aged 65 to 69 years, 809 neutralization units/mL for the subjects aged 70 to 74 years, 566 neutralization units/mL for the subjects aged 75 to 79 years, and 1309 neutralization units/mL for the subjects aged 80 to 85 years. The corresponding GMCs for the subjects in the 200-μg C. difficile group were 1192 neutralization units/mL for the subjects aged 65 to 69 years, 805 neutralization units/mL for the subjects aged 70 to 74 years, 2404 neutralization units/mL for the subjects aged 75 to 79 years, and 1449 neutralization units/mL for the subjects aged 80 to 85 years.

In the day regimen, at Day 37, the proportions of baseline seronegative subjects in the 100-μg C. difficile group achieving both toxin A- and toxin B-specific neutralizing antibody titers≥the specified thresholds were 11.9% of 59 subjects aged 65 to 69 years, 13.2% of 38 subjects aged 70 to 74 years, 9.1% of 22 subjects aged 75 to 79 years, and 9.1% of 11 subjects aged 80 to 85 years. The corresponding proportions of subjects in the 200-μg C. difficile group were 22.4% of 49 subjects aged 65 to 69 years, 21.1% of 38 subjects aged 70 to 74 years, 36.4% of 22 subjects aged 75 to 79 years, and 46.2% of 13 subjects aged 80 to 85 years.

In the day regimen, at Day 37, the proportions of baseline seropositive subjects in the 100-μg C. difficile group achieving both toxin A- and toxin B-specific neutralizing antibody titers≥the specified thresholds were 100.0% of 2 subjects aged 65 to 69 years, 50.0% of 2 subjects aged 70 to 74 years, not estimable for subjects aged 75 to 79 years, and not estimable for subjects aged 80 to 85 years. The corresponding proportions of subjects in the 200-μg C. difficile group were 100.0% of 3 subjects aged 65 to 69 years, 100.0% of 3 subjects aged 70 to 74 years, not estimable for subjects aged 75 to 79 years, and not estimable for subjects aged 80 to 85 years.

In the day regimen, at Day 37, the proportions of baseline seronegative subjects in the 100-μg C. difficile group achieving both toxin A- and toxin B-specific neutralizing antibody titers≥the specified thresholds were 11.9% of 59 subjects aged 65 to 69 years, 13.2% of 38 subjects aged 70 to 74 years, 9.1% of 22 subjects aged 75 to 79 years, and 9.1% of 11 subjects aged 80 to 85 years. The corresponding proportions of subjects in the 200-μg C. difficile group were 22.4% of 49 subjects aged 65 to 69 years, 21.1% of 38 subjects aged 70 to 74 years, 36.4% of 22 subjects aged 75 to 79 years, and 46.2% of 13 subjects aged 80 to 85 years.

In the day regimen, at Day 37, the proportions of baseline seropositive subjects in the 100-μg C. difficile group achieving both toxin A- and toxin B-specific neutralizing antibody titers≥the specified thresholds were 100.0% of 2 subjects aged 65 to 69 years, 50.0% of 2 subjects aged 70 to 74 years, not estimable for subjects aged 75 to 79 years, and not estimable for subjects aged 80 to 85 years. The corresponding proportions of subjects in the 200-μg C. difficile group were 100.0% of 3 subjects aged 65 to 69 years, 100.0% of 3 subjects aged 70 to 74 years, not estimable for subjects aged 75 to 79 years, and not estimable for subjects aged 80 to 85 years.

In the day regimen, at Day 37, the proportions of baseline seronegative subjects in the 100-μg C. difficile group achieving ≥4-fold rise from baseline in both toxin A- and toxin B-specific neutralizing antibody titers≥the specified thresholds were 15.3% of 59 subjects aged 65 to 69 years, 18.4% of 38 subjects aged 70 to 74 years, 18.2% of 22 subjects aged 75 to 79 years, and 18.2% of 11 subjects aged 80 to 85 years. The corresponding proportions of subjects in the 200-μg C. difficile group were 24.5% of 49 subjects aged 65 to 69 years, 26.3% of 38 subjects aged 70 to 74 years, 36.4% of 22 subjects aged 75 to 79 years, and 46.2% of 13 subjects aged 80 to 85 years.

In the day regimen, at Day 37, the proportions of baseline seropositive subjects in the 100-μg C. difficile group achieving ≥4-fold rise from baseline in both toxin A- and toxin B-specific neutralizing antibody titers≥the specified thresholds were 100.0% of 2 subjects aged 65 to 69 years, 50.0% of 2 subjects aged 70 to 74 years, not estimable for subjects aged 75 to 79 years, and not estimable for subjects aged 80 to 85 years. The corresponding proportions of subjects in the 200-μg C. difficile group were 33.3% of 3 subjects aged 65 to 69 years, 0.0% of 3 subjects aged 70 to 74 years, not estimable for subjects aged 75 to 79 years, and not estimable for subjects aged 80 to 85 years.

### Example 8

### A Phase 2, Placebo-Controlled, Randomized, Observer-Blinded Study to Evaluate the Safety, Tolerability, and Immunogenicity of Two 3 Dose Regimens of a Clostridium difficile Vaccine in Healthy Adults Aged 65 to 85 Years, Through 12 Months Post Dose 3

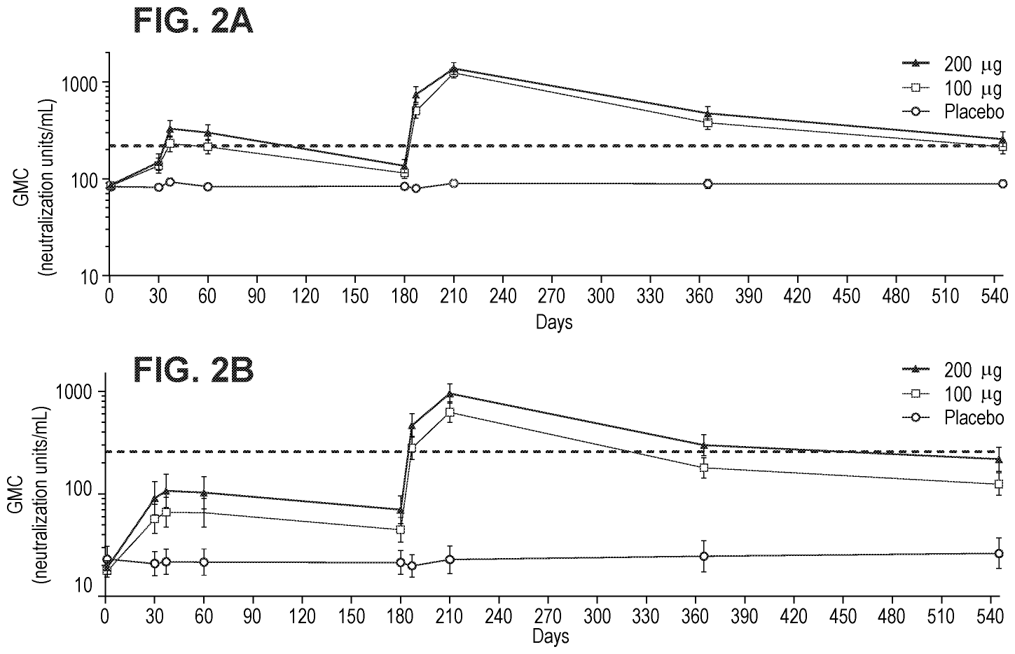

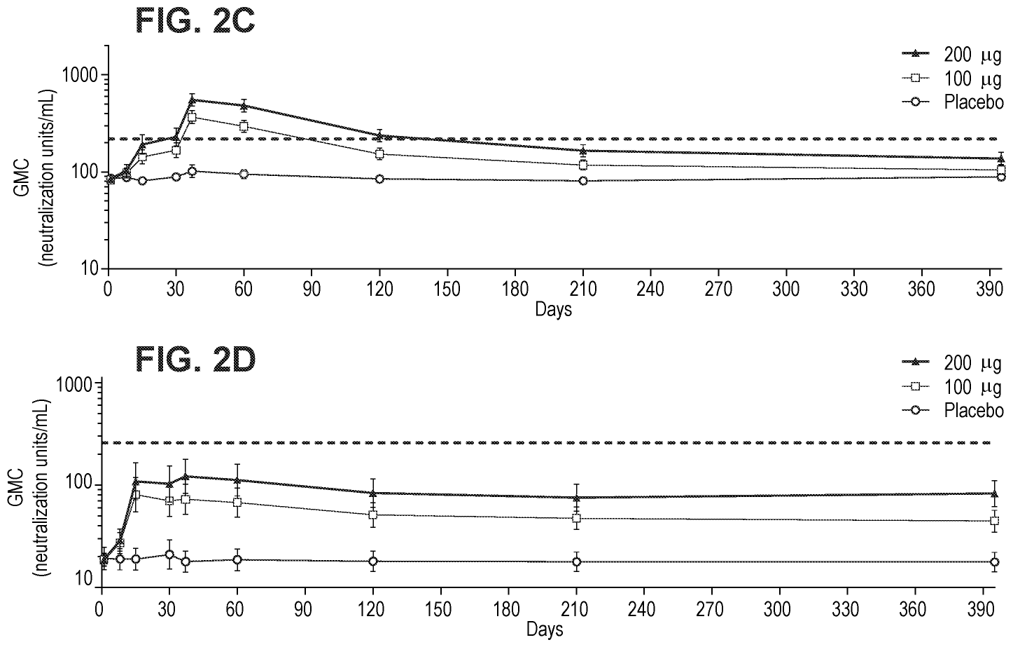

Clostridium difficile (C. difficile) is a common cause of antibiotic-associated nosocomial diarrhea. To date, there is no vaccine available to prevent C. difficile infection (CDI). In this phase 2 study we explore the safety, tolerability and immunogenicity of a toxoid based C. difficile vaccine in 855 healthy immunocompetent adults aged 65 to 85 years in the United States Methods. The originally planned stage of this trial was conducted from 16 Jul. 2015 to 7 Mar. 2017. Subjects were enrolled and randomized to receive one of two antigen dose levels (100 μg or 200 μg) or placebo and one of two 3-dose regimens: Days 1, 8 & 30 (Day regimen) or Month 0, 1 & 6 (Month regimen). Immunogenicity testing was conducted on blood samples obtained at each of nine study visits. Local and systemic reactogenicity was collected after each vaccination. Non-serious Adverse Event (AE) reporting occurred through 1 month post dose 3. Serious Adverse Event (SAE) reporting occurred through 6 months post dose 3. 855 healthy, immunocompetent subjects were enrolled into the study and randomly assigned in parallel in a 3:3:1 ratio to receive 3 doses of C. difficile vaccine (100 μg or 200 μg total toxoid) or placebo (saline) in 1 of 2 dosing regiments: Days 1, 8 and 30 (Day regimen) or Months 0, 1 and 6 (Month regimen). Immunogenicity testing was conducted on blood samples obtained at each of 9 study visits: days 1, 8, 15, 30, 37, months 2, 4, 7, and 13 for the Day regimen & days 1, 30, 37, 187, months 2, 6, 7, 12 and 18 for the Month regimen. Toxin A and toxin B specific neutralizing immunoglobulin G (IgG) concentrations (neutralization units/mL) were measured at each scheduled study visit. Geometric Mean Concentrations (GMC) were calculated and neutralization thresholds established for toxin A and toxin B—(FIG. 2A, FIG. 2B, FIG. 2C, and FIG. 2D) Local reactions and systemic events were collected by e-diary for 14 days after each vaccination (7 days post dose 1 for the Day regimen). Adverse Event reporting occurred from signing of informed consent through 1 month post dose 3. Serious Adverse Event (SAE) reporting occurred through 6 months post dose 3. Safety, reactogenicity and immunogenicity were descriptively analyzed within each vaccination regimen by dose (100 μg and 200 μg) and placebo.

Results. Overall immunogenicity was greater in the 200 μg Month regimen after 3 doses. Local reactogenicity demonstrated an increase post dose #2 in the Day regimen. Adverse event rates in the Month regimen were numerically higher due to an additional 5 month follow up compared to the Day regimen.

Peak antibody immune response to vaccination was observed at month 7 and day 37 following dose 3 for the Month and Day regimen respectively—(FIG. 2A, FIG. 2B, FIG. 2C, and FIG. 2D).

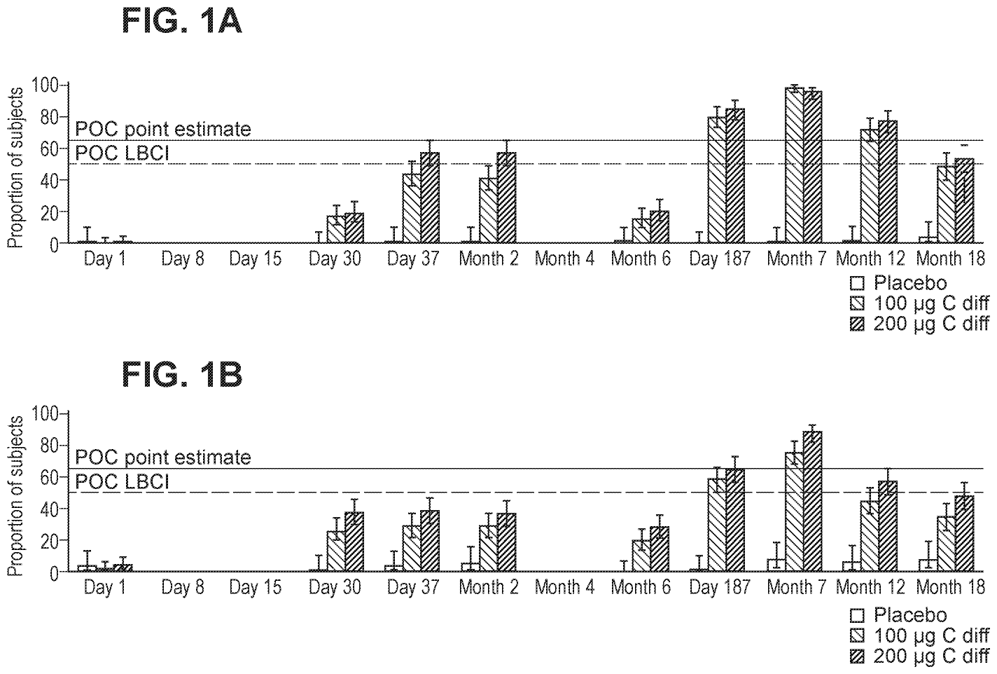

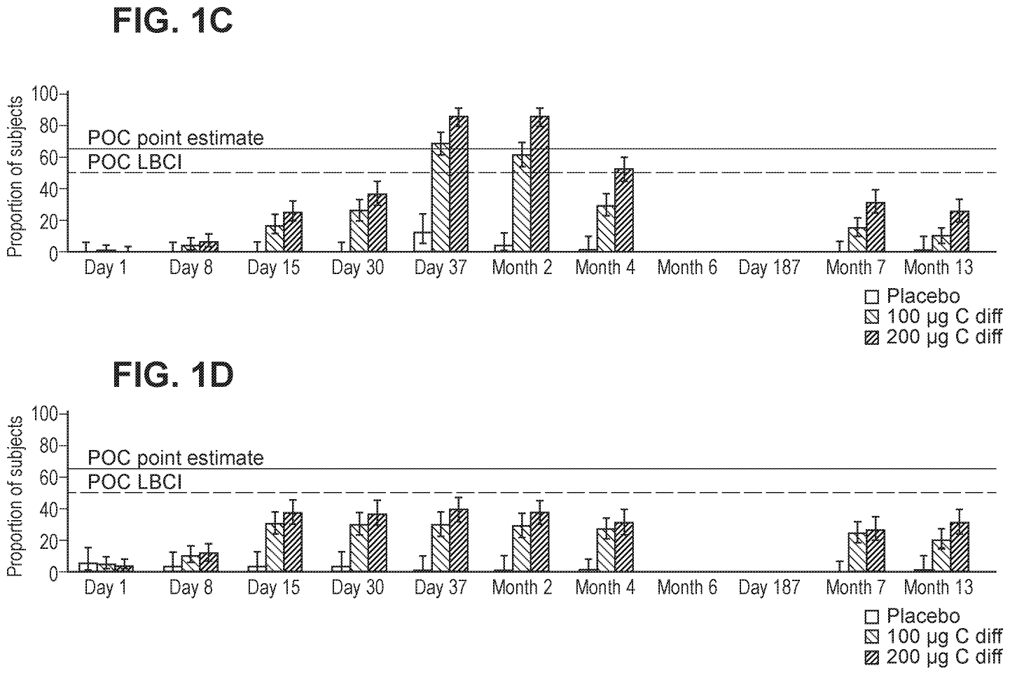

12 months post dose 3, the Month regimen demonstrates a higher proportion of subjects above predefined neutralization threshold compared to the Day regimen irrespective of dose group—(FIG. 1A, FIG. 1B, FIG. 1C, and FIG. 1D).

At 12 months post dose 3, Geometric Mean Concentrations (GMCs) for the Month regimen are higher compared to Day regimen GMCs for toxin B (4 fold higher) and toxin A (2 fold higher)—(FIG. 2A, FIG. 2B, FIG. 2C, and FIG. 2D).

At 12 months post dose 3, Toxin A GMCs remain above neutralization threshold in the 200 μg Month regimen—(FIG. 2A and FIG. 2B).

Toxin B GMCs are above neutralization threshold in the 200 μg Month regimen until approximately 9 months post dose 3—(FIG. 2A and FIG. 2B).

Pain is the most common local reaction reported; the incidence of any redness and swelling was <10% after each dose in both regimens

No Grade 4 local or systemic reactogenicity were reported during the study

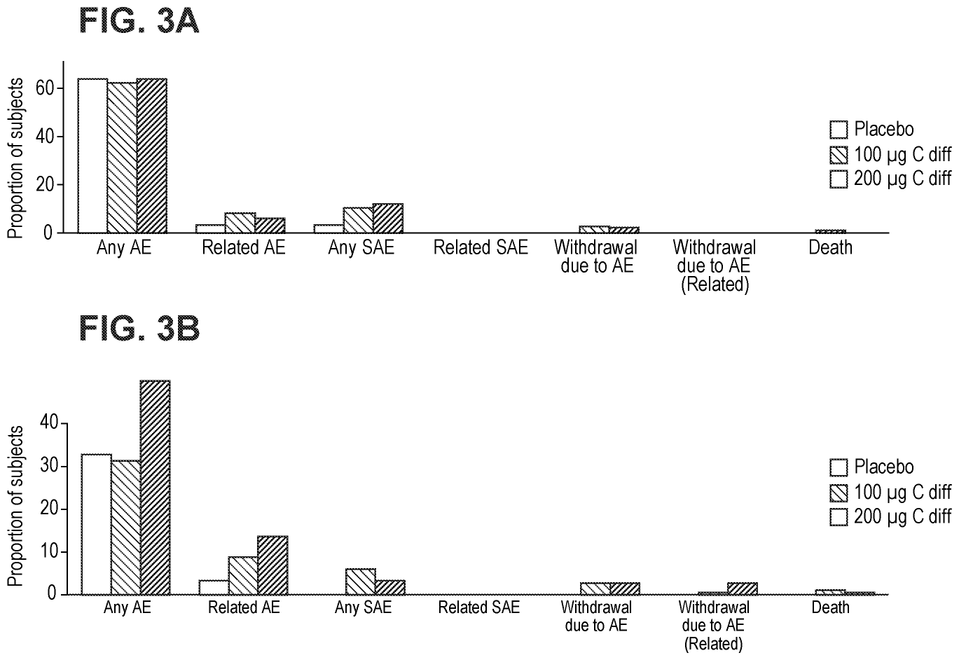

In the Month regimen, no significant numerical difference was observed among aggregated adverse events between dose groups and placebo—(FIG. 3A and FIG. 3B).

There were no Serious Adverse Events (SAE) related to C. difficile vaccination reported during the trial among any regimen or dose group—(FIG. 3A and FIG. 3B).

Conclusions. The Month regimen is more immunogenic than the Day regimen after 3 doses of C. difficile vaccination. Increased local reactogenicity was observed after dose 2 when given at day 8, particularly at the 200 μg dose level. Related adverse events, serious adverse events and withdrawals due to adverse events were numerically greater in the active vaccine groups but there were no discernible patterns to suggest a safety concern. Overall adverse event frequency and type were characteristic of this age cohort in the general population. The C. difficile vaccination was immunogenic and well tolerated. The Clostridium difficile vaccine candidate is highly immunogenic, well tolerated and demonstrates an acceptable safety profile. The 200 μg Month regimen demonstrated greater immunogenicity overall and was selected for phase 3 development.

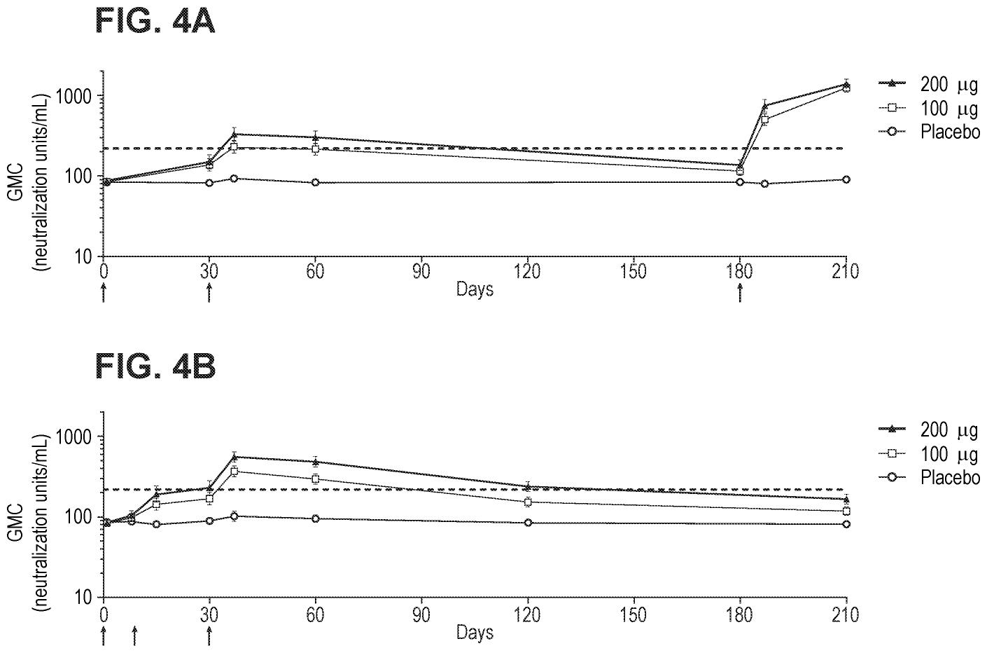

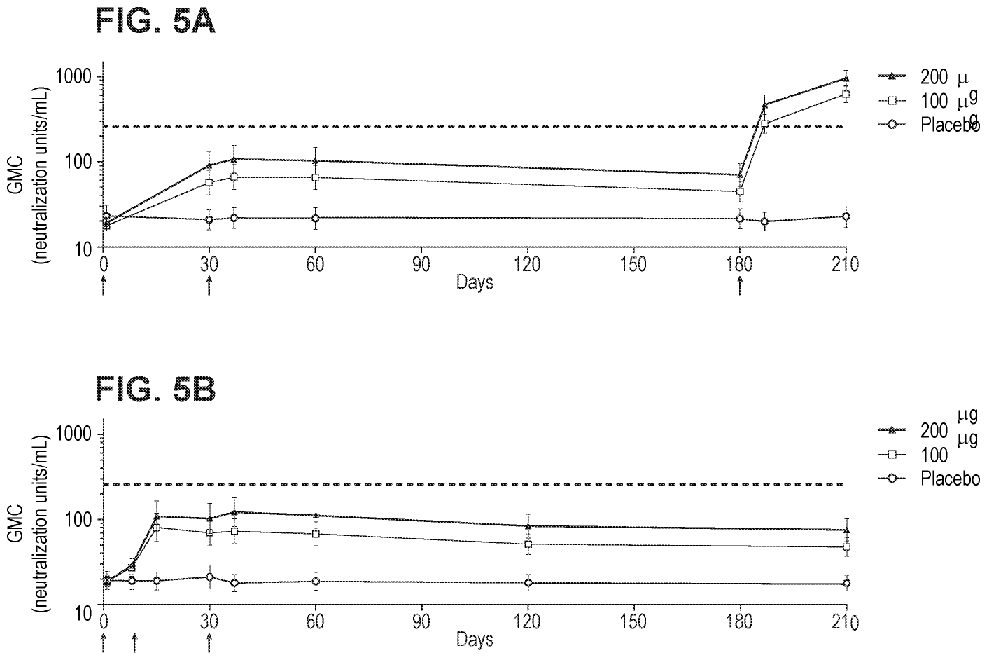

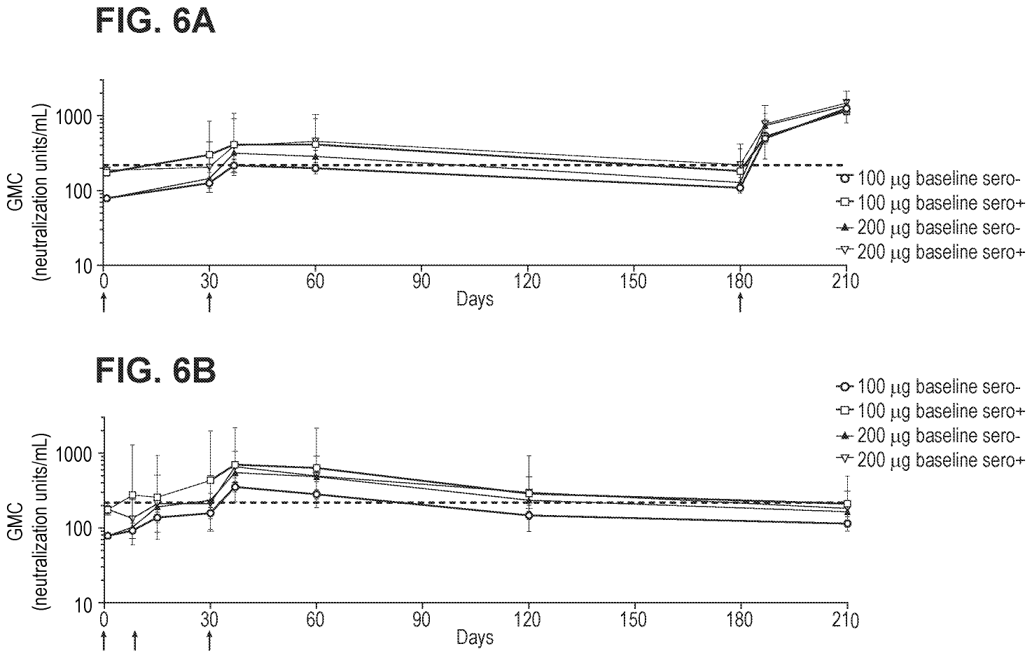

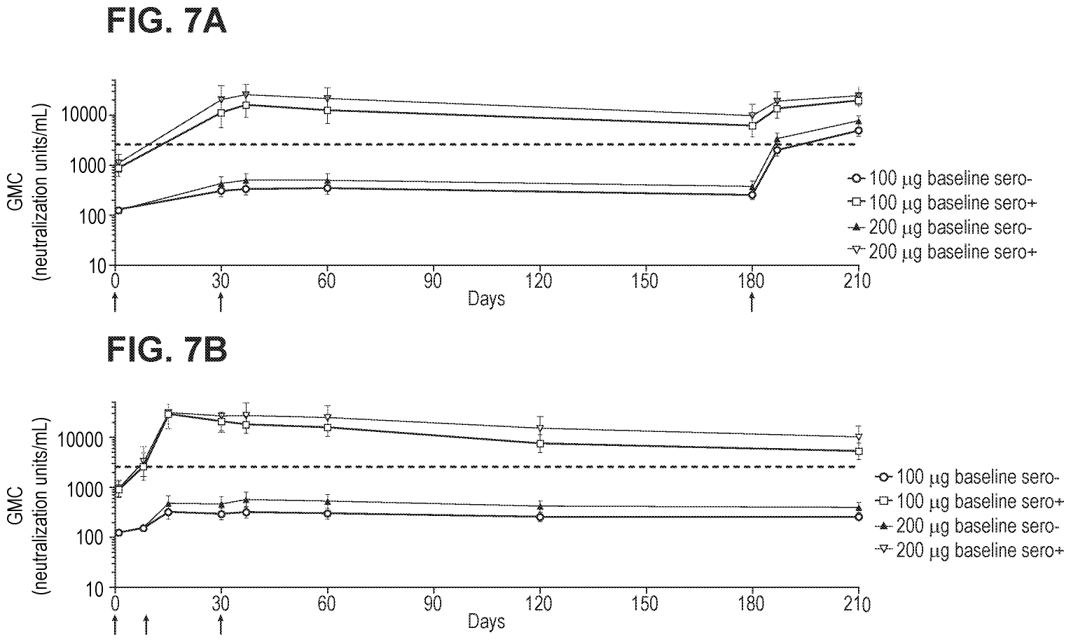

The 200 μg dose level was consistently more immunogenic than 100 μg dose level. The Day regimen drives a superior earlier immune response at Day 37/Month 2 for toxin A but not for toxin B. See, for example, FIG. 4A, FIG. 4B, FIG. 5A, and FIG. 5B. For toxin A, baseline seropositivity contributes to the magnitude of the early immune response (particularly for the 100 μg dose level) but, in both regimens, there is little difference post-dose 3. For toxin B baseline seronegative subjects (˜80% of total), it is only the 3rd dose given at Month 6 that results in significant proportion achieving threshold. See, for example, FIG. 6A, FIG. 6B, FIG. 7A, and FIG. 7B. For all systemic events, no significant differences were observed between placebo and the C. difficile vaccine, or between the 100 μg and 200 μg dose levels. Related adverse events, serious adverse events and withdrawals due to adverse events were numerically greater in the active vaccine groups but there were no discernible patterns to suggest a safety concern. The 200 μg dose level administered at 0, 1 and 6 months was selected for Phase 3.

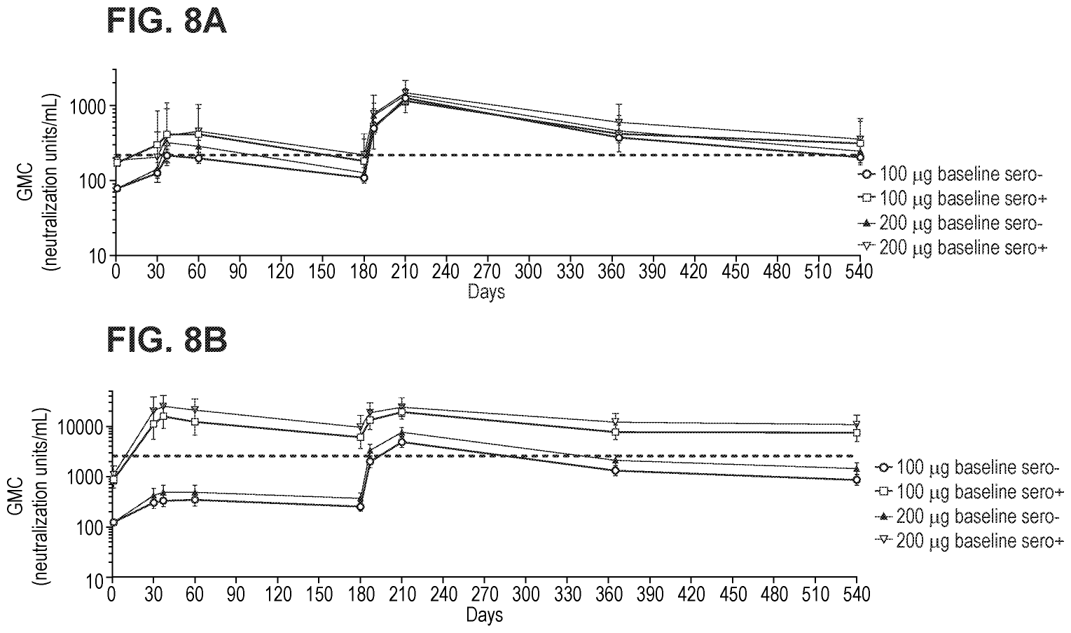

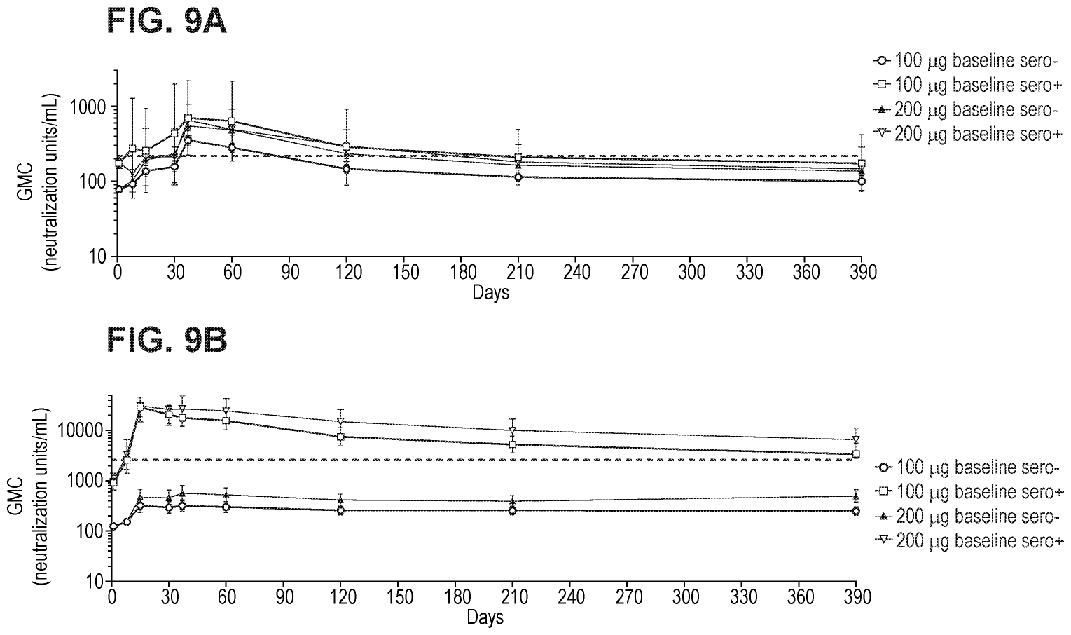

With respect to the results at month 12 post-dose 3, at 12 months post dose #3, approximately 50% of subjects are above pre-defined thresholds in both dose groups for neutralizing against Toxin A. The Month regimen, 200 μg dose group geometric mean concentrations (GMCs) are above the neutralization threshold against Toxin A at 12 months following Dose #3. At 12 months post dose #3, 47% of subjects are above pre-defined thresholds in the 200 μg dose group of the Month regimen for neutralizing against Toxin B. GMC(s) of seropositive are above neutralization threshold in both regimens for neutralizing against Toxin B. With respect to the Month regimen, toxin B GMCs are above the neutralization threshold 6 months post dose #3 (2993 neutralization units/mL) and remain similar (2178 neutralization units/mL) 12 months post dose #3. See, for example FIGS. 1A-D, FIGS. 2A-D, FIG. 8A, FIG. 8B, and FIG. 9A, and FIG. 9B.

### Example 9

### A Phase 1, Placebo-Controlled, Randomized, Observer-Blinded Study to Evaluate the Safety, Tolerability, and Immunogenicity of Two 3-Dose Regimens of Clostridium difficile Vaccine Administered in Healthy Japanese Adults Aged 65 to 85 Years (B5091010)

This was a Phase 1, placebo-controlled, randomized, observer-blinded study to assess the safety, tolerability, and immunogenicity of 2 antigen dose levels of the aluminum hydroxide-containing vaccine (i.e., 100-μg and 200-μg) in 2 different dosing regimens (Months 0, 1, and 6 [month regimen] or Days 1, 8, and 30 [day regimen]) in healthy Japanese adults aged 65 to 85 years.

A total of 128 healthy Japanese adults, aged 65 to 85 years, were to be enrolled. Subjects were randomly assigned in a 3:3:2 ratio to receive C. difficile vaccine (100 μg or 200 μg total toxoid) or placebo (saline) in each dosing regimen.

Subjects were followed for 6 months after receipt of their last vaccination. Therefore, subjects assigned to the month regimen participated for approximately 12 months and those assigned to and completing the day regimen participated for approximately 7 months. This study was planned to be completed in approximately 14 months. The end of the study was the last visit of the last subject.

The C. difficile vaccine had not been previously evaluated in Japanese subjects. Therefore the present first-in-Japanese Study B5091010 was designed similar to Study B5091009 to evaluate the safety, tolerability, and immunogenicity of 2 antigen dose levels (100 μg and 200 μg total toxoid) of aluminum hydroxide-containing C. difficile vaccine when administered as two 3-dose regimens (either Days 1, 8, and 30 or Months 0, 1, and 6) to healthy Japanese adults aged 65 to 85 years.

During the period when Study B5091010 was underway, the Phase 2 Study B5091009 was analyzed and demonstrated that both regimens and both dose levels administered were generally well tolerated. Local reactions were predominantly mild to moderate, with injection site pain being the most frequent manifestation. After Dose 2, local reactogenicity was greater when the vaccine was administered at Day 8 compared to Month 1, particularly for the 200-μg dose level. Systemic events were also predominantly mild to moderate and the incidences of individual events were similar among the placebo group, the 100-μg dose group, and the 200-μg dose group. Within each regimen, the overall adverse event (AE) incidence rates were also similar among the placebo, 100-μg, and 200-μg dose groups. For both regimens, serious adverse events (SAEs) were numerically higher in the 100-μg and 200-μg dose groups than in the placebo group. However, there was no pattern to these events and no safety concern was identified. Both studied dose levels resulted in substantial neutralizing antitoxin A and B titers, with the immunogenicity profile following 3 doses administered at Months 0, 1, and 6 being preferred. In addition, the 200-μg dose level was more immunogenic than the 100-μg dose level. On this basis, it was decided to progress into Phase 3 development with the 200-μg dose level administered at Months 0, 1, and 6.

For each dosing regimen, subjects were randomized to 1 of 3 study groups as listed in Table 4. Approximately 24 subjects were planned to receive the C. difficile vaccine at either 100 or 200 μg total toxoid dose level and approximately 16 subjects to receive placebo (saline).

TABLE 4
Vaccine Groups and Planned Number of Subjects
per Group and per Dose Regimen

| Vaccine | | Dosing | Number of |
|---|---|---|---|
| Group | Vaccine Formulation Description | Regimen | Subjects |
| 1 | Aluminum hydroxide-containing | Months 0, 1, | 24 |
| | Clostridium difficile vaccine | 6 | |
| | (100-μg antigen dose) | | |
| 2 | Aluminum hydroxide-containing | Months 0, 1, | 24 |
| | C difficile vaccine (200-μg | 6 | |
| | antigen dose) | | |
| 3 | Placebo (saline) | Months 0, 1, | 16 |
| | | 6 | |
| 4 | Aluminum hydroxide-containing | Days 1, 8, | 24 |
| | C difficile vaccine (100-μg | 30 | |
| | antigen dose) | | |
| 5 | Aluminum hydroxide-containing | Days 1, 8, | 24 |
| | C difficile vaccine (200-μg | 30 | |
| | antigen dose) | | |
| 6 | Placebo (saline) | Days 1, 8, | 16 |
| | | 30 | |
| Total | | | 128 |

Serum samples were obtained for immunogenicity testing. For the month regimen, on Day 1 (prior to administration of vaccine), Day 15 (14 days after Dose 1), Day 30 (immediately before Dose 2), Day 37 (7 days after Dose 2), Month 2 (1 month after Dose 2), Month 6 (immediately before Dose 3), Day 187 (7 days after Dose 3), Month 7 (1 month after Dose 3), and Month 12 (6 months after Dose 3).

For the day regimen, on Day 1 (prior to administration of vaccine), Day 8 (immediately before Dose 2), Day 15 (7 days after Dose 2), Day 30 (immediately before Dose 3), Day 37 (7 days after Dose 3), Month 2 (1 month after Dose 3), Month 4 (3 months after Dose 3), and Month 7 (6 months after Dose 3).

Both toxin A- and toxin B-specific neutralizing antibody levels were measured.

Toxin A- and Toxin B-Specific Neutralizing Antibody Geometric Mean Concentrations—For the month regimen, Month 7 (1 month after Dose 3) was specified as the primary time point, as it reflected the immune response after the third dose of the vaccine. Overall, toxin A- and toxin B-specific neutralizing antibody GMCs increased after Dose 2 but were highest after Dose 3 (Month 7) for both the 100- and 200-μg dose groups. Toxin A- and toxin B-specific neutralizing antibody GMCs were higher in the 200-μg dose group compared to the 100-μg dose group at Month 7. Toxin A- and toxin B-specific neutralizing antibody GMCs decreased from Month 7 until Month 12 for both dose levels. At Month 12, GMCs remained above threshold for both toxin A- and toxin B-specific neutralizing antibody for the 200-μg dose group and also for toxin A-specific neutralizing antibody for the 100-μg dose group. Toxin A-specific neutralizing antibody GMCs in the 200-μg dose group increased after Dose 2 (Day 37) to 347.17 neutralizing units/mL, and to 125.46 neutralizing units/mL in the 100-μg dose group at Day 37. Toxin A-specific neutralizing antibody GMCs in both the 100- and 200-μg dose groups decreased by Month 6 (before Dose 3) to 88.89 neutralizing units/mL and 148.44 neutralizing units/mL, respectively. After Dose 3, toxin A-specific neutralizing antibody GMCs again increased for both the 100- and 200-μg dose groups, and at Month 7 were numerically higher in the 200-μg dose group (1692.38 neutralizing units/mL) when compared to the 100-μg dose group (1137.50 neutralizing units/mL). Toxin A-specific neutralizing antibody GMCs then decreased until Month 12 (6 months after Dose 3), although both the 100- and 200-μg dose groups remained generally high (248.97 neutralizing units/mL and 587.46 neutralizing units/mL, respectively) compared to GMCs at Month 6. Toxin B-specific neutralizing antibody GMCs in both the 100- and 200-μg dose groups increased after Dose 2 (Day 37) to 337.54 neutralizing units/mL and 901.16 neutralizing units/mL, respectively. Toxin B-specific neutralizing antibody GMCs decreased by Month 6 (before Dose 3) to 274.13 neutralizing units/mL and 689.74 neutralizing units/mL, respectively. After Dose 3 (Day 187), toxin B-specific neutralizing antibody GMCs again increased for both the 100- and 200-μg dose group, and at Month 7 toxin B-specific neutralizing antibody GMCs were numerically higher in the 200-μg dose group (13756.54 neutralizing units/mL) when compared to the 100-μg dose group (7903.68 neutralizing units/mL). At Month 12, toxin B-specific neutralizing antibody GMCs decreased, but remained generally high for the 200-μg dose group (6298.18 neutralizing units/mL) but not the 100-μg dose group (1887.24 neutralizing units/mL) compared to GMCs at Month 6. Toxin A- and toxin B-specific neutralizing antibody GMCs remained unchanged from baseline to Month 12 in the placebo group. For the month regimen, toxin A- and toxin B-specific neutralizing antibody GMCs were basically similar among subjects aged 65 to 69 years and 70 to 74 years in the evaluable immunogenicity population.

In the month regimen, toxin A- and toxin B-specific neutralizing antibody Geometric Mean Fold Rises (GMFRs) from baseline were calculated at each available postbaseline time point: Day 15 (14 days after Dose 1), Day 30 (immediately before Dose 2), Day 37 (7 days after Dose 2), Month 2 (1 month after Dose 2), Month 6 (immediately before Dose 3), Day 187 (7 days after Dose 3), Month 7 (1 month after Dose 3, primary time point), and Month 12 (6 months after Dose 3).

A toxin A-specific neutralizing antibody GMFR increase was observed in the 200-μg dose group after Dose 2 (Day 37; 4.26), but decreased to 1.82 by Month 6. After Dose 3, there was a further increase in toxin A-specific neutralizing antibody GMFRs for both the 100- and 200-μg dose groups at Day 187 (7.42 and 16.07, respectively), and a further increase at Month 7 (12.58 and 20.77, respectively). By Month 12, toxin A-specific neutralizing antibody GMFRs decreased to 2.70 and 7.20 in the 100- and 200-μg dose groups, respectively. Toxin A-specific neutralizing antibody GMFRs in the 200-μg dose group were consistently higher than those of the 100-μg dose group at each blood sampling time point. Toxin A-specific neutralizing antibody GMFRs remained unchanged in the placebo group throughout the study.

Increases in toxin B-specific neutralizing antibody GMFRs were observed for the 100- and 200-μg dose groups after Dose 1 (Day 15; 2.81 and 5.21, respectively) and Dose 2 (Day 37; 2.53 and 5.35, respectively), with slight decreases observed by Month 6 (2.06 and 4.10, respectively). A further increase in toxin B-specific neutralizing antibody GMFRs was observed after Dose 3 for both the 100- and 200-μg dose groups at Day 187 (32.00 and 54.81, respectively), and a further increase at Month 7 (59.28 and 81.71, respectively). By Month 12, toxin B-specific neutralizing antibody GMFRs decreased to 14.01 and 36.90 for the 100- and 200-μg dose groups, respectively. Toxin B-specific neutralizing antibody GMFRs in the 200-μg dose group were consistently higher than those of the 100-μg dose group at each blood sampling time point. Toxin B-specific neutralizing antibody GMFRs remained unchanged in the placebo group throughout the study.

The proportions of subjects in the month regimen who achieved toxin A-, toxin B-, and both toxin A- and B-specific neutralizing antibody levels above the specified threshold values (219 neutralizing units/mL [toxin A] and 2586 neutralizing units/mL [toxin B]) were assessed for the evaluable immunogenicity population.

Overall, the proportions of subjects who achieved toxin A- and toxin B-specific neutralizing antibody levels above the threshold were higher in the 200-μg dose group when compared to the 100-μg dose group across all blood sampling time points. For both dose groups, the proportions of subjects with toxin A-, toxin B-, and both toxin A- and toxin B-specific neutralizing antibody levels above the threshold increased after Dose 2 and were highest after Dose 3 (Month 7).

For toxin A-specific neutralizing antibody, after Dose 3 (Day 187), 78.3% (18/23, 95% CI: 56.3, 92.5) of subjects in the 100-μg dose group and 95.7% (22/23, 95% CI: 78.1, 99.9) of subjects in the 200-μg dose group achieved toxin A-specific neutralizing antibody levels above the threshold, compared to no subjects in the placebo group. At Month 7, 91.3% (21/23, 95% CI: 72.0, 98.9) of subjects in the 100-μg dose group and 100.0% (23/23, 95% CI: 85.2, 100.0) subjects in the 200-μg dose group achieved toxin A-specific neutralizing antibody levels above the threshold, compared to no subjects in the placebo group. The proportions of subjects above the threshold for toxin A-specific neutralizing antibody levels remained high at Month 12 for the 200-μg dose group (95.5% [21/22] of subjects, 95% CI: 77.2, 99.9), but declined in the 100-dose group (55.0% [9/20] of subjects, 95% CI: 31.5, 76.9). No subjects in the placebo group had toxin A-specific neutralizing antibody levels above the threshold at Month 12.

For toxin B-specific neutralizing antibody, after Dose 3 (Day 187), 52.2% (12/23, 95% CI: 30.6, 73.2) of subjects in the 100-μg dose group and 91.3% (21/23, 95% CI: 72.0, 98.9) subjects in the 200-μg dose group achieved toxin B-specific neutralizing antibody levels above the threshold, compared to no subjects in the placebo group. At Month 7, 91.3% (21/23, 95% CI: 72.0, 98.9) of subjects in the 100-μg dose group and 100.0% (23/23, 95% CI: 85.2, 100.0) of subjects in the 200-μg dose group achieved toxin B-specific neutralizing antibody levels above the threshold, compared to no subjects in the placebo group. The proportions of subjects above the threshold for toxin B-specific neutralizing antibody levels remained high at Month 12 for the 200-μg dose group (81.8% [18/22] of subjects, 95% CI: 59.7, 94.8), but declined in the 100-dose group (45.0% [11/20] of subjects, 95% CI: 23.1, 68.5). No subjects in the placebo group had toxin B-specific neutralizing antibody levels above threshold at Month 12.

For the month regimen, the proportions of subjects achieving defined fold rises (≥4-fold, ≥8-fold, ≥16-fold, and ≥32-fold) from baseline in toxin A-, toxin B-, and both toxin A- and toxin B-specific neutralizing antibody levels were assessed for the evaluable immunogenicity population. Overall, a greater proportion of subjects achieving defined fold rises from baseline toxin A-specific neutralizing antibody levels was observed in the 200-μg dose group compared to the 100-μg dose group. An increase in the proportions of subjects achieving ≥32-fold rise from baseline in both the 100- and 200-μg dose groups was observed after Dose 3 (Day 187). By Month 7, 69.6% (16/23) of subjects in the 100-μg dose group and 91.3% (21/23) of subjects in the 200-μg dose group achieved an ≥8-fold rise from baseline. Further at Month 7, 69.6% (16/23) of subjects in the 200-μg dose group also achieved a ≥16-fold rise from baseline. Toxin A-specific neutralizing antibody fold rises from baseline in the placebo group remained unchanged at each time point. The proportions of subjects achieving defined fold rises from baseline toxin A-specific neutralizing antibody levels declined at Month 12 compared to Month 7, with 5.0% (1/20) of subjects in the 100-μg dose group and 13.6% (3/22) of subjects in the 200-μg dose group achieving a ≥16-fold rise from baseline.

Overall, a greater proportion of subjects achieving defined fold rises from baseline toxin B-specific neutralizing antibody levels was observed in the 200-μg dose group compared to the 100-μg dose group. An increase in the proportions of subjects achieving ≥32-fold rises from baseline in both the 100- and 200-μg dose groups was observed after Dose 3 (Day 187). By Month 7, 73.9% (17/23) of subjects in the 100-μg dose group and 82.6% (19/23) of subjects in the 200-μg dose group achieved a ≥32-fold rise from baseline. Toxin B-specific neutralizing antibody fold rises from baseline in the placebo group remained unchanged at each time point. The proportions of subjects achieving defined fold rises from baseline toxin B-specific neutralizing antibody levels declined at Month 12 compared to Month 7, with 25.0% (5/20) of subjects in the 100-μg dose group and 45.5% (10/22) of subjects in the 200-μg dose group achieving a ≥32-fold rise from baseline.

An increase in the proportions of subjects achieving ≥8-fold rises from baseline for both toxin A- and B-specific neutralizing antibody levels in both the 100- and 200-μg dose groups was observed after Dose 3 (Day 187). By Month 7, 69.6% (16/23) of subjects in the 100-μg dose group and 87.0% (20/23) of subjects in the 200-μg dose group achieved an ≥8-fold rise from baseline. Further at Month 7, 65.2% (15/23) of subjects in the 200-μg dose group achieved a ≥16-fold rise from baseline. Both toxin A- and B-specific neutralizing antibody fold rises from baseline in the placebo group remained unchanged at each time point.

The proportions of subjects with toxin A- and B-specific neutralizing antibody levels LLOQ for the month regimen were assessed. Overall, a low proportion of subjects in both the 100- and 200-μg dose groups had either toxin A- or toxin B-specific neutralizing antibody levels≤LLOQ at Month 7 and Month 12. The majority of subjects in the placebo group had toxin A-, toxin B-, and toxin A- and toxin B-specific neutralizing antibody levels≤LLOQ at all time points.

For the day regimen, Day 37 (7 days after Dose 3) was specified as the primary time point, as it reflected the immune response after the third dose of the vaccine; however, due to the limited number of enrolled subjects resulting from the decision to discontinue subsequent dosing following the stopping rule, inferences could not be made at Day 30 and beyond. Toxin A-specific neutralizing antibody GMCs for the 100- and 200-μg dose groups increased after Dose 1 and Dose 2, but to a greater extent in the 200-μg dose group at Day 15 (515.67 neutralizing units/mL) and Day 30 (673.00 neutralizing units/mL) when compared to the 100-μg dose group (79.00 neutralizing units/mL at Day 15 and 116.21 neutralizing units/mL at Day 30). Toxin A-specific neutralizing antibody GMCs remained unchanged throughout the study in the placebo group. Similar to toxin A, toxin B-specific neutralizing antibody GMCs for the 100- and 200-μg dose groups increased after Dose 1 and Dose 2, but to a greater extent in the 200-μg dose group at Day 15 (3531.69 neutralizing units/mL) and Day 30 (4666.51 neutralizing units/mL) when compared to the 100-μg dose group (692.98 neutralizing units/mL and 682.09 neutralizing units/mL). Toxin B-specific neutralizing antibody GMCs remained unchanged throughout the study in the placebo group.

In the day regimen, toxin A- and toxin B-specific neutralizing antibody GMFRs from baseline were calculated at each available postbaseline time point: Day 8 (immediately before Dose 2), Day 15 (7 days after Dose 2), Day 30 (immediately before Dose 3), Day 37 (7 days after Dose 3, primary time point), Month 2 (1 month after Dose 3), Month 4 (3 months after Dose 3), and Month 7 (6 months after Dose 3). Due to the limited number of subjects caused by the decision to discontinue subsequent dosing following the stopping rule, inferences could not be made at Day 30 and beyond. A toxin A-specific neutralizing antibody GMFR increase was observed in the 200-μg dose group after Dose 2 on Day 15 (6.11), and a further increase at Day 30 (7.79).

An increase in toxin A-specific neutralizing antibody GMFRs was observed after Dose 2 in the 100-μg dose group at Day 30 (1.47), but overall was lower than the GMFRs observed in the 200-μg dose group at all time points. Toxin A-specific neutralizing antibody GMFRs remained unchanged in the placebo group at all time points.

Increases in toxin B-specific neutralizing antibody GMFRs were observed in both the 100- and 200-μg dose groups after Dose 1 (Day 8: 1.41 and 1.78, respectively), with further increases observed in both dose groups at Day 15 (4.37 and 9.01, respectively) and Day 30 (4.59 and 10.63, respectively). Toxin B-specific neutralizing antibody GMFRs were consistently higher in the 200-μg dose group when compared to those in the 100-μg dose group. Toxin B-specific neutralizing antibody GMFRs remained unchanged in the placebo group at all time points.

In the day regimen, the proportions of subjects in the mITT population who achieved toxin A-, toxin B-, and both toxin A- and B-specific neutralizing antibody levels above the specified threshold values (219 neutralizing units/mL [toxin A] and 2586 neutralizing units/mL [toxin B]) were assessed. By Day 30 in the day regimen, a higher percentage of subjects in the 200-μg dose group (62.5% [5/8] of subjects, 95% CI: 24.5, 91.5), compared to the 100-μg dose group (20.0% [1/5] of subjects, 95% CI: 0.5, 71.6), achieved toxin A-specific neutralizing antibody levels above the threshold.

Similarly, for toxin B-specific neutralizing antibody, at Day 30, a higher percentage of subjects in the 200-μg dose group (62.5% [5/8] of subjects, 95% CI: 24.5, 91.5), compared to the 100-μg dose group (20.0% [1/5] of subjects, 95% CI: 0.5, 71.6), achieved toxin B-specific neutralizing antibody levels above the threshold.

For both toxin A and toxin B-specific neutralizing antibody, half of the subjects achieved levels above the threshold by Day 30 in the 200-μg dose group (50.0% [4/8] of subjects, 95% CI: 15.7, 84.3), while none of the subjects achieved levels above the threshold for both toxin A and toxin B in the 100-μg dose group (0.0% [0/5] of subjects).

In the day regimen, the proportions of subjects achieving defined fold rises (≥4-fold, ≥8-fold, ≥16-fold, and ≥32-fold) from baseline in toxin A-, toxin B-, and both toxin A- and toxin B-specific neutralizing antibody levels were assessed. Overall, a greater proportion of subjects in the day regimen achieving defined fold rises from baseline toxin A-specific neutralizing antibody levels was observed in the 200-μg dose group compared to the 100-μg dose group. No subjects in the 100-μg dose group achieved a ≥4-fold or higher rise in toxin A-specific neutralizing antibody levels through to Day 30. There was an increase in the proportions of subjects in the 200-μg dose group achieving ≥32-fold rises from baseline after Dose 2 (Day 15), which continued through to Day 30 (25.0% [2/8] of subjects in the 200-μg dose group achieved a ≥32-fold rise from baseline). Toxin A-specific neutralizing antibody fold rises from baseline in the placebo group remained unchanged at each time point.

Similar to toxin A-specific neutralizing antibody, a greater proportion of subjects in the day regimen achieving defined fold rises from baseline toxin B-specific neutralizing antibody levels was observed in the 200-μg dose group compared to the 100-μg dose group. There was an increase in the proportions of subjects achieving ≥32-fold rises from baseline in both the 100- and 200-μg dose groups after Dose 2 (Day 15), which continued through to Day 30. At Day 30, only 1 of the 5 subjects in the 100-μg dose group achieved any of the defined fold rises. Conversely, 62.5% [5/8] of subjects in the 200-μg dose group achieved a ≥16-fold rise at Day 30. Toxin B-specific neutralizing antibody fold rises from baseline in the placebo group remained unchanged at each time point.

No subjects in the 100-μg dose group achieved a ≥4-fold or higher rise in both toxin A- and toxin B-specific neutralizing antibody levels up to and including Day 30. There was an increase in the proportions of subjects achieving ≥32-fold rises from baseline for both toxins A and B in the 200-μg dose group after Dose 2 (Day 15), which continued through to Day 30 (12.5% [1/8] of subjects in the 200-μg dose group achieved a ≥32-fold rise from baseline). Toxin A- and toxin B-specific neutralizing antibody fold rises from baseline in the placebo group remained unchanged at each time point.

The proportions of subjects with toxin A- and toxin B-specific neutralizing antibody levels<LLOQ for the day regimen were assessed. Subjects in both the 100- and 200-μg dose groups had toxin A-specific neutralizing antibody levels<LLOQ after Dose 2 at Day 15 (100.0% [11/11] of subjects and 54.5% [6/11] of subjects, respectively), and was reduced at Day 30 (60.0% [3/5] of subjects and 25.0% [2/8] of subjects, respectively). By Day 30, subjects in both the 100- and 200-μg dose groups had toxin B-specific neutralizing antibody levels<LLOQ (40.0% [2/5] of subjects and 37.5% [3/8] of subjects, respectively) and both toxin A- and toxin B-specific neutralizing antibody levels<LLOQ (20.0% [1/5] of subjects and 12.5% [1/8] of subjects, respectively).

Conclusions. Immunogenicity endpoints were considered secondary endpoints in this study. Immunogenicity endpoints included toxin A- and toxin B-specific neutralizing antibody GMCs and GMFRs from baseline, and the proportions of subjects with toxin A-, toxin B-, or both toxin A- and toxin B-specific neutralizing antibody levels above the threshold, and the proportions with ≥4-fold, ≥8-fold, ≥16-fold, and ≥32-fold rises from baseline. As a result of stopping the day regimen, only a limited amount of immunogenicity data was available for this cohort after 30 days. Therefore, the following conclusions were made from data obtained from the month regimen. Overall, GMCs for toxin A- and toxin B-specific neutralizing antibody levels increased after Dose 1, were maximal at Month 7, and declined until Month 12 in both the 100- and 200-μg dose groups. At Month 7, both toxin A- and toxin B-specific neutralizing antibody GMCs were numerically higher in the 200-μg dose group (1692.38 and 13756.54 neutralizing units/mL, respectively) when compared to the 100-μg dose group (1137.50 and 7903.68 neutralizing units/mL, respectively). By Month 12, toxin A- and toxin B-specific neutralizing antibody GMCs declined, but remained high in the 200-μg dose group (587.46 and 6298.18 neutralizing units/mL, respectively) compared to GMCs at Month 6. Toxin A-specific neutralizing antibody GMFRs in the 200-μg dose group were consistently higher than those of the 100-μg dose group at each blood sampling time point. After Dose 3, increases in GMFRs from baseline were observed in both the 100- and 200-μg dose groups. Toxin A and toxin B-specific neutralizing antibody GMFRs were highest at Month 7 and were numerically higher in the 200-μg dose group (20.77 and 81.71, respectively) when compared to the 100-μg dose group (12.58 and 59.28, respectively). Toxin A- and toxin B-specific neutralizing antibody GMFRs then declined by Month 12 in both vaccine groups. The proportions of subjects who achieved toxin A- and toxin B-specific neutralizing antibody levels above the threshold were generally higher in the 200-μg dose group when compared to the 100-μg dose group across all blood sampling time points. By Month 7, 100.0% of subjects in the 200-μg dose group achieved toxin A- and toxin B-specific neutralizing antibody levels above the threshold, while more than 90% of subjects in the 100-μg dose group achieved levels above the threshold. The proportions of subjects above the threshold for toxin A- and toxin B-specific neutralizing antibody levels remained high at Month 12 for the 200-μg dose group (95.5% and 81.8%, respectively), but declined in the 100-dose group (55.0% and 45.0%, respectively). No subjects in the placebo group had toxin A- or toxin B-specific neutralizing antibody levels above the threshold at Month 12. For toxin A-specific neutralizing antibody, 69.6% (16/23) of subjects in the 100-μg dose group and 91.3% (21/23) of subjects in the 200-μg dose group achieved a ≥8-fold rise from baseline by Month 7, with a majority of subjects (69.6%; 16/23) in the 200-μg dose group also achieving a ≥16-fold rise from baseline by Month 7. The proportions of subjects achieving defined fold rises from baseline toxin A-specific neutralizing antibody levels declined at Month 12 compared to Month 7, with 5.0% (1/20) of subjects in the 100-μg dose group and 13.6% (3/22) of subjects in the 200-μg dose group achieving a ≥16-fold rise from baseline. For toxin B-specific neutralizing antibody, the majority of subjects in both the 100- and 200-μg dose groups achieved a ≥32-fold rise from baseline (73.9% [17/23] of subjects and 82.6% [19/23] of subjects, respectively) by Month 7. The proportions of subjects achieving defined fold rises from baseline toxin B-specific neutralizing antibody levels declined at Month 12 compared to Month 7, with 25.0% (5/20) of subjects in the 100-μg dose group and 45.5% (10/22) of subjects in the 200-μg dose group achieving a ≥32-fold rise from baseline. Due to very small numbers of seropositive subjects at baseline in the month and day regimens at baseline across the 100-μg, 200-μg, and placebo doses, the effects of baseline serostatus on GMCs were not able to be considered. As evident in the month regimen RCDCs, the 200-μg dose group had higher toxin A- and toxin B-specific neutralizing antibody concentrations when compared to the 100-μg dose group at Month 7, and in general across all time points. Taken together, these results demonstrate that, in the month regimen, peak immunogenicity occurs approximately 1 month after Dose 3 (Month 7) of the C. difficile vaccine. Additionally, the 200-μg dose induces a higher and more persistent antibody response compared to the 100-μg dose, as assessed by toxin A- and toxin B-specific neutralizing antibody GMCs and GMFRs.

### Example 10

### Antibodies Induced by Immunogenic Compositions are Capable of Neutralizing Toxins from Various C. difficile Strains

Culture supernatants containing secreted toxins from the various strains were tested in an in vitro neutralization assay using sera from non-human primates immunized with the immunogenic composition to determine the coverage of the immunogenic composition and to determine the ability of the immunogenic composition to protect against diverse toxins from circulating clinical strains.

IMR-90 cells were used to test the neutralization of toxins expressed from various C. difficile strains. Neutralization titers of test samples are calculated based on a Reference standard. The assay LLOQ: Txd A=158.0 U/ml, Txd B=249.5 U/ml. The results in Table 5 show that sera from immunized non-human primates were able to neutralize C. difficile toxins from each of the respective culture supernatants in the neutralization assay.

TABLE 5
Description of Clostridium difficile Strains: Epidemiological Markers, Toxin Genotype, Sequence
Identity with Vaccine Antigen, and the Ability of NHP Antitoxin to Neutralize Toxins

| | | | | | | | Ability of NHP |
|---|---|---|---|---|---|---|---|
| | | | | | Toxin Variant | | Antitoxin to |
| | | | | | (% Identity to | | Neutralize C |
| C difficile | Source of | PFGE | | Toxino- | Vaccine Antigen^{a}) | | difficile |
| Strain | Strain | Type | Ribotype | type | TcdB | TcdA | toxins |
| PFECD0003 | Europe | NAP6 | 002 | 0 | TcdB_016 | TcdA_017 | + |
| | | | | | (99.7) | (99.7) | |
| PFECD0005 | Europe | NAP10 | 003 | I | TcdB_010 | TcdA_018 | + |
| | | | | | (99.8) | (99.7) | |
| PFECD0046 | Europe | NAPCR1 | 004 | 0 | TcdB_001 | TcdA_006 | + |
| | | | | | (99.9) | (99.8) | |
| PFECD0078 | Europe | NA | 012 | 0 | TcdB_001 | TcdA_001 | + |
| | | | | | (99.9) | (99.9) | |
| PFECD0008 | Canada | NAP12 | 015 | 0 | TcdB_005 | TcdA_014 | + |
| | | | | | (99.8) | (99.6) | |
| PFECD0010 | US | NAP9 | 017 | VIII | TcdB_003 | Truncated^{c} | + |
| | | | | | (93.7) | | |
| PFECD0011 | Europe | NAP4 | 020 | 0 | TcdB_007 | TcdA_003 | + |
| | | | | | (99.8) | (99.7) | |
| PFECD0012 | Europe | NA | 023 | IV | TcdB_006 | TcdA_019 | + |
| | | | | | (98.1) | (98.3) | |
| PFECD0013 | Europe | NAP1 | 027 | III | TcdB_012 | TcdA_010 | + |
| | | | | | (99.8) | (99.8) | |
| PFECD0015 | US | NAP1 | 027 | III | TcdB_002 | TcdA_007 | + |
| | | | | | (92.1) | (98.1) | |
| PFECD0016 | Europe | NA | 029 | 0 | TcdB_006 | TcdA_019 | + |
| | | | | | (98.1) | (98.3) | |
| PFECD0017 | Europe | NA | 046 | 0 | TcdB_008 | TcdA_004 | + |
| | | | | | (99.8) | (99.7) | |
| PFECD0019 | US | NAP3 | 053 | 0 | TcdB_008 | TcdA_012 | + |
| | | | | | (99.8) | (99.7) | |
| PFECD0021 | Europe | NA | 059 | IV | TcdB_015 | TcdA_011 | + |
| | | | | | (99.7) | (99.7) | |
| PFECD0022 | Europe | NA | 070 | XIII | TcdB_016 | TcdA_017 | + |
| | | | | | (99.7) | (99.7) | |
| PFECD0023 | US | NAP10 | 070 | 0 | TcdB_012 | TcdA_015 | + |
| | | | | | (99.8) | (99.8) | |
| PFECD0024 | Europe | NA | 075 | III | TcdB_012 | TcdA_010 | + |
| | | | | | (99.8) | (99.8) | |
| PFECD0027 | Europe | NA | 078 | 0 | TcdB_004 | TcdA_013 | + |
| | | | | | (95.9) | (97.9) | |
| PFECD0030 | US | NAP7 | 078 | V | TcdB_004 | TcdA_013 | + |
| | | | | | (95.9) | (97.9) | |
| PFECD0031 | Europe | NA | 081 | 0 | TcdB_013 | TcdA_009 | + |
| | | | | | (99.8) | (99.7) | |
| PFECD0032 | Europe | NA | 087 | 0 | TcdB_001 | TcdA_009 | + |
| | | | | | (99.9) | (99.7) | |
| PFECD0035 | US | NAP11 | 106 | 0 | TcdB_009 | TcdA_002 | + |
| | | | | | (99.8) | (99.7) | |
| PFECD0036 | Europe | NA | 117 | 0 | TcdB_001 | TcdA_005 | + |
| | | | | | (99.9) | (99.9) | |
| PFECD0037 | Europe | NA | 126 | NA | TcdB_016 | TcdA_017 | + |
| | | | | | (99.7) | (99.7) | |
| PFECD0039 | Canada | NAP7 | 126 | V | TcdB_011 | TcdA_016 | + |
| | | | | | (86.9) | (98.3) | |
| PFECD0040 | Europe | NA | 131 | NA | TcdB_016 | TcdA_017 | + |
| | | | | | (99.7) | (99.7) | |
| PFECD0041 | US | NAP4 | 154 | 0 | TcdB_014 | TcdA_010 | + |
| | | | | | (99.8) | (99.8) | |
| PFECD0043 | US | NAP11 | NA^{b} | 0 | TcdB_008 | TcdA_008 | + |
| | | | | | (99.8) | (99.7) | |
| PFECD0049 | US | NAP2 | NA^{b} | 0 | TcdB_012 | TcdA_010 | + |
| | | | | | (99.8) | (99.8) | |

### Example 11

### Diagnostic Assays in Support of a Phase 3 C. difficile Vaccine Efficacy Study

Clover (B5091007) is a multinational pivotal Phase 3 study evaluating the efficacy, safety and tolerability of a toxoid-based Clostridium (C) difficile vaccine in subjects 50 years of age or older who have an increased risk of CDI. Involving approximately 400 investigational sites in 23 countries, the study is targeted to enroll nearly 16,000 subjects. The primary objective of this Phase 3 study will be to demonstrate vaccine efficacy in reducing the incidence of a first primary episode of CDI based on both clinical and laboratory diagnostic criteria. Evaluating the efficacy of the vaccine necessitates tracking when subjects experience diarrhea, collecting stool samples when they do, having those samples shipped to a central laboratory in temperature-controlled conditions and testing them for presence of C. difficile and the toxin(s) that cause the disease. To ensure accurate laboratory diagnosis of CDI, a two-step algorithm is used for testing stool samples. This algorithm is based on detection of C. difficile strains/spores harboring the toxin B gene by PCR followed by detection of free toxins (A and B) using a proprietary toxin detection test (CCNA). This approach was chosen, as epidemiological studies clearly demonstrated that the detection of free toxins was a better predictor of CDI disease than PCR alone.i, ii In addition, a two-step testing algorithm had already been recommended in the EU by the European Society of Clinical Microbiology and Infectious Diseases and in the US by the Infectious Diseases Society of America and the Society for Healthcare Epidemiology of America. Highlights of the operational/executional challenges of conducting this study and the PCR qualification and CCNA validation/clinical validation, are described. Both assays met all prespecified acceptance criteria and are suitable for their intended use as diagnostics in C. difficile vaccine efficacy and epidemiology studies.

Qualification of the CEPHEID XPERT® C. difficile/Epi PCR test Validation and Clinical Validation of the CCNA

The XPERT® C. difficile/Epi PCR test and CCNA are sensitive, robust, and reproducible. Assay qualification and validation of precision, linearity, accuracy and/or specificity confirmed the XPERT® C. difficile/Epi PCR test and CCNA are suitable for their intended purpose. Each assay was tested against true CDI positive and negative stool samples and evaluated for clinical accuracy. Both the XPERT® C. difficile/Epi PCR test and CCNA were clinically accurate by classifying positive and negative samples appropriately.

Specificity: 50 non toxigenic and non C. difficile strains were evaluated with the XPERT® C. difficile/Epi PCR. No cross reaction was observed, which corresponds to an analytical specificity of 100%.

Sensitivity: The limit of detection (LOD) of 10 C. difficile strains spiked in stool was determined. The LOD for the 10 strains ranged from 344 to 2175 colony forming units (CFU).

Precision: 9 input samples were tested by 2 analysts, using 3 lots of test reagent over 10 test days. Sample variability, under these conditions, was <2% Relative Standard Deviations.

Accuracy: The agreement between the measured value and the reference value at 10-fold increases in concentration is indicated by the slope=−3.3 Ct.

The CEPHEID XPERT® C. difficile/Epi PCR test correctly identified all 93 CDI case samples in clinical validation; excellent CCNA precision was observed during assay validation; the CCNA demonstrated 100% clinical specificity when compared to a reference method; and the CCNA demonstrated 95.7% clinical sensitivity when compared to another reference method.

Conclusion: A 2-step algorithm is used for laboratory confirmation of CDI based on the detection of the C. difficile organism followed by the detection of toxins. Prior to being used in CLOVER, both the XPERT® C. difficile/Epi PCR test and CCNA were qualified and/or validated and both demonstrated excellent analytical specificity, sensitivity, and precision. During clinical validation, both diagnostic assays were clinically specific and sensitive in the detection of presumed CDI cases. Each assay used in conjunction with a reliable clinical sample specimen collection strategy are currently being used in a C. difficile vaccine phase 3 clinical trial.

The following clauses describe additional embodiments of the invention:
C1.A method for eliciting an immune response in a human against a Clostridium difficile infection, the method comprising administering to the human an effective dose of a composition, which comprises a C. difficile toxoid, wherein the composition is administered at least two times.
C2.The method according to clause C1, wherein the second administration is at least 7 days after the first administration and the third administration is about 30 days after the first administration.
C3.The method according to Clause C1, wherein the third administration is about 180 days after the first administration.
C4.The method according to Clause C1, wherein the composition is administered at least three times.
C5.The method according to Clause C2, wherein the second administration is about 30 days after the first administration and the third administration is about 180 days after the first or second administration.
C6.The method according to Clause C2, wherein the third administration is at least 180 days after the first administration.
C7.The method according to Clause C1, wherein the immune response elicited comprises an anti-toxin A neutralizing monoclonal antibody.
C8.The method according to Clause C1, wherein the immune response elicited comprises an anti-toxin B neutralizing monoclonal antibody.
C9.The method according to Clause C1, wherein the immune response elicited comprises an anti-toxin A neutralizing monoclonal antibody and an anti-toxin B neutralizing monoclonal antibody, wherein the concentration of neutralizing monoclonal antibody is at least 10 μg/mL.
C10. The method according to Clause C1, wherein the composition comprises a C. difficile toxoid A and/or a C. difficile toxoid B, each having a purity of at least 90% or greater.
C11. The method according to Clause C1, wherein the composition comprises a C. difficile toxoid A and/or a C. difficile toxoid B, in a ratio of about 3:1 to about 1:1.
C12. The method according to Clause C1 wherein the composition comprises a C. difficile toxoid A and/or a C. difficile toxoid B, in a ratio of 1:1.
C13. The method according to Clause C1, wherein the composition comprises an adjuvant.
C14. The method according to Clause C1, wherein the composition comprises an aluminum adjuvant.
C15. The method according to Clause C1, wherein the immune response against C. difficile toxin A and/or toxin B is sustained for at least about 60 days.
C16. The method according to Clause C1, wherein the immune response against C. difficile toxin A and/or toxin B is sustained for at least about 180 days.
C17. The method according to Clause C1, wherein the immune response against C. difficile toxin A and/or toxin B is sustained for at least about 365 days.
C18. The method according to Clause C1, wherein the immune response against C. difficile toxin A and/or toxin B is sustained for at least about 540 days.
C19. The method according to Clause C1, wherein the second administration is at least 7 days after the first administration and the third administration is at least 30 days after the first administration.
C20. The method according to Clause C1, wherein the third administration is at least 30 days after the first administration.
C21. The method according to Clause C1, wherein the second administration is at least 7 days after the first administration and the third administration is at least 180 days after the first or second administration.
C22. The method according to Clause C1, wherein the third administration is at least 180 days after the first administration.
C23. The method according to Clause C1, wherein the composition comprises a C. difficile toxoid A and/or a C. difficile toxoid B, each having a purity of at least 90% or greater.
C24. The method according to Clause C1, wherein the composition comprises a C. difficile toxoid A and/or a C. difficile toxoid B, in a ratio of about 3:1 to about 1:1.
C25. The method according to Clause C1, wherein the composition comprises a C. difficile toxoid A and/or a C. difficile toxoid B, in a ratio of 1:1.
C26. The method according to Clause C1, wherein the composition comprises an adjuvant.
C27. The method according to Clause C1, wherein the composition comprises an aluminum adjuvant.
C28. The method according to Clause C1, wherein the immune response against C. difficile toxin A and/or toxin B is sustained for at least about 60 days.
C29. The method according to Clause C1, wherein the immune response against C. difficile toxin A and/or toxin B is sustained for at least about 180 days.
C30. The method according to Clause C1, wherein the immune response against C. difficile toxin A and/or toxin B is sustained for at least about 365 days.
C31. The method according to Clause C1, wherein the C. difficile toxoid A is bound to aluminum adjuvant.
C32. The method according to Clause C1, wherein the C. difficile toxoid B is bound to aluminum adjuvant.
C33. The method according to Clause C1, wherein the C. difficile toxoid A and/or a C. difficile toxoid B are lyophilized.
C34. The method according to Clause C1, wherein the composition induces a toxin A-specific neutralizing antibody concentration that is at least 2-fold higher in the human after receiving the first dose than a toxin A-specific neutralizing antibody concentration in the human prior to receiving the first dose, when measured under identical conditions in a cytotoxicity assay.
C35. The method according to Clause C1, wherein the composition induces a toxin A-specific neutralizing antibody concentration that is at least 4-fold higher in the human after receiving the first dose than a toxin A-specific neutralizing antibody concentration in the human prior to receiving the first dose, when measured under identical conditions in a cytotoxicity assay.
C36. The method according to Clause C1, wherein the composition induces a toxin A-specific neutralizing antibody concentration that is at least 8-fold higher in the human after receiving the first dose than a toxin A-specific neutralizing antibody concentration in the human prior to receiving the first dose, when measured under identical conditions in a cytotoxicity assay.
C37. The method according to Clause C1, wherein the composition induces a toxin A-specific neutralizing antibody concentration that is at least 16-fold higher in the human after receiving the first dose than a toxin A-specific neutralizing antibody concentration in the human prior to receiving the first dose, when measured under identical conditions in a cytotoxicity assay.
C38. The method according to Clause C1, wherein the composition induces a toxin A-specific neutralizing antibody concentration that is at least 32-fold higher in the human after receiving the first dose than a toxin A-specific neutralizing antibody concentration in the human prior to receiving the first dose, when measured under identical conditions in a cytotoxicity assay.
C39. The method according to Clause C1, wherein the composition induces a toxin B-specific neutralizing antibody concentration that is at least 2-fold higher in the human after receiving the first dose than a toxin B-specific neutralizing antibody concentration in the human prior to receiving the first dose, when measured under identical conditions in a cytotoxicity assay.
C40. The method according to Clause C1, wherein the composition induces a toxin B-specific neutralizing antibody concentration that is at least 4-fold higher in the human after receiving the first dose than a toxin B-specific neutralizing antibody concentration in the human prior to receiving the first dose, when measured under identical conditions in a cytotoxicity assay.
C41. The method according to Clause C1, wherein the composition induces a toxin B-specific neutralizing antibody concentration that is at least 8-fold higher in the human after receiving the first dose than a toxin B-specific neutralizing antibody concentration in the human prior to receiving the first dose, when measured under identical conditions in a cytotoxicity assay.
C42. The method according to Clause C1, wherein the composition induces a toxin B-specific neutralizing antibody concentration that is at least 16-fold higher in the human after receiving the first dose than a toxin B-specific neutralizing antibody concentration in the human prior to receiving the first dose, when measured under identical conditions in a cytotoxicity assay.
C43. The method according to Clause C1, wherein the composition induces a toxin B-specific neutralizing antibody concentration that is at least 32-fold higher in the human after receiving the first dose than a toxin B-specific neutralizing antibody concentration in the human prior to receiving the first dose, when measured under identical conditions in a cytotoxicity assay.
C44. The method according to Clause C1, wherein the composition induces a toxin A-specific neutralizing antibody concentration that is at least 2-fold higher in the human after receiving the second dose than a toxin A-specific neutralizing antibody concentration in the human prior to receiving the first dose, when measured under identical conditions in a cytotoxicity assay.
C45. The method according to Clause C1, wherein the composition induces a toxin A-specific neutralizing antibody concentration that is at least 4-fold higher in the human after receiving the second dose than a toxin A-specific neutralizing antibody concentration in the human prior to receiving the first dose, when measured under identical conditions in a cytotoxicity assay.
C46. The method according to Clause C1, wherein the composition induces a toxin A-specific neutralizing antibody concentration that is at least 8-fold higher in the human after receiving the second dose than a toxin A-specific neutralizing antibody concentration in the human prior to receiving the first dose, when measured under identical conditions in a cytotoxicity assay.
C47. The method according to Clause C1, wherein the composition induces a toxin A-specific neutralizing antibody concentration that is at least 16-fold higher in the human after receiving the second dose than a toxin A-specific neutralizing antibody concentration in the human prior to receiving the first dose, when measured under identical conditions in a cytotoxicity assay.
C48. The method according to Clause C1, wherein the composition induces a toxin A-specific neutralizing antibody concentration that is at least 32-fold higher in the human after receiving the second dose than a toxin A-specific neutralizing antibody concentration in the human prior to receiving the first dose, when measured under identical conditions in a cytotoxicity assay.
C49. The method according to Clause C1, wherein the composition induces a toxin B-specific neutralizing antibody concentration that is at least 2-fold higher in the human after receiving the second dose than a toxin B-specific neutralizing antibody concentration in the human prior to receiving the first dose, when measured under identical conditions in a cytotoxicity assay.
C50. The method according to Clause C1, wherein the composition induces a toxin B-specific neutralizing antibody concentration that is at least 4-fold higher in the human after receiving the second dose than a toxin B-specific neutralizing antibody concentration in the human prior to receiving the first dose, when measured under identical conditions in a cytotoxicity assay.
C51. The method according to Clause C1, wherein the composition induces a toxin B-specific neutralizing antibody concentration that is at least 8-fold higher in the human after receiving the second dose than a toxin B-specific neutralizing antibody concentration in the human prior to receiving the first dose, when measured under identical conditions in a cytotoxicity assay.
C52. The method according to Clause C1, wherein the composition induces a toxin B-specific neutralizing antibody concentration that is at least 16-fold higher in the human after receiving the second dose than a toxin B-specific neutralizing antibody concentration in the human prior to receiving the first dose, when measured under identical conditions in a cytotoxicity assay.
C53. The method according to Clause C1, wherein the composition induces a toxin B-specific neutralizing antibody concentration that is at least 32-fold higher in the human after receiving the second dose than a toxin B-specific neutralizing antibody concentration in the human prior to receiving the first dose, when measured under identical conditions in a cytotoxicity assay.
C54. The method according to Clause C1, wherein the composition induces a toxin A-specific neutralizing antibody concentration that is at least 2-fold higher in the human after receiving the third dose than a toxin A-specific neutralizing antibody concentration in the human prior to receiving the first dose, when measured under identical conditions in a cytotoxicity assay.
C55. The method according to Clause C1, wherein the composition induces a toxin A-specific neutralizing antibody concentration that is at least 4-fold higher in the human after receiving the third dose than a toxin A-specific neutralizing antibody concentration in the human prior to receiving the first dose, when measured under identical conditions in a cytotoxicity assay.
C56. The method according to Clause C1, wherein the composition induces a toxin A-specific neutralizing antibody concentration that is at least 8-fold higher in the human after receiving the third dose than a toxin A-specific neutralizing antibody concentration in the human prior to receiving the first dose, when measured under identical conditions in a cytotoxicity assay.
C57. The method according to Clause C1, wherein the composition induces a toxin A-specific neutralizing antibody concentration that is at least 16-fold higher in the human after receiving the third dose than a toxin A-specific neutralizing antibody concentration in the human prior to receiving the first dose, when measured under identical conditions in a cytotoxicity assay.
C58. The method according to Clause C1, wherein the composition induces a toxin A-specific neutralizing antibody concentration that is at least 32-fold higher in the human after receiving the third dose than a toxin A-specific neutralizing antibody concentration in the human prior to receiving the first dose, when measured under identical conditions in a cytotoxicity assay.
C59. The method according to Clause C1, wherein the composition induces a toxin B-specific neutralizing antibody concentration that is at least 2-fold higher in the human after receiving the third dose than a toxin B-specific neutralizing antibody concentration in the human prior to receiving the first dose, when measured under identical conditions in a cytotoxicity assay.
C60. The method according to Clause C1, wherein the composition induces a toxin B-specific neutralizing antibody concentration that is at least 4-fold higher in the human after receiving the third dose than a toxin B-specific neutralizing antibody concentration in the human prior to receiving the first dose, when measured under identical conditions in a cytotoxicity assay.
C61. The method according to Clause C1, wherein the composition induces a toxin B-specific neutralizing antibody concentration that is at least 8-fold higher in the human after receiving the third dose than a toxin B-specific neutralizing antibody concentration in the human prior to receiving the first dose, when measured under identical conditions in a cytotoxicity assay.
C62. The method according to Clause C1, wherein the composition induces a toxin B-specific neutralizing antibody concentration that is at least 16-fold higher in the human after receiving the third dose than a toxin B-specific neutralizing antibody concentration in the human prior to receiving the first dose, when measured under identical conditions in a cytotoxicity assay.
C63. The method according to Clause C1, wherein the composition induces a toxin B-specific neutralizing antibody concentration that is at least 32-fold higher in the human after receiving the third dose than a toxin B-specific neutralizing antibody concentration in the human prior to receiving the first dose, when measured under identical conditions in a cytotoxicity assay.
C64. The method according to Clause C1, wherein the composition induces a toxin A-specific neutralizing antibody concentration that is at least 2-fold higher in the human after receiving the first dose when measured about 7, 30, 60, 90, 120, 365, or 540 days after the first dose than a toxin A-specific neutralizing antibody concentration in the human prior to receiving the first dose, when measured under identical conditions in a cytotoxicity assay.
C65. The method according to Clause C1, wherein the composition induces a toxin A-specific neutralizing antibody concentration that is at least 2-fold higher in the human after receiving the second dose when measured about 7, 30, 60, 90, 120, 365, or 540 days after the second dose than a toxin A-specific neutralizing antibody concentration in the human prior to receiving the first dose, when measured under identical conditions in a cytotoxicity assay.
C66. The method according to Clause C1, wherein the composition induces a toxin A-specific neutralizing antibody concentration that is at least 2-fold higher in the human after receiving the third dose when measured about 7, 30, 60, 90, 120, 365, or 540 days after the third dose than a toxin A-specific neutralizing antibody concentration in the human prior to receiving the first dose, when measured under identical conditions in a cytotoxicity assay.
C67. The method according to Clause C1, wherein the composition induces a toxin B-specific neutralizing antibody concentration that is at least 2-fold higher in the human after receiving the first dose when measured about 7, 30, 60, 90, 120, 365, or 540 days after the first dose than a toxin B-specific neutralizing antibody concentration in the human prior to receiving the first dose, when measured under identical conditions in a cytotoxicity assay.
C68. The method according to Clause C1, wherein the composition induces a toxin B-specific neutralizing antibody concentration that is at least 2-fold higher in the human after receiving the second dose when measured about 7, 30, 60, 90, 120, 365, or 540 days after the second dose than a toxin B-specific neutralizing antibody concentration in the human prior to receiving the first dose, when measured under identical conditions in a cytotoxicity assay.
C69. The method according to Clause C1, wherein the composition induces a toxin B-specific neutralizing antibody concentration that is at least 2-fold higher in the human after receiving the third dose when measured on about any one of 7, 30, 60, 90, 120, 365, or 540 days after the third dose than a toxin B-specific neutralizing antibody concentration in the human prior to receiving the first dose, when measured under identical conditions in a cytotoxicity assay.
C70. The method according to Clause C1, wherein the composition induces a toxin B-specific neutralizing antibody concentration in the human after receiving the third dose when measured on about any one of 7, 30, 60, 90, 120, 365, or 540 days after the third dose than a toxin B-specific neutralizing antibody concentration in the human prior to receiving the first dose, when measured under identical conditions in a cytotoxicity assay.
C71. The method according to Clause C1, wherein the human is seronegative for toxin B.
C72. The method according to Clause C1, wherein the human is seronegative for toxin A.
C73. The method according to Clause C1, wherein the human is seronegative for toxin A and toxin B.
C74. The method according to Clause C1, wherein the human is seropositive for toxin B.
C75. The method according to Clause C1, wherein the human is seropositive for toxin A.
C76. The method according to Clause C1, wherein the human is seropositive for toxin A and toxin B.
C77. The method according to Clause C1, wherein the toxoid comprises SEQ ID NO: 4, wherein the methionine is absent.
C78. The method according to Clause C1, wherein the toxoid comprises SEQ ID NO: 6, wherein the methionine is absent.
C79. The method according to Clause C1, wherein the toxoid comprises any one of SEQ ID NOs: 1-8, 15, 17, 19, 21, 23, 25, 28-35, 82-761, wherein the methionine is absent.
C80. The method according to Clause C1, wherein the toxoid comprises a formaldehyde-contacted C. difficile toxin A.
C81. The method according to Clause C1, wherein the toxoid comprises a formaldehyde-contacted C. difficile toxin B.
C82. The method according to Clause C1, wherein the toxoid is not contacted with formaldehyde.
C83. The method according to Clause C1, wherein the infection is from a C. difficile Ribotype 002.
C84. The method according to Clause C1, wherein the infection is from a C. difficile Ribotype 003.
C85. The method according to Clause C1, wherein the infection is from a C. difficile Ribotype 004.
C86. The method according to Clause C1, wherein the infection is from a C. difficile Ribotype 012.
C87. The method according to Clause C1, wherein the infection is from a C. difficile Ribotype 015.
C88. The method according to Clause C1, wherein the infection is from a C. difficile Ribotype 017.
C89. The method according to Clause C1, wherein the infection is from a C. difficile Ribotype 020.
C90. The method according to Clause C1, wherein the infection is from a C. difficile Ribotype 023.
C91. The method according to Clause C1, wherein the infection is from a C. difficile Ribotype 027.
C92. The method according to Clause C1, wherein the infection is from a C. difficile Ribotype 029.
C93. The method according to Clause C1, wherein the infection is from a C. difficile Ribotype 046.
C94. The method according to Clause C1, wherein the infection is from a C. difficile Ribotype 053.
C95. The method according to Clause C1, wherein the infection is from a C. difficile Ribotype 059.
C96. The method according to Clause C1, wherein the infection is from a C. difficile Ribotype 070.
C97. The method according to Clause C1, wherein the infection is from a C. difficile Ribotype 075.
C98. The method according to Clause C1, wherein the infection is from a C. difficile Ribotype 078.
C99. The method according to Clause C1, wherein the infection is from a C. difficile Ribotype 081.
C100. The method according to Clause C1, wherein the infection is from a C. difficile Ribotype 087.
C101. The method according to Clause C1, wherein the infection is from a C. difficile Ribotype 106.
C102. The method according to Clause C1, wherein the infection is from a C. difficile Ribotype 117.
C103. The method according to Clause C1, wherein the infection is from a C. difficile Ribotype 126.
C104. The method according to Clause C1, wherein the infection is from a C. difficile Ribotype 131.
C105. The method according to Clause C1, wherein the infection is from a C. difficile Ribotype 154.
C106. The method according to Clause C1, wherein the infection is from a C. difficile Toxinotype 0.
C107. The method according to Clause C1, wherein the infection is from a C. difficile Toxinotype I.
C108. The method according to Clause C1, wherein the infection is from a C. difficile Toxinotype VIII.
C109. The method according to Clause C1, wherein the infection is from a C. difficile Toxinotype IV.
C110. The method according to Clause C1, wherein the infection is from a C. difficile Toxinotype III.
C111. The method according to Clause C1, wherein the infection is from a C. difficile Toxinotype XIII.
C112. The method according to Clause C1, wherein the infection is from a C. difficile Toxinotype V.

1. A method for eliciting an immune response in a human against a Clostridium difficile toxin selected from the group consisting of toxin A and toxin B, the method comprising administering to the human an effective dose of a composition, which comprises a C. difficile toxoid, wherein the composition is administered at least two times, wherein the second administration is about 30 days after the first administration, and wherein the immune response against the C. difficile toxin is sustained for at least about 60 days after the first dose; wherein the toxoid comprises a polypeptide having the amino acid sequence set forth in any one of SEQ ID NOs: 1-8, 15, 17, 19, 21, 23, 25, 28-35, and 82-761.

2. The method according to claim 1, wherein the composition is administered at least three times.

3. The method according to claim 1, wherein the third administration is about 180 days after the first administration.

4. The method according to claim 1, wherein the immune response elicited comprises an anti-toxin A neutralizing monoclonal antibody.

5. The method according to claim 1, wherein the immune response elicited comprises an anti-toxin B neutralizing monoclonal antibody.

6. The method according to claim 1, wherein the immune response elicited comprises an anti-toxin A neutralizing monoclonal antibody and an anti-toxin B neutralizing monoclonal antibody, wherein the concentration of neutralizing monoclonal antibody is at least 10 μg/mL.

7. The method according to claim 1, wherein the composition comprises a C. difficile toxoid A and/or a C. difficile toxoid B, each having a purity of at least 90% or greater.

8. The method according to claim 1, wherein the composition comprises a C. difficile toxoid A and a C. difficile toxoid B, in a ratio of about 3:1 to about 1:1.

9. The method according to claim 1 wherein the composition comprises a C. difficile toxoid A and a C. difficile toxoid B, in a ratio of 1:1.

10. The method according to claim 1, wherein the composition comprises an adjuvant.

11. The method according to claim 1, wherein the composition comprises an aluminum adjuvant.

12. The method according to claim 1, wherein the immune response against C. difficile toxin A and/or toxin B is sustained for at least about 180 days.

13. The method according to claim 1, wherein the immune response against C. difficile toxin A and/or toxin B is sustained for at least about 365 days.

14. The method according to claim 1, wherein the immune response against C. difficile toxin A and/or toxin B is sustained for at least about 540 days.

15. The method according to claim 1, wherein the composition comprises a C. difficile toxoid A and a C. difficile toxoid B, wherein the C. difficile toxoid A and C. difficile toxoid B are lyophilized.

16. The method according to claim 1, wherein the composition induces a toxin A-specific neutralizing antibody concentration that is at least 2-fold higher in the human after receiving the first dose than a toxin A-specific neutralizing antibody concentration in the human prior to receiving the first dose, when measured under identical conditions in a cytotoxicity assay.

17. The method according to claim 1, wherein the composition induces a toxin A-specific neutralizing antibody concentration that is at least 32-fold higher in the human after receiving the first dose than a toxin A-specific neutralizing antibody concentration in the human prior to receiving the first dose, when measured under identical conditions in a cytotoxicity assay.

18. The method according to claim 1, wherein the composition induces a toxin B-specific neutralizing antibody concentration that is at least 2-fold higher in the human after receiving the first dose than a toxin B-specific neutralizing antibody concentration in the human prior to receiving the first dose, when measured under identical conditions in a cytotoxicity assay.

19. The method according to claim 1, wherein the composition induces a toxin B-specific neutralizing antibody concentration that is at least 32-fold higher in the human after receiving the first dose than a toxin B-specific neutralizing antibody concentration in the human prior to receiving the first dose, when measured under identical conditions in a cytotoxicity assay.

20. The method according to claim 1, wherein the composition induces a toxin A-specific neutralizing antibody concentration that is at least 2-fold higher in the human after receiving the second dose than a toxin A-specific neutralizing antibody concentration in the human prior to receiving the first dose, when measured under identical conditions in a cytotoxicity assay.

21. The method according to claim 1, wherein the composition induces a toxin A-specific neutralizing antibody concentration that is at least 32-fold higher in the human after receiving the second dose than a toxin A-specific neutralizing antibody concentration in the human prior to receiving the first dose, when measured under identical conditions in a cytotoxicity assay.

22. The method according to claim 1, wherein the composition induces a toxin B-specific neutralizing antibody concentration that is at least 2-fold higher in the human after receiving the second dose than a toxin B-specific neutralizing antibody concentration in the human prior to receiving the first dose, when measured under identical conditions in a cytotoxicity assay.

23. The method according to claim 1, wherein the composition induces a toxin B-specific neutralizing antibody concentration that is at least 32-fold higher in the human after receiving the second dose than a toxin B-specific neutralizing antibody concentration in the human prior to receiving the first dose, when measured under identical conditions in a cytotoxicity assay.

24. The method according to claim 1, wherein the human is seronegative for toxin B.

25. The method according to claim 1, wherein the human is seronegative for toxin A.

26. The method according to claim 1, wherein the human is seronegative for toxin A and toxin B.

27. The method according to claim 1, wherein the human is seropositive for toxin B.

28. The method according to claim 1, wherein the human is seropositive for toxin A.

29. The method according to claim 1, wherein the human is seropositive for toxin A and toxin B.

30. The method according to claim 1, wherein the composition further comprises QS-21.

31. The method according to claim 1, wherein the human has an antibody titer against a polypeptide comprising SEQ ID NO: 1 of at least 219 neutralization units/mL.

32. The method according to claim 1, wherein the human has an antibody titer against a polypeptide comprising SEQ ID NO: 2 of at least 2586 neutralization units/mL.

33. A method for eliciting an immune response in a human against a Clostridium difficile selected from the group consisting of Ribotype 002, Ribotype 003, Ribotype 004, Ribotype 012, Ribotype 015, Ribotype 017, Ribotype 020, Ribotype 023, Ribotype 027, Ribotype 029, Ribotype 046, Ribotype 053, Ribotype 059, Ribotype 070, Ribotype 075, Ribotype 078, Ribotype 081, Ribotype 087, Ribotype 106, Ribotype 117, Ribotype 126, Ribotype 131, Ribotype 154, Toxinotype 0, Toxinotype I, Toxinotype VIII, Toxinotype IV, Toxinotype III, Toxinotype XIII, Toxinotype V; the method comprising administering to the human an effective dose of a composition, which comprises a C. difficile toxoid; wherein the toxoid comprises a polypeptide having the amino acid sequence set forth in any one of SEQ ID NOs: 1-8, 15, 17, 19, 21, 23, 25, 28-35, and 82-761.
